(19) United States
(12) Patent Application Publication
(10) Pub. No.: US 2024/0024380 A1
(43) Pub. Date: Jan. 25, 2024

(54) BACTERIA-ENGINEERED TO ELICIT ANTIGEN-SPECIFIC T CELLS

(71) Applicants: CHAN ZUCKERBERG BIOHUB, INC., San Franicisco, CA (US); THE BOARD OF TRUSTEES OF THE LELAND STANFORD JUNIOR UNIVERSITY, Stanford, CA (US)

(72) Inventors: Michael A. Fischbach, Stanford, CA (US); Kazuki Nagashima, Palo Alto, CA (US); Yiyin E. Chen, Stanford, CA (US); Djenet Bousbaine, Mountain View, CA (US)

(21) Appl. No.: 18/269,237

(22) PCT Filed: Dec. 22, 2021
(86) PCT No.: PCT/US2021/065011

§ 371 Date: Jun. 22, 2023

Related U.S. Application Data
(60) Provisional application No. 63/150,013, filed on Feb. 16, 2021.
(60) Provisional application No. 63/130,354, filed on Dec. 23, 2020.
(60) Provisional application No. 63/130,356, filed on Dec. 23, 2020.

(51) Int. Cl. A61K 35/74; A61K 45/06; C07K 16/28; A61P 35/04

(52) U.S. Cl. CPC A61K 35/74; A61K 45/06; C07K 16/2845; C07K 16/2833; A61P 35/04; C07K 2319/02; C07K 2319/10

(57) ABSTRACT

Provided are modified microorganisms, such as live recombinant commensal bacteria, that express a non-native antigen, or are surface-labeled with a non-native antigen, and methods of using the modified microorganisms to induce an antigen-specific immune response to the non-native antigen. The modified microorganism can be used to induce a regulatory T cell immune response to the heterologous antigen to treat an autoimmune disease in a subject in need thereof, or can be used to induce an effector T cell immune response to the heterologous antigen to treat an infectious disease or proliferative disease in a subject in need thereof.

### CROSS-REFERENCE TO RELATED APPLICATIONS

This application claims the benefit of and priority to U.S. Provisional Patent Application No. 63/130,354, filed Dec. 23, 2020; to U.S. Provisional Patent Application No. 63/130,356, filed Dec. 23, 2020; and to U.S. Provisional Patent Application No. 63/150,013, filed Feb. 16, 2021, the disclosures of which are hereby incorporated by reference in their entireties for all purposes.

### STATEMENT AS TO RIGHTS TO INVENTIONS MADE UNDER FEDERALLY SPONSORED RESEARCH AND DEVELOPMENT

This invention was made with Government support under Grant No: DK113598 awarded by the National Institutes of Health (NIH). The Government has certain rights in the invention.

### SEQUENCE LISTING

The instant application contains a Sequence Listing which has been submitted electronically in ASCII format and is hereby incorporated by reference in its entirety. Said ASCII copy, created on Dec. 22, 2021, is named FBI-005WO_SL_ST25.txt and is 56,726 bytes in size.

### FIELD OF THE INVENTION

The invention generally relates to modified bacteria and methods of using such bacteria to elicit antigen-specific adaptive immune responses for the treatment of a disease or condition in a subject.

### BACKGROUND OF THE INVENTION

Commensal microbiota reside primarily at barrier sites, such as the gastrointestinal tract, respiratory tract, urogenital tract and skin, where they functionally tune the innate and adaptive immune systems. Immune tolerance to these microbes must be established at each of these sites. In the gastrointestinal tract, a simple columnar epithelium is coated by a thick mucus layer that facilitates spatial segregation from luminal bacteria and also diminishes the immunogenicity of microbial antigens by delivering tolerogenic signals to resident dendritic cells. Innate lymphoid cells limit commensal-specific CD4+ T cell responses via an MHC-II-dependent mechanism and produce interleukin-22, which further promotes anatomical containment of microbes. Specialized gut-resident CD103+CD11b+ dendritic cells also play an important role in maintaining intestinal homeostasis by favoring induction of regulatory T (T_{reg}) cells over pro-inflammatory CD4+ subsets (see Scharschmidt T. C. et al., Immunity 2015, November 17; 43(5): 1011-1021). Interestingly, in other microbial niches such as the skin, certain commensal microbes (e.g., Staphylococcus epidermidis) have been demonstrated to selectively induce a CD8+ effector T cell response via interaction with dermal dendritic cells (see Naik S. et al., Nature 2015, 520:104-108).

T_{reg} cells play a major role in establishing and maintaining immune homeostasis in peripheral tissues, particularly at barrier sites where they stably reside. In the intestinal lamina propria, T_{reg} cells not only maintain self-tolerance but also play a crucial role in mediating tolerance to commensal organisms. A large percentage of gut-resident T_{reg} cells recognize commensal antigens, and thymically derived T_{reg} cells support tolerance to intestinal microbes. In addition, certain bacterial species expand T_{reg} cells in the lamina propria (Id.).

T_{regs} are a subset of T helper (T_{H}) cells, and are considered to be derived from the same lineage as naïve CD4 cells. T_{regs} are involved in maintaining tolerance to self-antigens, and preventing auto-immune disease. T_{regs} also suppress induction and proliferation of effector T cells (T_{eff}). T_{regs} produce inhibitory cytokines such as TGF-β, IL-35, and IL-10. T_{regs} express the transcription factor Foxp3. In humans, the majority of T_{reg} cells are MHC-II restricted CD4+ cells, but there is a minority population that are FoxP3+, MHC-I restricted, CD8+ cells. T_{regs} can also be divided into subsets: “natural” CD4+CD25+ FoxP3+T_{reg} cells (nT_{regs}) that develop in the thymus, and “inducible” regulatory cells (iT_{regs}) which arise in the periphery. iT_{regs} are also CD4+CD25+ FoxP3+, and develop from mature CD4+ T cells in the periphery (i.e., outside of the thymus). iT_{regs} can also express both RORγt and Foxp3 (see Sefik E., et al., “Individual intestinal symbionts induce a distinct population of RORgamma(+) regulatory T cells,” Science 2015; 349:993-997). Research has shown that TGF-β and retinoic acid produced by dendritic cells can stimulate naïve T cells to differentiate into T_{regs}, and that naïve T cells within the digestive tract differentiate into T_{regs} after antigen stimulation. iT_{regs} can also be induced in culture by adding TGF-β.

In contrast to T_{regs}, T effector (T_{eff}) cells generally stimulate a pro-inflammatory response upon antigen-specific T Cell receptor (TCR) activation via the expression or release of an array of membrane-bound and secreted proteins that are specialized to deal with different classes of pathogen. There are three classes of T_{eff} cell: CD8+ cytotoxic T cells, T_{H}1 cells, and T_{H}2 cells. CD8+ cytotoxic T cells recognize and kill target cells that display peptide fragments of intracellular pathogens (e.g., viruses) presented in the context of MHC-I molecules at the cell surface. CD8+ cytotoxic T cells store preformed cytotoxins in lytic granules which fuse with the membranes of infected target cells. CD8+ cytotoxic T cells additionally express Fas ligand, which induces apoptosis in Fas-expressing target cells. T_{H}1 and T_{H}2 cells both express CD4 and recognize peptide fragments degraded within intracellular vesicles and presented on the cell surface in the context of MHC-II molecules. T_{H}1 cells can activate a number of other immune cells, including macrophages and B cells, thereby promoting more efficient destruction and clearance of intracellular microorganisms. T_{H}2 cells stimulate the differentiation of B cells and promote the production of antibodies and other effector molecules of the humoral immune response.

### SUMMARY OF THE INVENTION

The present disclosure is directed to compositions and methods of use thereof for a recombinant bacterium expressing a non-native protein or peptide to promote an immune response against a specified antigen.

Provided herein is a composition comprising a live, recombinant commensal bacterium, wherein the bacterium is engineered to express a fusion protein comprising (a) a non-native protein or peptide and (b) a tat signal sequence peptide, a sec signal sequence peptide, or a sortase-derived signal sequence peptide, wherein the non-native protein or peptide is associated with a host disease or condition, wherein upon administration of the bacterium to the host resulting in colonization of a native host niche by the bacterium, the host mounts an adaptive immune response to the non-native protein or peptide, wherein the adaptive immune response is a T cell response. In some aspects, the colonization of the native host niche is persistent or transient. In some aspects, the native host niche is persistently colonized, and wherein colonization is for at least 60 days, at least 112 days, at least 178 days, at least 1 year, at least 2 years, or at least 5 years. In some aspects, the native host niche is persistently colonized, and wherein colonization is for at least 180 days. In some aspects, the persistent colonization provides a persistent antigen source, optionally wherein the antigen stimulates an antigen-specific T cell population and produces a persistent antigen-specific T cell population. In some aspects, the native host niche is transiently colonized, and wherein colonization is for 1 day to 60 days. In some aspects, the native host niche is transiently colonized, and wherein colonization is for 3.5 days to 60 days. In some aspects, the native host niche is transiently colonized, and wherein colonization is for 7 days to 28 days. In some aspects, colonization is determined by polymerase chain reaction or colony forming assay performed on a sample obtained from the host after 1 day, 3.5 days, 7 days, 14 days, 28 days, or 60 days after administration to the host.

In some aspects, administration results in interaction of the bacterium with a native immune system partner cell. In some aspects, the native immune system partner cell is an antigen-presenting cell. In some aspects, the antigen-presenting cell is selected from the group consisting of a dendritic cell, a macrophage, a B-cell, and an intestinal epithelial cell. In some aspects, the native host niche is selected from the group consisting of the gastrointestinal tract, respiratory tract, urogenital tract, and skin. In some aspects, the non-native protein or peptide is a host protein or peptide.

In some aspects, the bacterium is a Gram-negative bacterium. In some aspects, the Gram-negative bacterium is selected from the group consisting of Bacteroides thetaiotaomicron, Helicobacter hepaticus and Parabacteroides sp.

In some aspects, the bacterium is a Gram-positive bacterium. In some aspects, the Gram-positive bacterium is selected from the group consisting of Staphylococcus epidermidis, Faecalibacterium sp., Corynebacterium spp., Eubacterium limosum, Ruminococcaceae bacterium cv2, Clostridium sp., Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus AGR2154, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Ruminococcus bromii L2-63, Firmicutes bacterium ASF500, Firmicutes bacterium ASF500, Bifidobacterium animalis subsp. lactis ATCC 27673, and Bifidobacterium breve UCC2003. In some aspects, the Gram-positive bacterium is selected from the group consisting of Staphylococcus epidermidis, Faecalibacterium sp., Corynebacterium spp., and Clostridium sp. In some aspects, the bacterium is selected from the group consisting of Staphylococcus epidermidis and Corynebacterium spp. In some aspects, the bacterium is S. epidermidis NIHLM087.

In some aspects, the bacterium is selected from the group consisting of: Corynebacterium tuberculostearicum, Corynebacterium accolens, Corynebacterium amycolatum, Corynebacterium aurimucosum, Corynebacterium propinquum, Corynebacterium pseudodiphtheriticum, Corynebacterium granulosum, Cutibacterium acnes, Cutibacterium avidum, Dolosigranulum pigrum, Finegoldia magna, Moraxella catarrhalis, Moraxella nonliquefaciens, Haemophilus influenzae, Haemophilus aegyptius, Rothia mucilaginosa, Streptococcus pyogenes, Streptococcus agalactiae, Streptococcus gordonii, Neisseria lactamica, Neisseria cinerea, Neisseria mucosa, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium breve, Bifidobacterium longum, Veillonella parvula, Gardnerella vaginalis, Atopobium vaginae, Prevotella bivia, Mobiluncus mulieris, Mageeibacillus indolicus, Prevotella buccalis, Enterococcus faecium, Lactococcus lactis, Ruminococcus gnavus, and Eubacterium limosum. In some aspects, the commensal bacterium is selected from the group consisting of: a bacterium having ATCC accession number 35692, 49725, 49726, 49368, 700975, 700540, 51488, 10700, 25564, 51277, 11827, 25577, 49753, 51524, 29328, 25238, 25240, 19976, 51907, 11116, 25296, 19615, 12344, BAA-611, 13813, 10558, 23970, 14685, 19696, 33820, 25258, 19992, 55195, 4356, 33200, 7469, 393, 7995D-5, 23272, 11741, 15700, 15697, 10790, 17745, 14018, BAA-55, 29303, 35243, BAA-2120, 35310, 19434, 19435, 29149, and 8486. In some aspects, the commensal bacterium is selected from the group consisting of: Lactobacillus casei, Lactococcus lactis, Streptococcus gordonii, Lactobacillus crispatus, Lactobacillus iners, Cutibacterium acnes, Streptococcus agalactiae, Ruminococcus gnavus, Neisseria lactamica, Bifidobacterium breve, and Bifidobacterium longum. In some aspects, the commensal bacterium is selected from the group consisting of: a bacterium having ATCC accession number 393, 19435, 35105, 33820, 55195, 6919, 13813, 23970, 15700, and 15707, and a bacterium having an accession number JCM6515.

In some aspects, wherein the administration is via a route selected from the group consisting of topical, enteral, and inhalation. In some aspects, the route is topical. In some aspects, the route is enteral.

In some aspects, the protein or peptide is associated with an infection. In some aspects, the infection is selected from the group consisting of a viral infection, a parasitic infection, a bacterial infection, or a fungal infection. In some aspects, the infection occurs at or is otherwise associated with a mucosal boundary of the host. In some aspects, the non-native protein or peptide is derived from a virus, a parasite, a bacterium, or a fungus associated with the infection. In some aspects, the non-native protein or peptide is derived from influenza, HSV, HIV, or SARS-Cov-2. In some aspects, the non-native protein or peptide is selected from the group consisting of: NP366-374, NP306-322, NA177-193, M2 ectodomain, HA2 stem-HA2 12-63, HA2 stem-HA2 76-130, gB glycoprotein, gd glycoprotein, gB glycoprotein 498-505, SARS-Cov2 Spike protein, HIV-gp120, HIV-gp41, HIV V1V2 apex, HIV V3 loop, HIV CD4 binding site, gp120/gp41 interface, gp120 silent face, and HIV membrane-proximal external region (MPER).

In some aspects, the protein or peptide is associated with an autoimmune disorder.

In some aspects, the protein or peptide is associated with a proliferative disorder. In some aspects, the proliferative disorder is cancer. In some aspects, the cancer is selected from melanoma, basal cell carcinoma, squamous cell carcinoma, testicular cancer, sarcoma, and prostate cancer. In some aspects, the cancer is melanoma. In some aspects, the non-native protein or peptide is derived from a melanocyte-specific antigen selected from the group consisting of PMEL, TRP2 and MART-1.

In some aspects, the non-native protein or peptide comprises a neoantigen, wherein the neoantigen comprises at least one mutation that makes the non-native protein or peptide distinct from a protein or peptide encoded by a wild-type gene of the host. In some aspects, wherein the neoantigen is selected from the group consisting of: Ints11, Kif18 bp, T3 sarcoma neoantigens, and a neoantigen expressed by the TRAMPC2 prostate cancer cell line.

In some aspects, the fusion protein further comprises a signal sequence peptide. In some aspects, the signal sequence peptide directs tethering of the fusion protein to a cell wall of the bacterium following expression. In some aspects, the signal sequence peptide that directs secretion comprises a tat signal sequence peptide. In some aspects, the tat signal sequence peptide comprises an S. aureus derived signal sequence peptide. In some aspects, the signal sequence peptide that directs secretion comprises a sec signal sequence peptide. In some aspects, the sec signal sequence peptide comprises an S. epidermidis derived signal sequence peptide. In some aspects, the S. epidermidis derived signal sequence peptide is derived from predicted sec-secreted S. epidermidis protein (gene locus HMPREF9993_06668).

In some aspects, the fusion protein further comprises an antigen-presenting cell (APC) targeting moiety, optionally wherein the APC targeting moiety comprises a CD11b or a MHC II targeting moiety. In some aspects, the APC targeting moiety comprises a nanobody (VHH) antibody binding domain, optionally wherein the VHH antibody binding domain comprises the sequence

(SEQ ID NO: 33)
QVQLQESGGGLVQAGDSLRLSCAASGRTFSRGVMGWFRRAPGKEREFVA
IFSGSSWSGRSTYYSDSVKGRFTISRDNAKNTVYLQMNGLKPEDTAVYY
CAAGYPEAYSAYGRESTYDYWGQGTQVTVSSGG
or
(SEQ ID NO: 34)
QVQLQESGGGLVQAGGSHNLSCTASGITFSSLAMGWFRQTPGKEREFVA
NIMRSGSSVFYADSVRGRFTISRDNAKNTAHLQMNSLKPEDTAVYFCAA
TRGAWPAEYWGQGTQVTVSSGG.

In some aspects, the bacterium is engineered to express a fusion protein comprising the protein or peptide and a native bacterial protein or portion thereof. In some aspects, the protein or peptide is fused to the N-terminus or the C-terminus of the native bacterial protein or portion thereof. In some aspects, the bacterium is formulated for administration in combination with a high-complexity defined microbial community.

In some aspects, the host is a mammal. In some aspects, the mammal is a human.

Also provided herein is a composition comprising a live, recombinant commensal bacterium, wherein the bacterium is engineered to express a fusion protein comprising (a) a non-native protein or peptide and (b) an antigen-presenting cell (APC) targeting moiety. In some aspects, the non-native protein or peptide is associated with a host disease or condition, wherein upon administration of the bacterium to the host resulting in colonization of a native host niche by the bacterium, the host mounts an adaptive immune response to the non-native protein or peptide. In some aspects, wherein the adaptive immune response is a T cell response or a B cell response. In some aspects, the colonization of the native host niche is persistent or transient. In some aspects, the native host niche is persistently colonized, and wherein colonization is for at least 60 days, at least 112 days, at least 178 days, at least 1 year, at least 2 years, or at least 5 years. In some aspects, the native host niche is persistently colonized, and wherein colonization is for at least 180 days. In some aspects, the persistent colonization provides a persistent antigen source, optionally wherein the antigen stimulates an antigen-specific T cell population and produces a persistent antigen-specific T cell population. In some aspects, the native host niche is transiently colonized, and wherein colonization is for 1 day to 60 days. In some aspects, the native host niche is transiently colonized, and wherein colonization is for 3.5 days to 60 days. In some aspects, the native host niche is transiently colonized, and wherein colonization is for 7 days to 28 days. In some aspects, colonization is determined by polymerase chain reaction or colony forming assay performed on a sample obtained from the host after 1 day, 3.5 days, 7 days, 14 days, 28 days, or 60 days after administration to the host.

In some aspects, administration results in interaction of the bacterium with a native immune system partner cell. In some aspects, the native immune system partner cell is an antigen-presenting cell. In some aspects, the antigen-presenting cell is selected from the group consisting of a dendritic cell, a macrophage, a B-cell, and an intestinal epithelial cell. In some aspects, the native host niche is selected from the group consisting of the gastrointestinal tract, respiratory tract, urogenital tract, and skin. In some aspects, the non-native protein or peptide is a host protein or peptide.

In some aspects, the bacterium is a Gram-negative bacterium. In some aspects, the Gram-negative bacterium is selected from the group consisting of Bacteroides thetaiotaomicron, Helicobacter hepaticus and Parabacteroides sp.

In some aspects, the bacterium is a Gram-positive bacterium. In some aspects, the Gram-positive bacterium is selected from the group consisting of Staphylococcus epidermidis, Faecalibacterium sp., Corynebacterium spp., Eubacterium limosum, Ruminococcaceae bacterium cv2, Clostridium sp., Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus AGR2154, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Ruminococcus bromii L2-63, Firmicutes bacterium ASF500, Firmicutes bacterium ASF500, Bifidobacterium animalis subsp. lactis ATCC 27673, and Bifidobacterium breve UCC2003. In some aspects, the Gram-positive bacterium is selected from the group consisting of Staphylococcus epidermidis, Faecalibacterium sp., Corynebacterium spp., and Clostridium sp. In some aspects, the bacterium is selected from the group consisting of Staphylococcus epidermidis and Corynebacterium spp. In some aspects, the bacterium is S. epidermidis NIHLM087.

In some aspects, the bacterium is selected from the group consisting of: Corynebacterium tuberculostearicum, Corynebacterium accolens, Corynebacterium amycolatum, Corynebacterium aurimucosum, Corynebacterium propinquum, Corynebacterium pseudodiphtheriticum, Corynebacterium granulosum, Cutibacterium acnes, Cutibacterium avidum, Dolosigranulum pigrum, Finegoldia magna, Moraxella catarrhalis, Moraxella nonliquefaciens, Haemophilus influenzae, Haemophilus aegyptius, Rothia mucilaginosa, Streptococcus pyogenes, Streptococcus agalactiae, Streptococcus gordonii, Neisseria lactamica, Neisseria cinerea, Neisseria mucosa, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium breve, Bifidobacterium longum, Veillonella parvula, Gardnerella vaginalis, Atopobium vaginae, Prevotella bivia, Mobiluncus mulieris, Mageeibacillus indolicus, Prevotella buccalis, Enterococcus faecium, Lactococcus lactis, Ruminococcus gnavus, and Eubacterium limosum. In some aspects, the commensal bacterium is selected from the group consisting of: a bacterium having ATCC accession number 35692, 49725, 49726, 49368, 700975, 700540, 51488, 10700, 25564, 51277, 11827, 25577, 49753, 51524, 29328, 25238, 25240, 19976, 51907, 11116, 25296, 19615, 12344, BAA-611, 13813, 10558, 23970, 14685, 19696, 33820, 25258, 19992, 55195, 4356, 33200, 7469, 393, 7995D-5, 23272, 11741, 15700, 15697, 10790, 17745, 14018, BAA-55, 29303, 35243, BAA-2120, 35310, 19434, 19435, 29149, and 8486. In some aspects, the commensal bacterium is selected from the group consisting of: Lactobacillus casei, Lactococcus lactis, Streptococcus gordonii, Lactobacillus crispatus, Lactobacillus iners, Cutibacterium acnes, Streptococcus agalactiae, Ruminococcus gnavus, Neisseria lactamica, Bifidobacterium breve, and Bifidobacterium longum. In some aspects, the commensal bacterium is selected from the group consisting of: a bacterium having ATCC accession number 393, 19435, 35105, 33820, 55195, 6919, 13813, 23970, 15700, and 15707, and a bacterium having an accession number JCM6515.

In some aspects, wherein the administration is via a route selected from the group consisting of topical, enteral, and inhalation. In some aspects, the route is topical. In some aspects, the route is enteral.

In some aspects, the protein or peptide is associated with an infection. In some aspects, the infection is selected from the group consisting of a viral infection, a parasitic infection, a bacterial infection, or a fungal infection. In some aspects, the infection occurs at or is otherwise associated with a mucosal boundary of the host. In some aspects, the non-native protein or peptide is derived from a virus, a parasite, a bacterium, or a fungus associated with the infection. In some aspects, the non-native protein or peptide is derived from influenza, HSV, HIV, or SARS-Cov-2. In some aspects, the non-native protein or peptide is selected from the group consisting of: NP366-374, NP306-322, NA177-193, M2 ectodomain, HA2 stem-HA2 12-63, HA2 stem-HA2 76-130, gB glycoprotein, gd glycoprotein, gB glycoprotein 498-505, SARS-Cov2 Spike protein, HIV-gp120, HIV-gp41, HIV V1V2 apex, HIV V3 loop, HIV CD4 binding site, gp120/gp41 interface, gp120 silent face, and HIV membrane-proximal external region (MPER).

In some aspects, the protein or peptide is associated with an autoimmune disorder.

In some aspects, the protein or peptide is associated with a proliferative disorder. In some aspects, the proliferative disorder is cancer. In some aspects, the cancer is selected from melanoma, basal cell carcinoma, squamous cell carcinoma, testicular cancer, sarcoma, and prostate cancer. In some aspects, the cancer is melanoma. In some aspects, the non-native protein or peptide is derived from a melanocyte-specific antigen selected from the group consisting of PMEL, TRP2 and MART-1.

In some aspects, the non-native protein or peptide comprises a neoantigen, wherein the neoantigen comprises at least one mutation that makes the non-native protein or peptide distinct from a protein or peptide encoded by a wild-type gene of the host. In some aspects, wherein the neoantigen is selected from the group consisting of: Ints11, Kif18 bp, T3 sarcoma neoantigens, and a neoantigen expressed by the TRAMPC2 prostate cancer cell line.

In some aspects, the fusion protein further comprises a signal sequence peptide. In some aspects, the signal sequence peptide directs tethering of the fusion protein to a cell wall of the bacterium following expression. In some aspects, the signal sequence peptide that directs secretion comprises a tat signal sequence peptide. In some aspects, the tat signal sequence peptide comprises an S. aureus derived signal sequence peptide. In some aspects, the signal sequence peptide that directs secretion comprises a sec signal sequence peptide. In some aspects, the sec signal sequence peptide comprises an S. epidermidis derived signal sequence peptide. In some aspects, the S. epidermidis derived signal sequence peptide is derived from predicted sec-secreted S. epidermidis protein (gene locus HMPREF9993_06668).

In some aspects, the bacterium is engineered to express a fusion protein comprising the protein or peptide and a native bacterial protein or portion thereof. In some aspects, the protein or peptide is fused to the N-terminus or the C-terminus of the native bacterial protein or portion thereof. In some aspects, the bacterium is formulated for administration in combination with a high-complexity defined microbial community.

In some aspects, the host is a mammal. In some aspects, the mammal is a human.

Also provided herein is a composition comprising a live, recombinant commensal bacterium, wherein the bacterium is engineered to express a fusion protein comprising a non-native protein or peptide, wherein the non-native protein or peptide is associated with a host disease or condition, wherein upon administration of the bacterium to the host resulting in colonization of a native host niche by the bacterium, the host mounts an adaptive immune response to the non-native protein or peptide, and wherein the commensal bacterium is selected from the group consisting of: Corynebacterium tuberculostearicum, Corynebacterium accolens, Corynebacterium amycolatum, Corynebacterium aurimucosum, Corynebacterium propinquum, Corynebacterium pseudodiphtheriticum, Corynebacterium granulosum, Cutibacterium acnes, Cutibacterium avidum, Dolosigranulum pigrum, Finegoldia magna, Moraxella catarrhalis, Moraxella nonliquefaciens, Haemophilus influenzae, Haemophilus aegyptius, Rothia mucilaginosa, Streptococcus pyogenes, Streptococcus agalactiae, Streptococcus gordonii, Neisseria lactamica, Neisseria cinerea, Neisseria mucosa, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium breve ATCC 15700, Bifidobacterium longum, Veillonella parvula, Gardnerella vaginalis, Atopobium vaginae, Prevotella bivia, Mobiluncus mulieris, Mageeibacillus indolicus, Prevotella buccalis, Enterococcus faecium, Lactococcus lactis, Ruminococcus gnavus JCM6515, and Eubacterium limosum ATCC 8486. In some aspects, the commensal bacterium is selected from the group consisting of: a bacterium having ATCC accession number 35692, 49725, 49726, 49368, 700975, 700540, 51488, 10700, 25564, 51277, 11827, 25577, 49753, 51524, 29328, 25238, 25240, 19976, 51907, 11116, 25296, 19615, 12344, BAA-611, 13813, 10558, 23970, 14685, 19696, 33820, 25258, 19992, 55195, 4356, 33200, 7469, 393, 7995D-5, 23272, 11741, 15700, 15697, 10790, 17745, 14018, BAA-55, 29303, 35243, BAA-2120, 35310, 19434, 19435, 29149, and 8486. In some aspects, the commensal bacterium is selected from the group consisting of: Lactobacillus casei, Lactococcus lactis, Streptococcus gordonii, Lactobacillus crispatus, Lactobacillus iners, Cutibacterium acnes, Streptococcus agalactiae, Ruminococcus gnavus JCM6515, Neisseria lactamica, Bifidobacterium breve ATCC 15700, and Bifidobacterium longum. In some aspects, the commensal bacterium is selected from the group consisting of: a bacterium having ATCC accession number 393, 19435, 35105, 33820, 55195, 6919, 13813, 23970, 15700, and 15707, and a bacterium having an accession number JCM6515. In some aspects, the adaptive immune response is a T cell response or a B cell response. In some aspects, the colonization of the native host niche is persistent or transient. In some aspects, the native host niche is persistently colonized, and wherein colonization is for at least 60 days, at least 112 days, at least 178 days, at least 1 year, at least 2 years, or at least 5 years. In some aspects, the native host niche is persistently colonized, and wherein colonization is for at least 180 days. In some aspects, the persistent colonization provides a persistent antigen source, optionally wherein the antigen stimulates an antigen-specific T cell population and produces a persistent antigen-specific T cell population. In some aspects, the native host niche is transiently colonized, and wherein colonization is for 1 day to 60 days. In some aspects, the native host niche is transiently colonized, and wherein colonization is for 3.5 days to 60 days. In some aspects, the native host niche is transiently colonized, and wherein colonization is for 7 days to 28 days. In some aspects, colonization is determined by polymerase chain reaction or colony forming assay performed on a sample obtained from the host after 1 day, 3.5 days, 7 days, 14 days, 28 days, or 60 days after administration to the host.

In some aspects, administration results in interaction of the bacterium with a native immune system partner cell. In some aspects, the native immune system partner cell is an antigen-presenting cell. In some aspects, the antigen-presenting cell is selected from the group consisting of a dendritic cell, a macrophage, a B-cell, and an intestinal epithelial cell. In some aspects, the native host niche is selected from the group consisting of the gastrointestinal tract, respiratory tract, urogenital tract, and skin. In some aspects, the non-native protein or peptide is a host protein or peptide.

In some aspects, the bacterium is a Gram-negative bacterium. In some aspects, the Gram-negative bacterium is selected from the group consisting of Bacteroides thetaiotaomicron, Helicobacter hepaticus and Parabacteroides sp.

In some aspects, the bacterium is a Gram-positive bacterium. In some aspects, the Gram-positive bacterium is selected from the group consisting of Staphylococcus epidermidis, Faecalibacterium sp., Corynebacterium spp., Eubacterium limosum, Ruminococcaceae bacterium cv2, Clostridium sp., Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus AGR2154, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Ruminococcus bromii L2-63, Firmicutes bacterium ASF500, Firmicutes bacterium ASF500, Bifidobacterium animalis subsp. lactis ATCC 27673, and Bifidobacterium breve UCC2003. In some aspects, the Gram-positive bacterium is selected from the group consisting of Staphylococcus epidermidis, Faecalibacterium sp., Corynebacterium spp., and Clostridium sp. In some aspects, the bacterium is selected from the group consisting of Staphylococcus epidermidis and Corynebacterium spp. In some aspects, the bacterium is S. epidermidis NIHLM087.

In some aspects, the bacterium is selected from the group consisting of: Corynebacterium tuberculostearicum, Corynebacterium accolens, Corynebacterium amycolatum, Corynebacterium aurimucosum, Corynebacterium propinquum, Corynebacterium pseudodiphtheriticum, Corynebacterium granulosum, Cutibacterium acnes, Cutibacterium avidum, Dolosigranulum pigrum, Finegoldia magna, Moraxella catarrhalis, Moraxella catarrhalis, Moraxella nonliquefaciens, Haemophilus influenzae, Haemophilus aegyptius, Rothia mucilaginosa, Streptococcus pyogenes, Streptococcus agalactiae, Streptococcus gordonii, Neisseria lactamica, Neisseria cinerea, Neisseria mucosa, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium breve, Bifidobacterium longum, Veillona parvula, Gardnerella vaginalis, Atopobium vaginae, Prevotella bivia, Mobiluncus mulieris, Mageeibacillus indolicus, Prevotella buccalis, Enterococcus faecium, Lactococcus lactis, Ruminococcus gnavus, and Eubacterium limosum. In some aspects, the commensal bacterium is selected from the group consisting of: a bacterium having ATCC accession number 35692, 49725, 49726, 49368, 700975, 700540, 51488, 10700, 25564, 51277, 11827, 25577, 49753, 51524, 29328, 25238, 25240, 19976, 51907, 11116, 25296, 19615, 12344, BAA-611, 13813, 10558, 23970, 14685, 19696, 33820, 25258, 19992, 55195, 4356, 33200, 7469, 393, 7995D-5, 23272, 11741, 15700, 15697, 10790, 17745, 14018, BAA-55, 29303, 35243, BAA-2120, 35310, 19434, 19435, 29149, and 8486. In some aspects, the commensal bacterium is selected from the group consisting of: Lactobacillus casei, Lactococcus lactis, Streptococcus gordonii, Lactobacillus crispatus, Lactobacillus iners, Cutibacterium acnes, Streptococcus agalactiae, Ruminococcus gnavus, Neisseria lactamica, Bifidobacterium breve, and Bifidobacterium longum. In some aspects, the commensal bacterium is selected from the group consisting of: a bacterium having ATCC accession number 393, 19435, 35105, 33820, 55195, 6919, 13813, 23970, 15700, and 15707, and a bacterium having an accession number JCM6515.

In some aspects, wherein the administration is via a route selected from the group consisting of topical, enteral, and inhalation. In some aspects, the route is topical. In some aspects, the route is enteral.

In some aspects, the protein or peptide is associated with an infection. In some aspects, the infection is selected from the group consisting of a viral infection, a parasitic infection, a bacterial infection, or a fungal infection. In some aspects, the infection occurs at or is otherwise associated with a mucosal boundary of the host. In some aspects, the non-native protein or peptide is derived from a virus, a parasite, a bacterium, or a fungus associated with the infection. In some aspects, the non-native protein or peptide is derived from influenza, HSV, HIV, or SARS-Cov-2. In some aspects, the non-native protein or peptide is selected from the group consisting of: NP366-374, NP306-322, NA177-193, M2 ectodomain, HA2 stem-HA2 12-63, HA2 stem-HA2 76-130, gB glycoprotein, gd glycoprotein, gB glycoprotein 498-505, SARS-Cov2 Spike protein, HIV-gp120, HIV-gp41, HIV V1V2 apex, HIV V3 loop, HIV CD4 binding site, gp120/gp41 interface, gp120 silent face, and HIV membrane-proximal external region (MPER).

In some aspects, the protein or peptide is associated with an autoimmune disorder.

In some aspects, the protein or peptide is associated with a proliferative disorder. In some aspects, the proliferative disorder is cancer. In some aspects, the cancer is selected from melanoma, basal cell carcinoma, squamous cell carcinoma, testicular cancer, sarcoma, and prostate cancer. In some aspects, the cancer is melanoma. In some aspects, the non-native protein or peptide is derived from a melanocyte-specific antigen selected from the group consisting of PMEL, TRP2 and MART-1.

In some aspects, the non-native protein or peptide comprises a neoantigen, wherein the neoantigen comprises at least one mutation that makes the non-native protein or peptide distinct from a protein or peptide encoded by a wild-type gene of the host. In some aspects, wherein the neoantigen is selected from the group consisting of: Ints11, Kif18 bp, T3 sarcoma neoantigens, and a neoantigen expressed by the TRAMPC2 prostate cancer cell line.

In some aspects, the fusion protein further comprises a signal sequence peptide. In some aspects, the signal sequence peptide directs tethering of the fusion protein to a cell wall of the bacterium following expression. In some aspects, the signal sequence peptide that directs secretion comprises a tat signal sequence peptide. In some aspects, the tat signal sequence peptide comprises an S. aureus derived signal sequence peptide. In some aspects, the signal sequence peptide that directs secretion comprises a sec signal sequence peptide. In some aspects, the sec signal sequence peptide comprises an S. epidermidis derived signal sequence peptide. In some aspects, the S. epidermidis derived signal sequence peptide is derived from predicted sec-secreted S. epidermidis protein (gene locus HMPREF9993_06668).

In some aspects, the fusion protein further comprises an antigen-presenting cell (APC) targeting moiety, optionally wherein the APC targeting moiety comprises a CD11b or a MHC II targeting moiety. In some aspects, the APC targeting moiety comprises a nanobody (VHH) antibody binding domain, optionally wherein the VHH antibody binding domain comprises the sequence

(SEQ ID NO: 33)
QVQLQESGGGLVQAGDSLRLSCAASGRTFSRGVMGWFRRAPGKEREFVA
IFSGSSWSGRSTYYSDSVKGRFTISRDNAKNTVYLQMNGLKPEDTAVYY
CAAGYPEAYSAYGRESTYDYWGQGTQVTVSSGG
or
(SEQ ID NO: 34)
QVQLQESGGGLVQAGGSHNLSCTASGITFSSLAMGWFRQTPGKEREFVA
NIMRSGSSVFYADSVRGRFTISRDNAKNTAHLQMNSLKPEDTAVYFCAA
TRGAWPAEYWGQGTQVTVSSGG.

In some aspects, the bacterium is engineered to express a fusion protein comprising the protein or peptide and a native bacterial protein or portion thereof. In some aspects, the protein or peptide is fused to the N-terminus or the C-terminus of the native bacterial protein or portion thereof. In some aspects, the bacterium is formulated for administration in combination with a high-complexity defined microbial community.

In some aspects, the host is a mammal. In some aspects, the mammal is a human.

Also provided herein is a composition comprising a live, recombinant commensal bacterium, wherein the bacterium is engineered to express (a) a first non-native protein or peptide, wherein the first non-native protein or peptide is engineered to elicit a CD4+ T cell response, and (b) a second non-native protein or peptide, wherein the second non-native protein or peptide is engineered to elicit a CD8+ cytotoxic T cell response. In some aspects, the first non-native protein or peptide and the second non-native protein or peptide are each derived from a shared antigen. In some aspects, the first non-native protein or peptide and the second non-native protein or peptide derived from the shared antigen comprise different amino acid sequences. In some aspects, the first non-native protein or peptide and the second non-native protein or peptide are each derived from a different antigen. In some aspects, the colonization of the native host niche is persistent or transient. In some aspects, the native host niche is persistently colonized, and wherein colonization is for at least 60 days, at least 112 days, at least 178 days, at least 1 year, at least 2 years, or at least 5 years. In some aspects, the native host niche is persistently colonized, and wherein colonization is for at least 180 days. In some aspects, the persistent colonization provides a persistent antigen source, optionally wherein the antigen stimulates an antigen-specific T cell population and produces a persistent antigen-specific T cell population. In some aspects, the native host niche is transiently colonized, and wherein colonization is for 1 day to 60 days. In some aspects, the native host niche is transiently colonized, and wherein colonization is for 3.5 days to 60 days. In some aspects, the native host niche is transiently colonized, and wherein colonization is for 7 days to 28 days. In some aspects, colonization is determined by polymerase chain reaction or colony forming assay performed on a sample obtained from the host after 1 day, 3.5 days, 7 days, 14 days, 28 days, or 60 days after administration to the host.

In some aspects, administration results in interaction of the bacterium with a native immune system partner cell. In some aspects, the native immune system partner cell is an antigen-presenting cell. In some aspects, the antigen-presenting cell is selected from the group consisting of a dendritic cell, a macrophage, a B-cell, and an intestinal epithelial cell. In some aspects, the native host niche is selected from the group consisting of the gastrointestinal tract, respiratory tract, urogenital tract, and skin. In some aspects, the non-native protein or peptide is a host protein or peptide.

In some aspects, the bacterium is a Gram-negative bacterium. In some aspects, the Gram-negative bacterium is selected from the group consisting of Bacteroides thetaiotaomicron, Helicobacter hepaticus and Parabacteroides sp.

In some aspects, the bacterium is a Gram-positive bacterium. In some aspects, the Gram-positive bacterium is selected from the group consisting of Staphylococcus epidermidis, Faecalibacterium sp., Corynebacterium spp., Eubacterium limosum, Ruminococcaceae bacterium cv2, Clostridium sp., Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus AGR2154, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Ruminococcus bromii L2-63, Firmicutes bacterium ASF500, Firmicutes bacterium ASF500, Bifidobacterium animalis subsp. lactis ATCC 27673, and Bifidobacterium breve UCC2003. In some aspects, the Gram-positive bacterium is selected from the group consisting of Staphylococcus epidermidis, Faecalibacterium sp., Corynebacterium spp., and Clostridium sp. In some aspects, the bacterium is selected from the group consisting of Staphylococcus epidermidis and Corynebacterium spp. In some aspects, the bacterium is S. epidermidis NIHLM087.

In some aspects, the bacterium is selected from the group consisting of: Corynebacterium tuberculostearicum, Corynebacterium accolens, Corynebacterium amycolatum, Corynebacterium aurimucosum, Corynebacterium propinquum, Corynebacterium pseudodiphtheriticum, Corynebacterium granulosum, Cutibacterium acnes, Cutibacterium avidum, Dolosigranulum pigrum, Finegoldia magna, Moraxella catarrhalis, Moraxella nonliquefaciens, Haemophilus influenzae, Haemophilus aegyptius, Rothia mucilaginosa, Streptococcus pyogenes, Streptococcus agalactiae, Streptococcus gordonii, Neisseria lactamica, Neisseria cinerea, Neisseria mucosa, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium breve, Bifidobacterium longum, Veillonella parvula, Gardnerella vaginalis, Atopobium vaginae, Prevotella bivia, Mobiluncus mulieris, Mageeibacillus indolicus, Prevotella buccalis, Enterococcus faecium, Lactococcus lactis, Ruminococcus gnavus, and Eubacterium limosum. In some aspects, the commensal bacterium is selected from the group consisting of: a bacterium having ATCC accession number 35692, 49725, 49726, 49368, 700975, 700540, 51488, 10700, 25564, 51277, 11827, 25577, 49753, 51524, 29328, 25238, 25240, 19976, 51907, 11116, 25296, 19615, 12344, BAA-611, 13813, 10558, 23970, 14685, 19696, 33820, 25258, 19992, 55195, 4356, 33200, 7469, 393, 7995D-5, 23272, 11741, 15700, 15697, 10790, 17745, 14018, BAA-55, 29303, 35243, BAA-2120, 35310, 19434, 19435, 29149, and 8486. In some aspects, the commensal bacterium is selected from the group consisting of: Lactobacillus casei, Lactococcus lactis, Streptococcus gordonii, Lactobacillus crispatus, Lactobacillus iners, Cutibacterium acnes, Streptococcus agalactiae, Ruminococcus gnavus, Neisseria lactamica, Bifidobacterium breve, and Bifidobacterium longum. In some aspects, the commensal bacterium is selected from the group consisting of: a bacterium having ATCC accession number 393, 19435, 35105, 33820, 55195, 6919, 13813, 23970, 15700, and 15707, and a bacterium having an accession number JCM6515.

In some aspects, wherein the administration is via a route selected from the group consisting of topical, enteral, and inhalation. In some aspects, the route is topical. In some aspects, the route is enteral.

In some aspects, the protein or peptide is associated with an infection. In some aspects, the infection is selected from the group consisting of a viral infection, a parasitic infection, a bacterial infection, or a fungal infection. In some aspects, the infection occurs at or is otherwise associated with a mucosal boundary of the host. In some aspects, the non-native protein or peptide is derived from a virus, a parasite, a bacterium, or a fungus associated with the infection. In some aspects, the non-native protein or peptide is derived from influenza, HSV, HIV, or SARS-Cov-2. In some aspects, the non-native protein or peptide is selected from the group consisting of: NP366-374, NP306-322, NA177-193, M2 ectodomain, HA2 stem-HA2 12-63, HA2 stem-HA2 76-130, gB glycoprotein, gd glycoprotein, gB glycoprotein 498-505, SARS-Cov2 Spike protein, HIV-gp120, HIV-gp41, HIV V1V2 apex, HIV V3 loop, HIV CD4 binding site, gp120/gp41 interface, gp120 silent face, and HIV membrane-proximal external region (MPER).

In some aspects, the protein or peptide is associated with an autoimmune disorder.

In some aspects, the protein or peptide is associated with a proliferative disorder. In some aspects, the proliferative disorder is cancer. In some aspects, the cancer is selected from melanoma, basal cell carcinoma, squamous cell carcinoma, testicular cancer, sarcoma, and prostate cancer. In some aspects, the cancer is melanoma. In some aspects, the non-native protein or peptide is derived from a melanocyte-specific antigen selected from the group consisting of PMEL, TRP2 and MART-1.

In some aspects, the non-native protein or peptide comprises a neoantigen, wherein the neoantigen comprises at least one mutation that makes the non-native protein or peptide distinct from a protein or peptide encoded by a wild-type gene of the host. In some aspects, wherein the neoantigen is selected from the group consisting of: Ints11, Kif18 bp, T3 sarcoma neoantigens, and a neoantigen expressed by the TRAMPC2 prostate cancer cell line.

In some aspects, the fusion protein further comprises a signal sequence peptide. In some aspects, the signal sequence peptide directs tethering of the fusion protein to a cell wall of the bacterium following expression. In some aspects, the signal sequence peptide that directs secretion comprises a tat signal sequence peptide. In some aspects, the tat signal sequence peptide comprises an S. aureus derived signal sequence peptide. In some aspects, the signal sequence peptide that directs secretion comprises a sec signal sequence peptide. In some aspects, the sec signal sequence peptide comprises an S. epidermidis derived signal sequence peptide. In some aspects, the S. epidermidis derived signal sequence peptide is derived from predicted sec-secreted S. epidermidis protein (gene locus HMPREF9993_06668).

In some aspects, the fusion protein further comprises an antigen-presenting cell (APC) targeting moiety, optionally wherein the APC targeting moiety comprises a CD11b or a MHC II targeting moiety. In some aspects, the APC targeting moiety comprises a nanobody (VHH) antibody binding domain, optionally wherein the VHH antibody binding domain comprises the sequence

(SEQ ID NO: 33)
QVQLQESGGGLVQAGDSLRLSCAASGRTFSRGVMGWFRRAPGKEREFVA
IFSGSSWSGRSTYYSDSVKGRFTISRDNAKNTVYLQMNGLKPEDTAVYY
CAAGYPEAYSAYGRESTYDYWGQGTQVTVSSGG
or
(SEQ ID NO: 34)
QVQLQESGGGLVQAGGSHNLSCTASGITFSSLAMGWFRQTPGKEREFVA
NIMRSGSSVFYADSVRGRFTISRDNAKNTAHLQMNSLKPEDTAVYFCAA
TRGAWPAEYWGQGTQVTVSSGG.

In some aspects, the bacterium is engineered to express a fusion protein comprising the protein or peptide and a native bacterial protein or portion thereof. In some aspects, the protein or peptide is fused to the N-terminus or the C-terminus of the native bacterial protein or portion thereof. In some aspects, the bacterium is formulated for administration in combination with a high-complexity defined microbial community.

In some aspects, the host is a mammal. In some aspects, the mammal is a human.

Also provided herein is a composition comprising: (a) a first recombinant commensal bacterium engineered to express a first non-native protein or peptide, wherein the first non-native protein or peptide is engineered to elicit a CD4+ T cell response, and (b) a second recombinant commensal bacterium engineered to express a non-native protein or peptide, wherein the second non-native protein or peptide is engineered to elicit a CD8+ cytotoxic T cell response. In some aspects, the first non-native protein or peptide and the second non-native protein or peptide are each derived from a shared antigen. In some aspects, the first non-native protein or peptide and the second non-native protein or peptide derived from the shared antigen comprise different amino acid sequences. In some aspects, the first non-native protein or peptide and the second non-native protein or peptide are each derived from a different antigen. In some aspects, the colonization of the native host niche is persistent or transient. In some aspects, the native host niche is persistently colonized, and wherein colonization is for at least 60 days, at least 112 days, at least 178 days, at least 1 year, at least 2 years, or at least 5 years. In some aspects, the native host niche is persistently colonized, and wherein colonization is for at least 180 days. In some aspects, the persistent colonization provides a persistent antigen source, optionally wherein the antigen stimulates an antigen-specific T cell population and produces a persistent antigen-specific T cell population. In some aspects, the native host niche is transiently colonized, and wherein colonization is for 1 day to 60 days. In some aspects, the native host niche is transiently colonized, and wherein colonization is for 3.5 days to 60 days. In some aspects, the native host niche is transiently colonized, and wherein colonization is for 7 days to 28 days. In some aspects, colonization is determined by polymerase chain reaction or colony forming assay performed on a sample obtained from the host after 1 day, 3.5 days, 7 days, 14 days, 28 days, or 60 days after administration to the host.

In some aspects, administration results in interaction of the bacterium with a native immune system partner cell. In some aspects, the native immune system partner cell is an antigen-presenting cell. In some aspects, the antigen-presenting cell is selected from the group consisting of a dendritic cell, a macrophage, a B-cell, and an intestinal epithelial cell. In some aspects, the native host niche is selected from the group consisting of the gastrointestinal tract, respiratory tract, urogenital tract, and skin. In some aspects, the non-native protein or peptide is a host protein or peptide.

In some aspects, the bacterium is a Gram-negative bacterium. In some aspects, the Gram-negative bacterium is selected from the group consisting of Bacteroides thetaiotaomicron, Helicobacter hepaticus and Parabacteroides sp.

In some aspects, the bacterium is a Gram-positive bacterium. In some aspects, the Gram-positive bacterium is selected from the group consisting of Staphylococcus epidermidis, Faecalibacterium sp., Corynebacterium spp., Eubacterium limosum, Ruminococcaceae bacterium cv2, Clostridium sp., Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus AGR2154, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Ruminococcus bromii L2-63, Firmicutes bacterium ASF500, Firmicutes bacterium ASF500, Bifidobacterium animalis subsp. lactis ATCC 27673, and Bifidobacterium breve UCC2003. In some aspects, the Gram-positive bacterium is selected from the group consisting of Staphylococcus epidermidis, Faecalibacterium sp., Corynebacterium spp., and Clostridium sp. In some aspects, the bacterium is selected from the group consisting of Staphylococcus epidermidis and Corynebacterium spp. In some aspects, the bacterium is S. epidermidis NIHLM087.

In some aspects, the bacterium is selected from the group consisting of: Corynebacterium tuberculostearicum, Corynebacterium accolens, Corynebacterium amycolatum, Corynebacterium aurimucosum, Corynebacterium propinquum, Corynebacterium pseudodiphtheriticum, Corynebacterium granulosum, Cutibacterium acnes, Cutibacterium avidum, Dolosigranulum pigrum, Finegoldia magna, Moraxella catarrhalis, Moraxella nonliquefaciens, Haemophilus influenzae, Haemophilus aegyptius, Rothia mucilaginosa, Streptococcus pyogenes, Streptococcus agalactiae, Streptococcus gordonii, Neisseria lactamica, Neisseria cinerea, Neisseria mucosa, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium breve, Bifidobacterium longum, Veillonella parvula, Gardnerella vaginalis, Atopobium vaginae, Prevotella bivia, Mobiluncus mulieris, Mageeibacillus indolicus, Prevotella buccalis, Enterococcus faecium, Lactococcus lactis, Ruminococcus gnavus, and Eubacterium limosum. In some aspects, the commensal bacterium is selected from the group consisting of: a bacterium having ATCC accession number 35692, 49725, 49726, 49368, 700975, 700540, 51488, 10700, 25564, 51277, 11827, 25577, 49753, 51524, 29328, 25238, 25240, 19976, 51907, 11116, 25296, 19615, 12344, BAA-611, 13813, 10558, 23970, 14685, 19696, 33820, 25258, 19992, 55195, 4356, 33200, 7469, 393, 7995D-5, 23272, 11741, 15700, 15697, 10790, 17745, 14018, BAA-55, 29303, 35243, BAA-2120, 35310, 19434, 19435, 29149, and 8486. In some aspects, the commensal bacterium is selected from the group consisting of: Lactobacillus casei, Lactococcus lactis, Streptococcus gordonii, Lactobacillus crispatus, Lactobacillus iners, Cutibacterium acnes, Streptococcus agalactiae, Ruminococcus gnavus, Neisseria lactamica, Bifidobacterium breve, and Bifidobacterium longum. In some aspects, the commensal bacterium is selected from the group consisting of: a bacterium having ATCC accession number 393, 19435, 35105, 33820, 55195, 6919, 13813, 23970, 15700, and 15707, and a bacterium having an accession number JCM6515.

In some aspects, wherein the administration is via a route selected from the group consisting of topical, enteral, and inhalation. In some aspects, the route is topical. In some aspects, the route is enteral.

In some aspects, the protein or peptide is associated with an infection. In some aspects, the infection is selected from the group consisting of a viral infection, a parasitic infection, a bacterial infection, or a fungal infection. In some aspects, the infection occurs at or is otherwise associated with a mucosal boundary of the host. In some aspects, the non-native protein or peptide is derived from a virus, a parasite, a bacterium, or a fungus associated with the infection. In some aspects, the non-native protein or peptide is derived from influenza, HSV, HIV, or SARS-Cov-2. In some aspects, the non-native protein or peptide is selected from the group consisting of: NP366-374, NP306-322, NA177-193, M2 ectodomain, HA2 stem-HA2 12-63, HA2 stem-HA2 76-130, gB glycoprotein, gd glycoprotein, gB glycoprotein 498-505, SARS-Cov2 Spike protein, HIV-gp120, HIV-gp41, HIV V1V2 apex, HIV V3 loop, HIV CD4 binding site, gp120/gp41 interface, gp120 silent face, and HIV membrane-proximal external region (MPER).

In some aspects, the protein or peptide is associated with an autoimmune disorder.

In some aspects, the protein or peptide is associated with a proliferative disorder. In some aspects, the proliferative disorder is cancer. In some aspects, the cancer is selected from melanoma, basal cell carcinoma, squamous cell carcinoma, testicular cancer, sarcoma, and prostate cancer. In some aspects, the cancer is melanoma. In some aspects, the non-native protein or peptide is derived from a melanocyte-specific antigen selected from the group consisting of PMEL, TRP2 and MART-1.

In some aspects, the non-native protein or peptide comprises a neoantigen, wherein the neoantigen comprises at least one mutation that makes the non-native protein or peptide distinct from a protein or peptide encoded by a wild-type gene of the host. In some aspects, wherein the neoantigen is selected from the group consisting of: Ints11, Kif18 bp, T3 sarcoma neoantigens, and a neoantigen expressed by the TRAMPC2 prostate cancer cell line.

In some aspects, the fusion protein further comprises a signal sequence peptide. In some aspects, the signal sequence peptide directs tethering of the fusion protein to a cell wall of the bacterium following expression. In some aspects, the signal sequence peptide that directs secretion comprises a tat signal sequence peptide. In some aspects, the tat signal sequence peptide comprises an S. aureus derived signal sequence peptide. In some aspects, the signal sequence peptide that directs secretion comprises a sec signal sequence peptide. In some aspects, the sec signal sequence peptide comprises an S. epidermidis derived signal sequence peptide. In some aspects, the S. epidermidis derived signal sequence peptide is derived from predicted sec-secreted S. epidermidis protein (gene locus HMPREF9993_06668).

In some aspects, the fusion protein further comprises an antigen-presenting cell (APC) targeting moiety, optionally wherein the APC targeting moiety comprises a CD11b or a MHC II targeting moiety. In some aspects, the APC targeting moiety comprises a nanobody (VHH) antibody binding domain, optionally wherein the VHH antibody binding domain comprises the sequence of SEQ ID NO:33 or SEQ ID NO:34.

In some aspects, the bacterium is engineered to express a fusion protein comprising the protein or peptide and a native bacterial protein or portion thereof. In some aspects, the protein or peptide is fused to the N-terminus or the C-terminus of the native bacterial protein or portion thereof. In some aspects, the bacterium is formulated for administration in combination with a high-complexity defined microbial community.

In some aspects, the host is a mammal. In some aspects, the mammal is a human.

Also provided herein is a composition comprising a live, recombinant commensal bacterium, wherein the bacterium is engineered to express a fusion protein comprising a non-native protein or peptide, wherein the non-native protein or peptide is associated with an infection, wherein upon administration of the bacterium to the host resulting in colonization of a native host niche by the bacterium, the host mounts an adaptive immune response to the non-native protein or peptide. In some aspects, the adaptive immune response is a T cell response or a B cell response. In some aspects, the colonization of the native host niche is persistent or transient. In some aspects, the native host niche is persistently colonized, and wherein colonization is for at least 60 days, at least 112 days, at least 178 days, at least 1 year, at least 2 years, or at least 5 years. In some aspects, the native host niche is persistently colonized, and wherein colonization is for at least 180 days. In some aspects, the persistent colonization provides a persistent antigen source, optionally wherein the antigen stimulates an antigen-specific T cell population and produces a persistent antigen-specific T cell population. In some aspects, the native host niche is transiently colonized, and wherein colonization is for 1 day to 60 days. In some aspects, the native host niche is transiently colonized, and wherein colonization is for 3.5 days to 60 days. In some aspects, the native host niche is transiently colonized, and wherein colonization is for 7 days to 28 days. In some aspects, colonization is determined by polymerase chain reaction or colony forming assay performed on a sample obtained from the host after 1 day, 3.5 days, 7 days, 14 days, 28 days, or 60 days after administration to the host.

In some aspects, administration results in interaction of the bacterium with a native immune system partner cell. In some aspects, the native immune system partner cell is an antigen-presenting cell. In some aspects, the antigen-presenting cell is selected from the group consisting of a dendritic cell, a macrophage, a B-cell, and an intestinal epithelial cell. In some aspects, the native host niche is selected from the group consisting of the gastrointestinal tract, respiratory tract, urogenital tract, and skin. In some aspects, the non-native protein or peptide is a host protein or peptide.

In some aspects, the bacterium is a Gram-negative bacterium. In some aspects, the Gram-negative bacterium is selected from the group consisting of Bacteroides thetaiotaomicron, Helicobacter hepaticus and Parabacteroides sp.

In some aspects, the bacterium is a Gram-positive bacterium. In some aspects, the Gram-positive bacterium is selected from the group consisting of Staphylococcus epidermidis, Faecalibacterium sp., Corynebacterium spp., Eubacterium limosum, Ruminococcaceae bacterium cv2, Clostridium sp., Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus AGR2154, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Ruminococcus bromii L2-63, Firmicutes bacterium ASF500, Firmicutes bacterium ASF500, Bifidobacterium animalis subsp. lactis ATCC 27673, and Bifidobacterium breve UCC2003. In some aspects, the Gram-positive bacterium is selected from the group consisting of Staphylococcus epidermidis, Faecalibacterium sp., Corynebacterium spp., and Clostridium sp. In some aspects, the bacterium is selected from the group consisting of Staphylococcus epidermidis and Corynebacterium spp. In some aspects, the bacterium is S. epidermidis NIHLM087.

In some aspects, the bacterium is selected from the group consisting of: Corynebacterium tuberculostearicum, Corynebacterium accolens, Corynebacterium amycolatum, Corynebacterium aurimucosum, Corynebacterium propinquum, Corynebacterium pseudodiphtheriticum, Corynebacterium granulosum, Cutibacterium acnes, Cutibacterium avidum, Dolosigranulum pigrum, Finegoldia magna, Moraxella catarrhalis, Moraxella nonliquefaciens, Haemophilus influenzae, Haemophilus aegyptius, Rothia mucilaginosa, Streptococcus pyogenes, Streptococcus agalactiae, Streptococcus gordonii, Neisseria lactamica, Neisseria cinerea, Neisseria mucosa, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium breve, Bifidobacterium longum, Veillonella parvula, Gardnerella vaginalis, Atopobium vaginae, Prevotella bivia, Mobiluncus mulieris, Mageeibacillus indolicus, Prevotella buccalis, Enterococcus faecium, Lactococcus lactis, Ruminococcus gnavus, and Eubacterium limosum. In some aspects, the commensal bacterium is selected from the group consisting of: a bacterium having ATCC accession number 35692, 49725, 49726, 49368, 700975, 700540, 51488, 10700, 25564, 51277, 11827, 25577, 49753, 51524, 29328, 25238, 25240, 19976, 51907, 11116, 25296, 19615, 12344, BAA-611, 13813, 10558, 23970, 14685, 19696, 33820, 25258, 19992, 55195, 4356, 33200, 7469, 393, 7995D-5, 23272, 11741, 15700, 15697, 10790, 17745, 14018, BAA-55, 29303, 35243, BAA-2120, 35310, 19434, 19435, 29149, and 8486. In some aspects, the commensal bacterium is selected from the group consisting of: Lactobacillus casei, Lactococcus lactis, Streptococcus gordonii, Lactobacillus crispatus, Lactobacillus iners, Cutibacterium acnes, Streptococcus agalactiae, Ruminococcus gnavus, Neisseria lactamica, Bifidobacterium breve, and Bifidobacterium longum. In some aspects, the commensal bacterium is selected from the group consisting of: a bacterium having ATCC accession number 393, 19435, 35105, 33820, 55195, 6919, 13813, 23970, 15700, and 15707, and a bacterium having an accession number JCM6515.

In some aspects, wherein the administration is via a route selected from the group consisting of topical, enteral, and inhalation. In some aspects, the route is topical. In some aspects, the route is enteral.

In some aspects, the protein or peptide is associated with an infection. In some aspects, the infection is selected from the group consisting of a viral infection, a parasitic infection, a bacterial infection, or a fungal infection. In some aspects, the infection occurs at or is otherwise associated with a mucosal boundary of the host. In some aspects, the non-native protein or peptide is derived from a virus, a parasite, a bacterium, or a fungus associated with the infection. In some aspects, the non-native protein or peptide is derived from influenza, HSV, HIV, or SARS-Cov-2. In some aspects, the non-native protein or peptide is selected from the group consisting of: NP366-374, NP306-322, NA177-193, M2 ectodomain, HA2 stem-HA2 12-63, HA2 stem-HA2 76-130, gB glycoprotein, gd glycoprotein, gB glycoprotein 498-505, SARS-Cov2 Spike protein, HIV-gp120, HIV-gp41, HIV V1V2 apex, HIV V3 loop, HIV CD4 binding site, gp120/gp41 interface, gp120 silent face, and HIV membrane-proximal external region (MPER).

In some aspects, the protein or peptide is associated with an autoimmune disorder.

In some aspects, the protein or peptide is associated with a proliferative disorder. In some aspects, the proliferative disorder is cancer. In some aspects, the cancer is selected from melanoma, basal cell carcinoma, squamous cell carcinoma, testicular cancer, sarcoma, and prostate cancer. In some aspects, the cancer is melanoma. In some aspects, the non-native protein or peptide is derived from a melanocyte-specific antigen selected from the group consisting of PMEL, TRP2 and MART-1.

In some aspects, the non-native protein or peptide comprises a neoantigen, wherein the neoantigen comprises at least one mutation that makes the non-native protein or peptide distinct from a protein or peptide encoded by a wild-type gene of the host. In some aspects, wherein the neoantigen is selected from the group consisting of: Ints11, Kif18 bp, T3 sarcoma neoantigens, and a neoantigen expressed by the TRAMPC2 prostate cancer cell line.

In some aspects, the fusion protein further comprises a signal sequence peptide. In some aspects, the signal sequence peptide directs tethering of the fusion protein to a cell wall of the bacterium following expression. In some aspects, the signal sequence peptide that directs secretion comprises a tat signal sequence peptide. In some aspects, the tat signal sequence peptide comprises an S. aureus derived signal sequence peptide. In some aspects, the signal sequence peptide that directs secretion comprises a sec signal sequence peptide. In some aspects, the sec signal sequence peptide comprises an S. epidermidis derived signal sequence peptide. In some aspects, the S. epidermidis derived signal sequence peptide is derived from predicted sec-secreted S. epidermidis protein (gene locus HMPREF9993_06668).

In some aspects, the fusion protein further comprises an antigen-presenting cell (APC) targeting moiety, optionally wherein the APC targeting moiety comprises a CD11b or a MHC II targeting moiety. In some aspects, the APC targeting moiety comprises a nanobody (VHH) antibody binding domain, optionally wherein the VHH antibody binding domain comprises the sequence of SEQ ID NO:33 or SEQ ID NO:34.

In some aspects, the bacterium is engineered to express a fusion protein comprising the protein or peptide and a native bacterial protein or portion thereof. In some aspects, the protein or peptide is fused to the N-terminus or the C-terminus of the native bacterial protein or portion thereof. In some aspects, the bacterium is formulated for administration in combination with a high-complexity defined microbial community.

In some aspects, the host is a mammal. In some aspects, the mammal is a human.

Also provided herein is a composition comprising a polynucleotide used to engineer any of the live, recombinant commensal bacteria described above.

Also provided herein is a method for administering a generating an antigen-presenting cell displaying an antigen derived from a non-native protein or peptide, comprising: administering any of the recombinant commensal bacteria described above to a subject, wherein the administration results in colonization of the native host niche by the bacterium, internalization of the bacterium or the non-native protein or peptide by an antigen-presenting cell, and presentation of the antigen by the antigen-presenting cell. In some aspects, the colonization of the native host niche is persistent or transient. In some aspects, the native host niche is persistently colonized, and wherein colonization is for at least 60 days, at least 112 days, at least 178 days, at least 1 year, at least 2 years, or at least 5 years. In some aspects, the native host niche is persistently colonized, and wherein colonization is for at least 180 days. In some aspects, the persistent colonization provides a persistent antigen source, optionally wherein the antigen stimulates an antigen-specific T cell population and produces a persistent antigen-specific T cell population. In some aspects, the native host niche is transiently colonized, and wherein colonization is for 1 day to 60 days. In some aspects, the native host niche is transiently colonized, and wherein colonization is for 3.5 days to 60 days. In some aspects, the native host niche is transiently colonized, and wherein colonization is for 7 days to 28 days. In some aspects, colonization is determined by polymerase chain reaction or colony forming assay performed on a sample obtained from the host after 1 day, 3.5 days, 7 days, 14 days, 28 days, or 60 days after administration to the host.

In some aspects, the administration results in interaction of the bacterium with a native immune system partner cell. In some aspects, wherein the native immune system partner cell is the antigen-presenting cell. In some aspects, the antigen-presenting cell is selected from the group consisting of a dendritic cell, a macrophage, a B-Cell, and an intestinal epithelial cell. In some aspects, the native host niche is selected from the group consisting of the gastrointestinal tract, respiratory tract, urogenital tract, and skin. In some aspects, the presentation is within an MHC II complex. In some aspects, the presentation is within an MHC I complex.

In some aspects, the bacterium is administered in combination with a high-complexity defined microbial community. In some aspects, the host is a mammal. In some aspects, the mammal is a human.

In some aspects, the method comprises (a) administering a first recombinant commensal bacterium engineered to express a first antigenic peptide comprising the non-native protein or peptide, wherein the first antigenic peptide is engineered to elicit a CD4+ T cell response, and (b) administering a second recombinant commensal bacterium engineered to express a second antigenic peptide comprising the non-native protein or peptide, wherein the second antigenic peptide is engineered to elicit a CD8+ cytotoxic T cell response. In some aspects, the first antigenic peptide comprises a signal sequence peptide that directs secretion of the first antigenic peptide from the bacterium following expression. In some aspects, the second antigenic peptide a signal sequence peptide that directs covalent attachment of the first antigenic peptide to a cell wall of the bacterium following expression.

Also provided herein is a method for generating a T cell response in a subject, comprising: administering any of the recombinant commensal bacteria described above to the subject, wherein the administration results in colonization of a native host niche by the bacterium and generation of the T cell response, wherein the T cell response is to an antigen derived from the non-native protein or peptide. In some aspects, the colonization of the native host niche is persistent or transient. In some aspects, the native host niche is persistently colonized, and wherein colonization is for at least 60 days, at least 112 days, at least 178 days, at least 1 year, at least 2 years, or at least 5 years. In some aspects, the native host niche is persistently colonized, and wherein colonization is for at least 180 days. In some aspects, the persistent colonization provides a persistent antigen source, optionally wherein the antigen stimulates an antigen-specific T cell population and produces a persistent antigen-specific T cell population. In some aspects, the native host niche is transiently colonized, and wherein colonization is for 1 day to 60 days. In some aspects, the native host niche is transiently colonized, and wherein colonization is for 3.5 days to 60 days. In some aspects, the native host niche is transiently colonized, and wherein colonization is for 7 days to 28 days. In some aspects, colonization is determined by polymerase chain reaction or colony forming assay performed on a sample obtained from the host after 1 day, 3.5 days, 7 days, 14 days, 28 days, or 60 days after administration to the host.

In some aspects, the administration is via a route selected from the group consisting of topical, enteral, parenteral and inhalation. In some aspects, the route is topical. In some aspects, the route is enteral.

In some aspects, the T cell response comprises a CD4+T-helper response, a CD8+ cytotoxic T cell response, or a CD4+T helper response and a CD8+ cytotoxic T cell response. In some aspects, the CD4+T-helper response is a T_{H}1 response, a T_{H}2 response, a T_{H}17 response, or a combination thereof. In some aspects, the CD4+T-helper response is a T_{H}1 response. In some aspects, the CD4+T-helper response is a T_{H}2 response. In some aspects, the T cell response comprises a T_{reg} response.

In some aspects, the bacterium is administered in combination with a high-complexity defined microbial community. In some aspects, the host is a mammal. In some aspects, the mammal is a human.

In some aspects, the method comprises (a) administering a first recombinant commensal bacterium engineered to express a first antigenic peptide comprising the non-native protein or peptide, wherein the first antigenic peptide is engineered to elicit a CD4+ T cell response, and (b) administering a second recombinant commensal bacterium engineered to express a second antigenic peptide comprising the non-native protein or peptide, wherein the second antigenic peptide is engineered to elicit a CD8+ cytotoxic T cell response. In some aspects, the first antigenic peptide comprises a signal sequence peptide that directs secretion of the first antigenic peptide from the bacterium following expression. In some aspects, the second antigenic peptide a signal sequence peptide that directs covalent attachment of the first antigenic peptide to a cell wall of the bacterium following expression.

Also provided herein is a method of treating a disease or condition in a subject, comprising: administering any of the recombinant commensal bacteria described above to the subject, wherein the administration results in colonization of a native host niche by the bacterium and generation of a T cell response, wherein the T cell response is to an antigen derived from the non-native protein or peptide, and wherein the T cell response treats the disease or condition in the subject. In some aspects, the colonization of the native host niche is persistent or transient. In some aspects, the native host niche is persistently colonized, and wherein colonization is for at least 60 days, at least 112 days, at least 178 days, at least 1 year, at least 2 years, or at least 5 years. In some aspects, the native host niche is persistently colonized, and wherein colonization is for at least 180 days. In some aspects, the persistent colonization provides a persistent antigen source, optionally wherein the antigen stimulates an antigen-specific T cell population and produces a persistent antigen-specific T cell population. In some aspects, the native host niche is transiently colonized, and wherein colonization is for 1 day to 60 days. In some aspects, the native host niche is transiently colonized, and wherein colonization is for 3.5 days to 60 days. In some aspects, the native host niche is transiently colonized, and wherein colonization is for 7 days to 28 days. In some aspects, colonization is determined by polymerase chain reaction or colony forming assay performed on a sample obtained from the host after 1 day, 3.5 days, 7 days, 14 days, 28 days, or 60 days after administration to the host.

In some aspects, the disease or condition is an infection, a proliferative disorder, or an autoimmune disorder. In some aspects, the infection is selected from the group consisting of a viral infection, a parasitic infection, a bacterial infection, or a fungal infection. In some aspects, the proliferative disorder is cancer. In some aspects, the cancer is selected from melanoma, basal cell carcinoma, squamous cell carcinoma, testicular cancer, cervical cancer, anal cancer and nasopharyngeal cancer. In some aspects, the cancer is melanoma.

In some aspects, the administration is via a route selected from the group consisting of topical, enteral, parenteral and inhalation. In some aspects, the route is topical. In some aspects, the bacterium is S. epidermidis.

In some aspects, the disease is cancer. In some aspects, the cancer is melanoma. In some aspects, the non-native protein or peptide is selected from the group consisting of a melanocyte-specific antigen and a testis cancer antigen, optionally wherein the melanocyte-specific antigen is selected from the group consisting of PMEL, TRP2 and MART-1 and optionally wherein the testis cancer antigen is selected from the group consisting of NY-ESO and MAGE-A. In some aspects, the non-native protein or peptide comprises a neoantigen, wherein the neoantigen comprises at least one mutation that makes the non-native protein or peptide distinct from a protein or peptide encoded by a wild-type gene of the host.

In some aspects, the bacterium is administered in combination with a high-complexity defined microbial community. In some aspects, the host is a mammal. In some aspects, the mammal is a human.

In some aspects, the method comprises (a) administering a first recombinant commensal bacterium engineered to express a first antigenic peptide comprising the non-native protein or peptide, wherein the first antigenic peptide is engineered to elicit a CD4+ T cell response, and (b) administering a second recombinant commensal bacterium engineered to express a second antigenic peptide comprising the non-native protein or peptide, wherein the second antigenic peptide is engineered to elicit a CD8+ cytotoxic T cell response. In some aspects, the first antigenic peptide comprises a signal sequence peptide that directs secretion of the first antigenic peptide from the bacterium following expression. In some aspects, the second antigenic peptide a signal sequence peptide that directs covalent attachment of the first antigenic peptide to a cell wall of the bacterium following expression.

In some aspects, the method further comprises co-administering one or more additional agents. In some aspects, the one or more additional agents comprises one or more checkpoint inhibitors.

In some aspects, a distal adaptive immune response is produced. In some aspects, the distal adaptive immune response is distal from the site of administration. In some aspects, the distal adaptive immune response is distal from the native host niche. In some aspects, the distal adaptive immune response comprises an immune response in an organ that is not the organ of the site of administration and/or the native host niche. In some aspects, the site of administration and/or the native host niche comprises skin. In some aspects, the distal adaptive immune response comprises an antitumor response. In some aspects, the antitumor response targets a metastasis.

In some aspects, provided herein is live, recombinant commensal bacterium engineered to express a fusion protein, the fusion protein comprising: (a) a non-native protein or peptide, and (b)(i) a tat signal sequence peptide, a sec signal sequence peptide, or a sortase-derived signal sequence peptide, and/or an antigen-presenting cell (APC) targeting moiety, or (ii) a tat signal sequence peptide, a sec signal sequence peptide, or a sortase-derived signal sequence peptide, wherein administration of the bacterium to the host results in colonization of a native host niche by the bacterium, and generation of an adaptive immune response by the host against the non-native protein or peptide.

In some aspects, the non-native protein or peptide is associated with a host disease or condition selected from the group consisting of: (i) a cancer; (ii) an autoimmune disorder; and (iii) an infection that occurs at or is otherwise associated with a mucosal boundary of the host.

In some aspects the signal sequence peptide: (i) directs tethering of the expressed fusion protein to a cell wall of the bacterium; or (ii) directs secretion of the fusion protein from the bacterium following expression.

In some aspects, the tat signal sequence peptide comprises a sequence derived from fepB of Staphylococcus aureus, the sec signal sequence peptide comprises a sequence derived from predicted sec-secreted Staphylococcus epidermidis protein (gene locus HMPREF9993_06668), or the sortase-derived signal sequence peptide comprises one or more sequences derived from Protein A of S. aureus.

In some aspects, the signal sequence peptide is fused to the N-terminal side of the non-native protein or peptide and the fusion protein comprises a cell-wall spanning peptide domain on the C-terminal side of the non-native protein or peptide.

In some aspects, the APC targeting moiety comprises a CD11b or MHCII targeting moiety.

In some aspects, the native host niche is selected from the group consisting of the gastrointestinal tract, respiratory tract, urogenital tract, and skin.

In some aspects, the adaptive immune response is distal from the site of administration and/or the native host niche. In some aspects, the distal adaptive immune response comprises an immune response in an organ that is not the organ of the site of administration and/or the native host niche, and optionally wherein the site of administration and/or the native host niche comprises skin. In some aspects, the distal adaptive immune response comprises an antitumor response, optionally wherein the antitumor response targets a metastasis.

In some aspects, the colonization of the native host niche is persistent or transient. In some aspects, the native host niche is persistently colonized, and wherein colonization is for at least 60 days, at least 112 days, at least 178 days, at least 180 days, at least 1 year, at least 2 years, or at least 5 years. In some aspects the persistent colonization provides a persistent antigen source, optionally wherein the antigen stimulates an antigen-specific T cell population and produces a persistent antigen-specific T cell population. In some aspects, the native host niche is transiently colonized, and wherein colonization is for 1 day to 60 days, 3.5 days to 60 days, or 7 days to 28 days.

In some aspects, the fusion protein comprises the non-native protein or peptide fused to the N-terminus or the C-terminus of a native bacterial protein or portion thereof.

In some aspects, the bacterium is formulated for administration in combination with a high-complexity defined microbial community.

In some aspects, the live, recombinant commensal bacterium is (i) a Gram-positive bacterium selected from the group consisting of Staphylococcus epidermidis, Faecalibacterium sp., Corynebacterium spp., Eubacterium limosum, Ruminococcaceae bacterium cv2, Clostridium sp., Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Ruminococcus bromii L2-63, Firmicutes bacterium ASF500, Firmicutes bacterium ASF500, Bifidobacterium animalis subsp. lactis ATCC 27673, Bifidobacterium breve, Cutibacterium acnes, Cutibacterium avidum, Dolosigranulum pigrum, Finegoldia magna, Rothia mucilaginosa, Streptococcus pyogenes, Streptococcus agalactiae, Streptococcus gordonii, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium longum, Gardnerella vaginalis, Atopobium vaginae, Mobiluncus mulieris, Mageeibacillus indolicus, Enterococcus faecium, and Lactococcus lactis, and optionally wherein the bacterium is S. epidermidis NIHLM087; or (ii) a Gram-negative bacterium selected from the group consisting of Bacteroides thetaiotaomicron, Helicobacter hepaticus, Parabacteroides sp., Moraxella catarrhalis, Moraxella nonliquefaciens, Haemophilus influenzae, Haemophilus aegyptius, Neisseria lactamica, Neisseria cinerea, Neisseria mucosa, Veillonella parvula, Prevotella bivia, Prevotella buccalis, Gardnerella vaginalis, and Mobiluncus mulieris.

In some aspects, provided herein is a method of treating a disease or condition in a subject, comprising: administering a live, recombinant commensal bacterium engineered to express a heterologous antigen to a subject, wherein the expressed heterologous antigen induces an antigen-specific immune response to treat the disease or condition in the subject. In some aspects, the adaptive immune response to the non-native protein or peptide treats the disease or condition in the subject. In some aspects, the administration is via a route selected from the group consisting of topical, enteral, parenteral and inhalation.

In some aspects, the method further comprises co-administering one or more additional agents, and optionally wherein the one or more additional agents comprises one or more checkpoint inhibitors.

In some aspects, the bacterium is engineered to express (a) a first non-native protein or peptide, wherein the first non-native protein or peptide is engineered to elicit a CD4+ T cell response, and (b) a second non-native protein or peptide, wherein the second non-native protein or peptide is engineered to elicit a CD8+ cytotoxic T cell response, and wherein administration of the bacterium to a host results in colonization of a native host niche by the bacterium.

In some aspects, provided herein is a composition comprising: (a) a first live, recombinant commensal bacterium engineered to express a first non-native protein or peptide, wherein the first non-native protein or peptide is engineered to elicit a CD4+ T cell response, and (b) a second live, recombinant commensal bacterium engineered to express a second non-native protein or peptide, wherein the second non-native protein or peptide is engineered to elicit a CD8+ cytotoxic T cell response, and wherein administration of the composition to a host results in colonization of a native host niche by the first live, recombinant commensal bacterium and the second live, recombinant commensal bacterium.

In some aspects the first non-native protein or peptide and the second non-native protein or peptide are each derived from a shared antigen or a different antigen, and optionally when the first non-native protein or peptide and the second non-native protein or peptide are derived from the shared antigen, the first non-native protein or peptide and the second non-native protein or peptide comprise different amino acid sequences. In some aspects, the first non-native protein or peptide comprises a signal sequence peptide that directs secretion of the non-native protein or peptide from the first live, recombinant commensal bacterium following expression, and/or the second non-native protein or peptide comprises a second signal sequence peptide that directs covalent attachment of the second non-native protein or peptide to a cell wall of the second live, recombinant commensal bacterium following expression.

In some aspects, provided herein is a method of treating a disease or condition in a host, comprising: administering a live, recombinant commensal bacterium, or a composition of the present invention to the host, wherein the elicited CD4+ T cell response and CD8+ cytotoxic T cell response treats the disease or condition in the host.

In some aspects, provided herein is a bacterial surface display system comprising: (a) a fusion protein comprising a cell-surface tethering moiety and a non-native protein or peptide; (b) a bacterium; and (c) a protein or gene encoding the same capable of catalyzing a covalent attachment of the cell-surface tethering moiety to a cell wall protein or outer membrane protein of the bacterium thereby displaying the fusion protein on a bacterial surface.

In some aspects, provided herein is a bacterial surface display system comprising: (a) a fusion protein comprising a cell-surface tethering moiety and a non-native protein or peptide and (b) a bacterium, wherein the fusion protein is covalently attached to a cell wall protein or outer membrane protein via the cell-surface tethering moiety, and wherein the covalent attachment was catalyzed by a protein capable of catalyzing attachment of the cell-surface tethering moiety to the cell wall protein or outer membrane protein of the bacterium.

In some aspects, the cell-surface tethering moiety comprises a Sortase A (SrtA) motif and the protein capable of catalyzing the covalent attachment is a SrtA protein. In some aspects, the SrtA motif and/or the SrtA protein is derived from S. aureus, optionally wherein the SrtA motif comprises the amino acid sequence LPXTG.

In some aspects the fusion protein comprises an antigenic protein or peptide associated with a host disease or condition selected from the group consisting of a proliferative disorder, an autoimmune disorder, and an infection.

In some aspects, administration of the bacterium to a host results in colonization of a native host niche by the bacterium eliciting a T-cell response to the non-native protein or peptide.

In some aspects, the fusion protein further comprises an antigen-presenting cell (APC) targeting moiety, optionally wherein the APC targeting moiety comprises a CD11b or a MHC II targeting moiety.

In some aspects, the bacterium is (i) a Gram-positive bacterium selected from the group consisting of Staphylococcus epidermidis, Faecalibacterium sp., Corynebacterium spp., Eubacterium limosum, Ruminococcaceae bacterium cv2, Clostridium sp., Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Ruminococcus bromii L2-63, Firmicutes bacterium ASF500, Firmicutes bacterium ASF500, Bifidobacterium animalis subsp. lactis ATCC 27673, Bifidobacterium breve, Cutibacterium acnes, Cutibacterium avidum, Dolosigranulum pigrum, Finegoldia magna, Rothia mucilaginosa, Streptococcus pyogenes, Streptococcus agalactiae, Streptococcus gordonii, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium longum, Gardnerella vaginalis, Atopobium vaginae, Mobiluncus mulieris, Mageeibacillus indolicus, Enterococcus faecium, and Lactococcus lactis, and optionally wherein the bacterium is S. epidermidis NIHLM087; or (ii) a Gram-negative bacterium selected from the group consisting of Bacteroides thetaiotaomicron, Helicobacter hepaticus, Parabacteroides sp., Moraxella catarrhalis, Moraxella nonliquefaciens, Haemophilus influenzae, Haemophilus aegyptius, Neisseria lactamica, Neisseria cinerea, Neisseria mucosa, Veillonella parvula, Prevotella bivia, Prevotella buccalis, Gardnerella vaginalis, and Mobiluncus mulieris.

In some aspects, provided herein is a pharmaceutical composition comprising the bacterial surface display system of the present invention, and an excipient. In some aspects, the pharmaceutical composition further comprises a high-complexity defined microbial community.

In some aspects, provided herein is a method of treating a disease or condition in a host, comprising: administering the bacterial surface display system, or pharmaceutical composition of the present invention, to the host, wherein the administration results in colonization of a native host niche in the host by the bacterium, internalization of the bacterium or the non-native protein or peptide by an antigen-presenting cell, presentation of an antigen derived from the non-native protein or peptide by the antigen-presenting cell within an MHC-I or MHC-II complex, and generation of a T-cell response to the antigen, and wherein the T-cell response treats the disease or condition in the host. In some aspects, the colonization of the native host niche is persistent or transient. In some aspects, the native host niche is transiently colonized, and wherein colonization is for 1 day to 60 days, 3.5 days to 60 days, or 7 days to 28 days. In some aspects, the native host niche is selected from the group consisting of the gastrointestinal tract, respiratory tract, urogenital tract, and skin.

In some aspects, the host is a subject. In some aspects, the subject is a human.

### BRIEF DESCRIPTION OF THE DRAWINGS

FIG. 1 is a diagram illustrating an exemplary method for generating a regulatory T cell response to an exogenous antigen expressed by a recombinant bacterial strain of the disclosure.

FIG. 2 is an image of a Western blot analysis demonstrating expression of OVA antigen peptide by Bacteroides thetaiotaomicron engineered to express ovalbumin (OVA) peptide.

FIG. 3A and FIG. 3B are dot plots showing flow cytometry analysis of Nur77 expression in OVA-specific T cells from the spleen of OTII transgenic mice co-cultured for 4 hours with B16-FLT3L stimulated DCs and OVA+B. thetaiotaomicron (FIG. 3B) or WT B. thetaiotaomicron (negative control; FIG. 3A).

FIG. 4A, FIG. 4B, and FIG. 4C are images of Western blot analyses demonstrating expression of myelin oligodendrocyte glycoprotein (MOG) fusion constructs by B. thetaiotaomicron (FIG. 4A), Bacteroides vulgatus (FIG. 4B), and Bacteroides finegoldii (FIG. 4C).

FIG. 5A and FIG. 5B are bar graphs showing flow cytometry data of CD4+ T cell activation (% CD69+ of CD4+ T cells and % CTV-CD44+ of CD4+ T cells, respectively) in in vitro co-cultures comprising antigen presenting cells (APC; splenic dendritic cells), myelin oligodendrocyte glycoprotein (MOG)-specific T cells, and live or autoclaved wild-type B. thetaiotaomicron or various recombinant B. thetaiotaomicron strains engineered to express different MOG35-55 peptide constructs.

FIG. 6 is a graph showing Experimental Autoimmune Encephalomyelitis (EAE) scores of gnotobiotic mice administered with a mixture of B. vulgatus and B. finegoldii expressing wildtype MOG (BVF_WT) or a mixture of B. vulgatus and B. finegoldii expressing MOG fusion constructs (BVF_MOG) two weeks prior to induction of EAE (Day 0).

FIG. 7A, FIG. 7B, and FIG. 7C are bar graphs showing flow cytometry data of CD4+ T cell populations (% Foxp3+ Helios− of CD4+ T cells (FIG. 7A), % IL17+ of CD4+ T cells (FIG. 7B), and % IFNγ+ of CD4+ T cells (FIG. 7C)) at Day 7 in mice administered with a mixture of wild-type B. vulgatus and B. finegoldii (BVF_WT) or a mixture of recombinant B. vulgatus and B. finegoldii engineered to express MOG35-55 fusion constructs (BVF_MOG) two weeks prior to induction of EAE (Day 0).

FIG. 8A and FIG. 8B are graphs showing flow cytometry data of % Nur77+ of CD8+ T cells (FIG. 8A) and % Nur77+ of CD4+ T cells (FIG. 8B) as an indication of T cell activation in in vitro co-cultures comprising APCs, ovalbumin (OVA)-specific T cells isolated from OT-I or OT-II transgenic mice, and various recombinant Staphylococcus epidermidis strains engineered to express different OVA peptide constructs. PBS=Phosphate Buffered Saline (negative control); PMA/Iono=phorbol myristate acetate/ionomycin (positive control).

FIG. 9 is a bar graph showing flow cytometry data of % Nur77+ of CD8+ T cells as an indicator of T cell activation in in vitro co-cultures comprising APCs, PMEL antigen-specific T cells isolated from 8rest transgenic mice, and recombinant Staphylococcus epidermidis strains engineered to express different PMEL antigen constructs. PBS=Phosphate Buffered Saline (negative control); PMA/Iono=phorbol myristate acetate/ionomycin (positive control).

FIG. 10A is a graph showing OVA+B16F0 melanoma tumor weights in mice topically associated with recombinant S. epidermidis engineered to express OVA+/−luciferase either 2 weeks before (“before tumor”) or 1 week after (“after tumor”) subcutaneous or intraperitoneal injection of melanoma cells. FIG. 10B is a graph showing tumor radiance over time of OVA+B16F0 melanoma tumors in mice topically associated with wildtype S. epidermidis (S epi control) or recombinant S. epidermidis engineered to express OVA (S epi OVA), 1 day to 3 days after intraperitoneal injection of OVA+B16F0 melanoma tumors. FIG. 10C is a graph showing tumor radiance in the mice of FIG. 10B 13 days after topical association of wildtype S. epidermidis (S epi control) or recombinant S. epidermidis engineered to express OVA (S epi OVA).

FIG. 11A, FIG. 11B, FIG. 11C, and FIG. 11D are diagrams and data illustrating antigen fusion constructs engineered to be expressed in bacteria. FIG. 11A and FIG. 11B show schematic illustrations of a tat expression system and a sortase expression system, respectively, that control localization of the expressed antigen after transformation into bacteria. FIG. 11C shows schematic illustrations of various constructs for expression of OVA antigen or peptide fragments OT1, OT2 or OT3pep (OVA3pep) with directed localization to the cytosol, cell wall, or secretion. FIG. 11D shows a western blot analysis of proteins extracted from cell pellets or overnight liquid culture supernatants of S. epi-sOVA (secreted OVA) or S. epi-cOVA (cytoplasmic OVA).

FIG. 12A and FIG. 12B are graphs showing flow cytometry analysis of Nur77 expression, a marker for the activation of T cells, in in vitro co-culture experiments. FIG. 12A is a graph showing % Nur77+ cells of OT-I stimulated antigen-specific CD8+ T cells cultured in the presence of splenic dendritic cells and S. epidermidis expressing OVA fusion proteins or peptides or S. epidermidis expressing control peptide. FIG. 12B is a graph showing % Nur77+ cells of OT-II stimulated antigen-specific CD4+ T cells cultured in the presence of splenic dendritic cells and S. epidermidis expressing OVA fusion proteins or peptides or S. epidermidis expressing control peptide.

FIG. 13A is a bar graph showing tumor volumes and FIG. 13B is a bar graph showing tumor weights after 21-23 days of tumor growth in mice inoculated with S. epidermidis engineered to express OVA antigen or control for one week prior to subcutaneous xenograft with OVA-positive B16F10 melanoma cells.

FIG. 14A is a bar graph showing tumor weights in mice inoculated with S. epidermidis expressing secreted OVA (sOVAtat), wall-attached OT1 (wOVApep), both antigen constructs in live bacteria (OVA), or both antigen constructs in heat-killed bacteria (HK OVA) for one week prior to subcutaneous xenograft with OVA-positive B16F10 melanoma cells. Certain groups of mice inoculated with both live bacterial strains were further treated with anti-CD8 antibodies (OVA+aCD8) or anti-T cell receptor (TCR) antibodies (OVA+aTCRb). FIG. 14B and FIG. 14C are graphs showing the number of splenic CD4+ T cells and CD8+ T cells, respectively, in mice topically associated with S. epidermidis engineered to express OVA (S. epi-OVA) and subcutaneously injected with B16-F0-OVA tumors. Control groups were additionally treated with anti-CD8 neutralizing antibody (S.epi-OVA+anti-CD8), or anti-TCRβ neutralizing antibody (S.epi-OVA+anti-TCRb).

FIG. 15A, FIG. 15B, FIG. 15C, FIG. 15D, FIG. 15E, FIG. 15F, FIG. 15G, and FIG. 15H are bar graphs showing the percentage of CD8+ T cells or CD4+ T cells in the draining lymph nodes of mice inoculated with S. epidermidis engineered to express a combination of OVA antigens (S. epi/OVA combo) or control antigen (S. epi control) for one week prior to subcutaneous xenograft of OVA-positive B16-F0 melanoma cells. FIG. 15A, FIG. 15B, and FIG. 15C are graphs showing flow cytometry analysis of the total percentage of CD8+ T cells (FIG. 15A), IFNγ+CD8+ T cells (FIG. 15B), and Tetramer+ CD8+ T cells (FIG. 15C). FIG. 15D and FIG. 15E are graphs showing flow cytometry analysis of the total percentage of CD4+ T cells (FIG. 15D) and IFNγ+CD4+ T cells (FIG. 15E). FIG. 15F is a graph showing subcutaneous B16-F0-OVA tumor weights on day 21-22 from mice colonized with S. epidermidis engineered to express different versions of OVA (wildtype OVA (OVA); wall-spanning OVA (wOVA); wall-spanning OVA fragment OT1 and secreted OVA fragment OT2 (wOT1+sOT2); or wall-spanning OVA fragment OT2 and secreted OVA fragment OT1 (wOT2+sOT1)). FIG. 15G and FIG. 15H are graphs showing flow cytometry analysis of the percentages of IFNγ+CD4+ T cells and IFNγ+CD8+ T cells, respectively, in tumor-draining inguinal lymph nodes from mice subcutaneously xenografted with B16-F0 OVA tumor cells and colonized with S. epidermidis engineered to express wildtype OVA, wall-spanning OVA fragment OT1 (wOT1); or wall-spanning OVA fragment OT2 (wOT2).

FIG. 16A, FIG. 16B, FIG. 16C, and FIG. 16D are diagrams illustrating strategies for antigen-presenting cell (APC)-targeting. FIG. 16A is a diagram illustrating T cell activation using antigens attached to APC targeting moieties. FIG. 16B, FIG. 16C, and FIG. 16D are diagrams illustrating functional antibody fragments, including a conventional antibody (FIG. 16B), a heavy-chain only antibody (FIG. 16C), and a nanobody/variable heavy chain homodimer (VHH) fragment (FIG. 16D), that can effectively bind antigens.

FIGS. 17A and 17B are schematic diagrams of fusion proteins designed to present influenza A virus (IAV) antigens in recombinant bacteria to induce a T cell response. FIG. 17A shows designs for two constructs designed to induce a CD8+ T cell response to IAV nucleoprotein peptide fragment NP₃₆₆₋₃₇₄, with the bottom construct containing a VHH fragment targeting CD11b on APCs. FIG. 17B shows designs for four constructs to induce a CD4+ T cell response to either IAV NP₃₆₆₋₃₇₄ or IAV neuraminidase fragment NA₁₇₇₋₁₉₃, with the bottom two constructs containing a VHH fragment targeting MHC-II on APCs.

FIG. 18A and FIG. 18B are graphs showing serum anti-OVA immunoglobulin G (IgG) in mice inoculated with S. epidermidis expressing a combination of ovalbumin constructs (OVA combo) at 3 weeks and 5 weeks post-inoculation, respectively.

FIG. 19 is a schematic diagram illustrating six constructs designed to present one of three IAV antigens ((M2e)₄, HA2₇₆₋₁₃₀, or HA2₁₂₋₆₃) in recombinant bacteria to induce a B cell response, with the bottom 3 constructs containing a VHH fragment targeting MHC-II on APCs.

FIG. 20 is an illustration of an experimental workflow to test immunization against IAV using recombinant commensal bacteria in mice. Mice are inoculated with recombinant commensal bacteria engineered to express IAV antigens, infected with IAV, then analyzed for infection, survival, and symptoms of infection.

FIG. 21A is an illustration of an experimental workflow to test immunization against metastatic melanoma. Mice are colonized topically with live S. epidermidis strains engineered to express OVA antigen starting 7 days prior to tumor injection. On day 0, B16-F10-OVA melanoma cells (which express luciferase constitutively) are freshly prepared from growing cultures and injected intravenously into the tail vein. The tumor burden in live mice is monitored 1-2×/week by intraperitoneal luciferin injection followed by bioluminescence imaging with an IVIS Lumina Imager. Mice are sacrificed on day 22. FIG. 21B are schematic diagrams of neoantigen expression constructs and their predicted subcellular localization within S. epidermidis. The wall-attachment and secretion scaffolds are identical to those for wOT1 and sOT1. The neoantigen coding sequence encodes 27-aa pepti-des centered around Obsl1(T1764M) for the wall-attached construct (wB16Ag) or around Ints11(D314N) for the secreted construct (sB16Ag). FIG. 21C is a line graph quantifying tumor radiance/bioluminescence in mice treated according to FIG. 21A, with dots showing the average measurement at each post-tumor injection timepoint. FIG. 21D is a bar graph quantifying tumor radiance/bioluminescence in mice treated according to FIG. 21A on day 15 post-tumor injection with each dot representing the measurement for each individual mouse. FIG. 21E is a diagram illustrating a model of antitumor response induced by engineered commensal bacteria. Antigen-expressing strains of S. epidermidis colonize the skin and induce antigen-presenting cells to stimulate CD8+ or CD4+ T cells, which then traffic to the tumor to restrict tumor growth.

FIG. 22 is a set of representative images of bioluminescence of metastatic tumors in mice topically associated with wild-type S. epidermidis (S. epi-control), S. epidermidis engineered to express wild-type ovalbumin (S. epi-OVA), or S. epidermidis engineered to express a neoantigen (S. epi-neoAg) on day 4 (left panels) or day 15 (right panels) after intravenous tumor injection.

FIG. 23A, FIG. 23B, and FIG. 23C are diagrams illustrating a bacterial surface display system to anchor fusion proteins onto bacteria using Sortase A (SrtA). FIG. 23A is a diagram illustrating heterologous expression of antigens in tractable commensal organisms and a surface display system utilizing SrtA in intractable organisms. FIG. 23B is a diagram illustrating the mechanism by which SrtA anchors non-native proteins onto the cell wall (e.g., S. epidermidis).

FIG. 23C is a diagram illustrating the design of various constructs that, when expressed, can be anchored to a bacterial cell wall using a SrtA surface display system.

FIG. 24A, FIG. 24B, FIG. 24C, and FIG. 24D show the efficacy of engineered S. epidermidis strains on established tumors. FIG. 24A shows the treatment of subcutaneous B16-F0-OVA melanoma with topical association of S. epi-OVApep. The left graph shows blinded caliper measurements (n=10/group, bilateral tumors). The right graph shows Day 21 tumor weights from the same experiment. FIG. 24B shows the treatment of metastatic B16-F10-OVA melanoma with topical association of S. epi-OVApep. The left graph shows tumor burden as quantified by bioluminescence imaging. The right graph shows the frequency of OT-I-specific T cells in the spleen at day 20 by H2-Kb-SIINFEKL tetramer staining. Cells were gated on live CD90.2+ TCRβ+CD8β+ cells. FIG. 24C shows the treatment of subcutaneous B16-F10-OVA melanoma with immune checkpoint blockade after pre-association with S. epi-OVApep. The left graph shows blinded caliper measurements (n=8 mice, bilateral tumors). The top right graph shows the survival curve from this experiment. The bottom right graph shows 14/16 responders initially injected with unilateral tumors that were re-challenged (opposite flank) without receiving any additional treatment. The graph depicts caliper measurements of the re-challenged left flank tumors. FIG. 24D shows the treatment of established B16-F10-OVA melanoma with immune checkpoint blockade and topical S. epi-OVApep. Blinded caliper measurements (n=16 mice, bilateral tumors, 2 experiments pooled). For bar graphs in FIG. 24A, FIG. 24B, FIG. 24C, and FIG. 24D: the Mann-Whitney U test was used to generate P-values. For tumor growth time courses in FIG. 24A, FIG. 24B, FIG. 24C, and FIG. 24D: two-way ANOVA with multiple comparison testing was used.

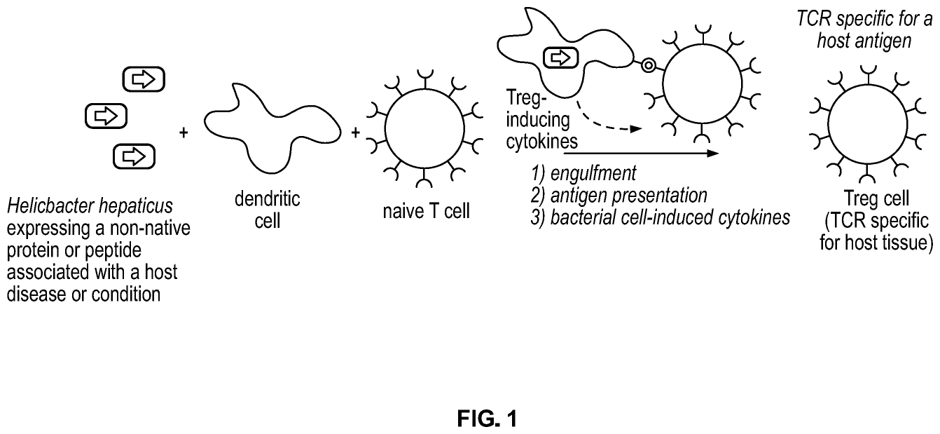

### DETAILED DESCRIPTION

### 1. Definitions

Unless defined otherwise, all technical and scientific terms used herein have the meaning commonly understood by one of ordinary skill in the art to which this disclosure belongs.

The term “a” and “an” as used herein mean “one or more” and include the plural unless the context is appropriate.

As used herein, the term “commensal” means a relationship between two or more organisms. In certain embodiments, commensal refers to a relationship between two or more organisms of different species in which one generally derives some benefit while another is generally unharmed. In certain embodiments, a commensal refers to a relationship between two or more organisms of different species in which one organism derives a benefit from another organism. In certain embodiments, a commensal refers to a relationship between two or more organisms of different species in which a first organism derives a benefit from a second organism and the second organism is unharmed. In certain embodiments, a commensal refers to a symbiotic relationship between two or more organisms. In certain embodiments, a commensal refers to a symbiotic relationship between two or more organisms wherein a first organism derives a benefit from a second organism and the second organism is unharmed. In certain embodiments, a commensal microbe may be one that is normally present as a non-pathogenic member of a host gut microbiome, a host skin microbiome, a host mucosal microbiome, or other host niche microbiome.

As used herein, the term “bacteria” includes both singular and plural forms, such as a bacterium (single bacterial cell) and bacteria (plural), and genetically modified (recombinant) bacterial cells, bacteria and bacterial strains thereof.

As used herein, the terms “commensal bacteria” and “commensal microbe” are used interchangeably herein and refer to a bacterium, bacteria (singular or plural), bacterial cell or bacterial strain that is commensal with an animal host or animal cell(s). In certain embodiments, commensal bacteria refers to a bacterium, bacteria (singular or plural), bacterial cell or bacterial strain that is commensal with a vertebrate host or vertebrate cells. In certain embodiments, commensal bacteria refers to a bacterium, bacteria (singular or plural), bacterial cell or bacterial strain that is commensal with a mammalian host or mammalian cells. In certain embodiments, commensal bacteria refers to a bacterium, bacteria (singular or plural), bacterial cell or bacterial strain that is commensal with a human host. In certain embodiments, commensal bacteria refers to a bacterium, bacteria (singular or plural), bacterial cell or bacterial strain that is commensal with human cells. In certain embodiments, the commensal bacterial act on the host's immune system. In certain embodiments and as understood by one of ordinary skill in the art, most commensal bacteria are typically symbiotic, but a commensal strain can become pathogenic or cause pathology under certain conditions, such as host immunodeficiency, microbial dysbiosis or intestinal barrier impairment. In certain embodiments, for example, a commensal bacteria is present as a non-pathogenic member of a host gut microbiome, a host skin microbiome, a host mucosal microbiome, or other host niche microbiome.

As used herein, the terms “colonization,” “colonized,” or “colonize” refers to the occupation of a microbe, e.g., a live, recombinant, commensal bacteria, in a niche of a host. In certain embodiments, colonization can be persistent, e.g. lasting over 60 days, or transient, e.g. lasting between one to 60 days.

As used herein, the terms “heterologous” or “non-native” refer to a molecule (e.g., peptide or protein) that is not normally or naturally produced or expressed by a cell or organism.

The term “antigen” refers to a molecule (e.g., peptide or protein) or immunologically active fragment thereof that is capable of eliciting an immune response. Peptide antigens are typically presented by an APC to an immune cell, such as a T lymphocyte (also called a T cell).

The terms “heterologous antigen,” or, in reference to proteins or peptides, “non-native antigen”, refer to a peptide, protein, or antigen that is not normally expressed by a cell or organism. In certain embodiments, term includes antigens, or fragments thereof, that bind to a T cell receptor and induce an immune response. In certain embodiments, for example, protein or peptide antigens are digested by APCs into short peptides that are expressed on the cell surface of an APC in the context of a major histocompatibility complex (MHC) class I or MHC-II molecule. In certain embodiments, the term antigen includes the peptides presented by an APC and recognized by a T cell receptor. In certain embodiments, heterologous antigens or non-native antigens may be host-derived antigens, or non-host derived antigens.

The term “fusion peptide” and “fusion protein” are used interchangeably herein and refer to a recombinant protein comprising two or more proteins or peptides expressed in the same amino acid chain in sequence. In certain embodiments, the two or more protein or peptide nucleic acid coding sequences can be expressed sequentially in a single open reading frame of a vector or expression plasmid. In certain embodiments, the resulting peptide or protein thus comprises a single amino acid chain with two or more proteins of interest connected via end-to-end fusion at the N- or C-termini.

In reference to microbial niches in a host, the term “native” refers to an environment in or on a host in which a commensal microorganism or host immune cell is naturally present under normal, non-pathogenic conditions.

In reference to proteins expressed by a microorganism, e.g., a bacterium, the term “native” refers to a protein, or portion thereof, that is normally expressed and present in a wild-type microorganism in nature.

The term “effective amount,” or “therapeutically effective amount,” refers to an amount of a composition sufficient to prevent, decrease or eliminate one or more symptoms of a medical condition or disease when administered to a subject in need of treatment.

As used herein, the term “operably linked” refers to a functional linkage between one or more nucleic acid sequences, such as between a regulatory or promoter sequence and a coding region sequence, where transcription of the coding region sequence is positively or negatively regulated by the linked regulatory sequence.

As used herein, “antigen-specific” refers to an immune response generated in a host that is specific to a given antigen. The term includes responses to antigens that are recognized by antibodies capable of binding to the antigen of interest with high affinity, and responses to antigens by T cell receptors (TCRs) that recognize and bind to a complex comprising an MHC molecule and a short peptide that is a degradation product of the antigen of interest. In certain embodiments, bacterial antigens are typically processed into peptides that bind to MHC-II molecules on the surface of APCs, which are recognized by the TCR of a T cell.

As used herein, “antigen-presenting cell” or “APC” refers to an immune cell that mediates a cellular immune response in a subject by processing and presenting antigens for recognition by lymphocytes such as T cells. APCs display antigen complexed with MHC on their surfaces, often referred to as “antigen presentation.” In certain embodiments, APCs can present antigen to helper T cells (CD4+ T cells) and can be referred to as professional APCs. Examples of professional APCs include dendritic cells, macrophages, Langerhans cells and B cells.

The term “regulatory T cell” or “T_{reg}” refers to a subpopulation of T cells that modulate the immune system, maintain tolerance to self-antigens, and prevent autoimmune disease. T_{regs} suppress activation, proliferation and cytokine production of CD4+ T cells and CD8+ T cells, and also suppress B cells and dendritic cells. There are two types of T_{reg} cells. “Natural” T_{regs} are produced in the thymus, whereas T_{regs} that differentiate from naïve T cells outside the thymus (in the periphery) are called “adaptive” T_{regs}. In certain embodiments, natural T_{regs} express the CD4 T cell receptor and CD25 (a component of the IL-2 receptor), and the transcription factor FOXP3. In certain embodiments, T_{regs} can also produce molecules, such as TGF-beta, IL-10 and adenosine, that suppress the immune response. In certain embodiments, adaptive T_{regs} express CD4, CD45RO, Foxp3, and CD25 (see “Human CD4+CD25hi Foxp3+ regulatory T cells are derived by rapid turnover of memory populations in vivo,” Vukmanovic-Stejic M, et al., J Clin Invest. 2006 September; 116(9):2423-33).

As used herein, the terms “T effector,” “effector T,” or “T_{eff}” refer to subpopulations of T cells that exert effector functions upon cell activation, mediated by the production of membrane and secreted proteins which modulate the immune system to elicit a pro-inflammatory immune response. In certain embodiments, T_{eff} cells include CD8+ cytotoxic T cells T_{H}1 cells, T_{H}2 cells, and T_{H}17 cells.

As used herein, the terms “engineered”, “recombinant” and “modified” are used interchangeably and refer to an organism, microorganism, cell, or bacteria that does not exist in nature. In certain embodiments, the engineered bacteria is an engineered commensal bacteria (also referred to as “engineered commensal” or “engineered commensals” herein).

As used herein, an “autoimmune disease” refers to a disease or pathological condition associated with or caused by the immune system attacking the body's endogenous organs, tissues, and/or cells.

As used herein, an “autoimmune antigen” refers to an antigen expressed by an endogenous organ, tissue or cell that triggers an immune response against the endogenous organ, tissue or cell.

As used herein, “animal” refers to an animal or an animal cell. In certain embodiments, an animal is a mammal (e.g., murines, simians, equines, bovines, porcines, canines, felines, and the like). In certain embodiments, an animal is a human. In certain embodiments, an animal is an organism to be treated or treated with a recombinant commensal microbe. In certain embodiments, the commensal microbe is an engineered bacterium or a surface-labeled bacterium.

As used herein, “host” refers to a non-microbial organism in or on which a commensal microorganism colonizes. In certain embodiments, “host” refers to a non-microbial organism in or on which a commensal bacteria colonizes. In certain embodiments, the host is an animal. In certain embodiments, the host is a mammal, In certain embodiments, the host is a human.

As used herein, the terms “subject” or “patient” are used interchangeably, and refer to any animal classified as a mammal, including humans, domestic and farm animals, and zoo, sports, or pet animals, such as dogs, horses, cats, cows, etc. In certain embodiments, the subject is a human. In certain embodiments, a subject refers to an organism to which a modified microorganism is administered. In certain embodiments, the administered modified microorganism is a live recombinant commensal bacteria of the present invention. In certain embodiments, a subject has an autoimmune or proliferative disease, disorder or condition.

As used herein, the term “pharmaceutically acceptable carrier” refers to any of the standard pharmaceutical carriers, such as phosphate buffered saline (PBS) solution, water, emulsions (e.g., such as oil/water or water/oil emulsions), and various types of wetting agents. The compositions also can include stabilizers and preservatives. For examples of carriers, stabilizers, and adjuvants, see e.g., Martin, Remington's Pharmaceutical Sciences, 15^{th} Ed. Mack Publ. Co., Easton, PA [1975].

As used herein, “pharmaceutical formulation” and “pharmaceutical composition are used interchangeably and refer to a preparation which is in such form as to permit the biological activity of an active ingredient contained therein to be effective, and which contains no additional components which are unacceptably toxic to a subject to which the formulation would be administered.

As used herein, “some embodiments”, “certain embodiments”, and “another aspect” are used interchangeably and do not have different meanings and/or scopes.

### 2. Engineered Microorganisms

Described herein is a modified microorganism engineered to express a heterologous (e.g., non-native) antigen, and methods of inducing an immune response to the heterologous (e.g., non-native) antigen in a subject. In some embodiments, the modified microorganism includes live microorganisms that colonize or are commensal in humans, such as bacteria, Archaea and fungi. In some embodiments, the live modified microorganism is a live engineered bacterium, live engineered bacteria or a live engineered bacterial strain engineered to express a heterologous antigen. In one aspect, the engineered bacteria is a commensal bacteria that expresses a non-native protein or peptide (e.g., antigen) that is capable of inducing an antigen-specific immune response in a subject. Unlike the innate and adaptive immune response to commensal bacteria, the present disclosure provides engineered bacterial strains that express a non-native protein or peptide (e.g., antigen), such as a mammalian antigen. In some embodiments, the non-native antigen is a protein or peptide that is non-native to the commensal bacterium but is native to the host. In some embodiments, the non-native antigen is a protein or peptide that is non-native to both the commensal bacterium and the host. Because the modified bacteria are derived from a bacteria that is commensal in the host, they are not expected to be pathogenic when administered to the subject.

In some embodiments, the engineered microorganism, or pharmaceutical composition comprising the engineered microorganism, is administered to a native host niche. For example, a live, recombinant commensal bacterium derived from a commensal bacterium native to a host gut niche, is administered to the same host gut niche for colonization. In another example, an engineered bacterium derived from a commensal bacterium native to a host skin niche, is administered to the same host skin niche for colonization.

In some embodiments, the engineered microorganism, e.g., the live, recombinant commensal bacterium, persistently colonizes a native host niche when administered to a subject. For example, in some embodiments, the live, recombinant commensal bacterium persists in the native host niche for over 60 days, over 112 days, over 178 days, over 1 year, over 2 years, or over 5 years. As an illustrative non-limiting example, Staphylococcus epidermidis can colonize skin of mice for at least 180 days post-association.

In some embodiments, the engineered microorganism, e.g., the live, recombinant commensal bacterium, transiently colonizes a native host niche when administered to a subject. For example, in some embodiments, the live, recombinant commensal bacterium transiently colonizes the native host niche for between 1 and 60 days, 2 and 60 days, 10 and 60 days, 20 and 60 days, 40 and 60 days, 1 and 40 days, 2 and 40 days, 10 and 40 days, 20 and 40 days, 1 and 20 days, 2 and 20 days, 10 and 20 days, 1 and 10 days, or 2 and 10 days. In some embodiments, the modified microorganism transiently colonizes the native host niche in the subject then migrates to a different niche within the host.

In some embodiments, recombinant modification of a microorganism, e.g., a live commensal bacterium, does not affect the ability of the microorganism to colonize its native host niche when administered to a subject. For example, in some embodiments, recombinant modification of a live commensal bacterium to express a non-native protein or peptide does not substantially affect the native physiology of the commensal bacterium, thereby maintaining the ability of the commensal bacterium to participate in its native synergistic interactions with the host and/or other microbial flora present in its native host niche, and facilitating the commensal bacterium's colonization of its native host niche.

The engineered bacteria described herein are useful for inducing an antigen-specific immune response to a non-native protein or peptide (e.g., a non-native antigen), which results in the generation or expansion of T cells that express a T cell receptor that specifically binds to the heterologous antigen or an immunologically active fragment thereof. Thus, the engineered bacteria can be used to treat a disease or condition in a subject by administering a therapeutically effective amount of the engineered bacteria, or a pharmaceutical composition comprising the engineered bacteria, to a subject. Following administration, the subject's immune system responds by producing antigen-specific T cells that bind the heterologous antigen expressed by the bacteria. In some embodiments, the immune system responds by producing antigen-specific regulatory T cells (T_{reg}), which reduce the host's immune response against a self-antigen or other antigen corresponding to the expressed heterologous protein or peptide. In some embodiments, the immune system responds by producing antigen-specific T cells (T_{eff}), which modulate an immune response against the expressed non-native protein or peptide, e.g., a tumor associated antigen, neoantigen, or an antigen associated with an infectious disease. In some embodiments, the immune system responds by producing antigen-specific T_{H}1 cells, which modulate an immune response against the expressed heterologous antigen, such as through promoting cellular immunity (e.g., promoting an immune environment conducive to an antigen-specific CD8 cytotoxic T cell response). In some embodiments, the immune system responds by producing antigen-specific T_{H}2 cells, which modulate an immune response against the expressed heterologous antigen, such as through promoting humoral immunity (e.g., promoting an immune environment conducive to an antigen-specific B cell response and production of antibodies). In some embodiments, the immune system responds by producing antigen-specific T helper 17 cells (T_{H}17), which modulate an immune response against the expressed heterologous antigen. In some embodiments, the immune system responds by producing antigen-specific T follicular helper cells (T_{FH}), which modulate an immune response against the expressed heterologous antigen. In some embodiments, the immune system responds by producing antigen-specific B cells, which modulate an immune response (e.g., a humoral immune response) against the expressed heterologous antigen.

In some embodiments, antigen-specific immune responses induced by engineered commensals or other engineered bacteria can be localized to the site of administration of the engineered commensals or other engineered bacteria. In some embodiments, antigen-specific immune responses induced by engineered commensals or other engineered bacteria can be restricted to the site of administration of the engineered commensals or other engineered bacteria. In some embodiments, antigen-specific immune responses induced by engineered commensals or other engineered bacteria can be distal to the site of administration of the engineered commensals or other engineered bacteria. In some embodiments, antigen-specific immune responses can include both a localized and distal immune response relative to the site of administration of the engineered commensals or other engineered bacteria.

In some embodiments, antigen-specific immune responses induced by engineered commensals or other engineered bacteria can be localized to a native host niche colonized by the engineered commensals or other engineered bacteria (e.g., a specific organ, such as skin). In some embodiments, antigen-specific immune responses induced by engineered commensals or other engineered bacteria can be restricted to a native host niche colonized by the engineered commensals or other engineered bacteria. In some embodiments, antigen-specific immune responses induced by engineered commensals or other engineered bacteria can be distal to a native host niche colonized by the engineered commensals or other engineered bacteria (e.g., an antigen-specific immune response in an organ, or site in a subject, that is not colonized by the engineered commensals or other engineered bacteria). For example, a distal antigen-specific immune response can include stimulation of immune cells at a native host niche colonized by the engineered commensals or other engineered bacteria followed by migration of the immune cells to another site (e.g., another organ). As a non-limiting illustrative example, engineered commensals or other engineered bacteria that colonize the skin can induce an antigen-specific immune response that results in immune cells (e.g., antigen-specific T cells) carrying out their effector function in organs other than the skin. In certain embodiments, the organ other than the skin is the lungs, breasts, prostate, colon, bladder, uterus, kidney, liver, pancreas, thyroid, or ovaries. In some embodiments, antigen-specific immune responses can include both a localized and distal immune response relative to a native host niche. In certain embodiments, the antigen-specific immune response targets metastases, such as skin melanoma that has metastasized to other organs.

In some embodiments, distal antigen-specific immune responses are distal relative to the site of administration of the engineered commensals or other engineered bacteria. In some embodiments, distal antigen-specific immune responses are distal relative to a host niche colonized by the engineered commensals or other engineered bacteria. In some embodiments, distal antigen-specific immune responses are in the same organ as the site of administration of the engineered commensals or other engineered bacteria and/or the native host niche colonized by the engineered commensals or other engineered bacteria. In certain embodiments, the engineered commensal or other engineered bacteria is applied to and/or colonizes one area of skin and produces an immune response in a separate part of the skin, such as a melanoma skin metastasis. In some embodiments, distal antigen-specific immune responses are in a different organ as the site of administration of the engineered commensals or other engineered bacteria and/or the native host niche colonized by the engineered commensals or other engineered bacteria. In certain embodiments, the engineered commensals or other engineered bacteria is applied to and/or colonizes the skin and produces an immune response in an organ other than skin, such as a melanoma that has metastasized to other organs. In some embodiments, distal antigen-specific immune responses are in both the same organ and a different organ as the site of administration of the engineered commensals or other engineered bacteria and/or the native host niche colonized by the engineered commensals or other engineered bacteria. In certain embodiments, the engineered commensals or other engineered bacteria is applied to and/or colonizes the skin and produces an immune response both in the skin and in an organ other than skin, such as targeting skin melanoma and targeting melanoma that has metastasized to other organs.

In certain embodiments, the modified microorganism (e.g., bacteria, Archaea, and fungi) and methods described herein provide the advantage of generating an immune response specific for a heterologous antigen when administered to a subject. In certain embodiments, the modified microorganisms described herein provide advantages over current approaches for generating antigen-specific immune cells, such as chimeric antigen receptor T cells (CAR-T cells), which are difficult and expensive to produce, are of questionable durability, and are potentially unsafe when administered to a patient because of off-target effects such as cytokine release syndrome, neurologic toxicity, and chromosomal changes caused by the CRISPR gene editing methods of eukaryotic cells. In contrast, modified microorganisms (i.e., engineered commensal microorganisms and other engineered microorganisms) are useful to trigger potent and long-lasting immune responses, and can be administered over the lifetime of a subject with no, or minimal, off-target effects. In certain embodiments, live, modified microorganisms (i.e., engineered commensal microorganisms and other engineered microorganisms) provide advantages over attenuated, pathogenic commensal and non-commensal microorganisms, e.g., attenuated Listeria, which would be undesirable to administer to subjects over long time periods. Administering attenuated, pathogenic non-commensal bacteria introduces risk to a subject, especially over a long duration, due to the potential of the attenuated bacteria to revert back to a pathogenic form. In contrast, live, commensal and non-commensal, non-pathogenic bacteria can colonize the host subject in a non-pathogenic form for potentially long time periods, and thus provide an ongoing stimulus leading to a persistent antigen-specific T cell population, which is important since T cell responses can be short-lived. In certain embodiments, recombinant S. epidermidis can persistently colonize the skin of a subject (e.g., for at least 180 days post-association) and provide an ongoing source of antigens and/or stimulus.

In some embodiments, the engineered microorganism is engulfed by an APC, such as a dendritic cell, a splenic dendritic cell, a CD8+ dendritic cell, a CD11b+ dendritic cell, a plasmacytoid dendritic cell, a follicular dendritic cell, a monocytic cell, a macrophage, a bone marrow-derived macrophage, a Kupffer cell, a B-cell, a Langerhans cell, an innate lymphoid cell, a microglia, or an intestinal epithelial cell. In certain embodiments, after being engulfed by an APC, the modified microorganism is lysed and the heterologous antigen is digested and presented to an immune cell. In some embodiments, the heterologous antigen is a protein or peptide and is processed into smaller peptide fragments, and the peptide fragments bind MHC molecules and are displayed on the surface of the APC for presentation to an immune cell. In some embodiments, the immune cell is a naïve T cell. In some embodiments, the immune cell is an antigen-experienced T cell. In some embodiments, the immune cell is a CD8+ cytotoxic T cell. The antigen-specific immune response can be elicited in vitro or in vivo. In some embodiments, the modified microorganism is engulfed, processed and presented by an APC to induce a T_{reg} response to the heterologous antigen. In some embodiments, the modified microorganism (e.g., recombinant commensal bacterium or other engineered bacteria) is engulfed, processed and presented by an APC to induce a T_{eff} response to the heterologous antigen. In some embodiments, the modified microorganism (e.g., recombinant commensal bacterium or other engineered bacteria) is engulfed, processed and presented by an APC to induce a CD8+ cytotoxic T cell response to the heterologous antigen. In some embodiments, the modified microorganism (e.g., recombinant commensal bacterium or other engineered bacteria) is engulfed, processed and presented by an APC to induce a T_{H}1 response to the heterologous antigen. In some embodiments, the modified microorganism (e.g., recombinant commensal bacterium or other engineered bacteria) is engulfed, processed and presented by an APC to induce a T_{H}2 response to the heterologous antigen.

### 3. Bacterial Surface Display System

Certain organisms, such as bacteria (e.g., commensal bacteria) including the gram-positive bacterium Firmicutesi, have potent immunomodulatory capability but have thus far been difficult to study due to the lack of existing genetic engineering tools and resistance of these bacteria to genetic manipulation. Sortase enzymes are ubiquitous among gram-positive bacteria and mediate the anchoring of proteins to bacterial cell walls. Sortase A (SrtA) is a transpeptidase expressed in Staphylococcus aureus and catalyzes the covalent linkage between a SrtA motif having the amino acid sequence LPXTG and N-terminal glycines.

Provided herein is a bacterial surface display system comprising (a) a fusion protein comprising a cell-surface tethering moiety (b) a bacterium; and (c) a protein or gene encoding the same capable of catalyzing a covalent attachment of the cell-surface tethering moiety to a cell wall of the bacterium thereby displaying the fusion protein on a bacterial surface. In some embodiments, the cell wall tethering moiety comprises a SrtA motif and the protein capable of catalyzing the covalent attachment is a SrtA protein. For example, in some embodiments, SrtA catalyzes the covalent linkage of the fusion protein to surface proteins expressing N-terminal glycine residues on the outer surface of the commensal bacterium.

In some embodiments, the bacterium is a commensal bacterium. In some embodiments, the bacterium is a gram positive commensal bacterium and SrtA catalyzes the covalent linkage of the fusion protein to a cell wall protein expressing N-terminal glycine residues. In other embodiments, the bacterium is a gram negative bacterium and SrtA catalyzes the covalent linkage of the fusion protein to an outer membrane protein expressing N-terminal glycine residues.

In some embodiments, the cell wall or outer membrane protein comprises 2 to 20 N-terminal glycine residues. For example, the cell wall or outer membrane fusion protein comprises 2, 3, 4, 5, 6, 7, 8, 9, 10, 11, 12, 13, 14, 15, 16, 17, 18, 19 or 20 N-terminal glycine residues.

In some embodiments, the fusion protein comprises a protein or peptide that is non-native to the bacterium. For example, in some embodiments, the non-native protein or peptide comprises a non-native antigenic protein or peptide. In some embodiments the protein or peptide is associated with a host disease or condition, for example, an infection, a proliferative disorder, or an autoimmune disorder. In certain embodiments, the protein or peptide elicits a host adaptive immune response, e.g., a T cell response.

In some embodiments, the fusion protein comprises a non-native protein or peptide that facilitates molecular labeling or targeting to specialized cells. For example, in some embodiments, the fusion protein comprises a nanobody (VHH) against GFP comprising the sequence of SEQ ID NO:61. In other embodiments, for example, the fusion protein comprises a VHH domain targeting APCs (e.g., anti-CD11b VHH comprising the sequence of SEQ ID NO:34, or anti-MHC-II VHH comprising the sequence of SEQ ID NO:33.

In some embodiments, the fusion protein is recombinantly expressed in vitro and contacted with the bacterium in the presence or SrtA. In other embodiments, the fusion protein is recombinantly expressed and secreted by a second bacterium. In some embodiments, SrtA is recombinantly expressed in vitro and contacted with the bacterium in the presence of the fusion protein. In other embodiments, SrtA is recombinantly expressed and secreted by a second bacterium. In certain embodiments, the fusion protein is expressed and secreted and the SrtA is expressed by the same bacterium. In certain embodiments, the fusion protein is expressed and secreted and the SrtA is expressed by the same second bacterium and catalyze the linkage of the fusion protein to the surface of a first bacterium.

In some embodiments, the bacterium with the surface displayed fusion protein (also referred to as a “surface-labeled bacterium” herein) includes live microorganisms that colonize or are commensal in humans, such as bacteria, archaea and fungi. In some embodiments, the surface-labeled bacterium is a live engineered bacterium, or a live engineered bacterium displaying a heterologous antigen. In some embodiments, the live surface-labeled bacterium is a live engineered bacterium, or a live engineered bacterial strain engineered to express a heterologous antigen. In one aspect, the engineered bacteria is a commensal bacteria that expresses a non-native protein or peptide (e.g., antigen) that is capable of inducing an antigen-specific immune response in a subject. Unlike the innate and adaptive immune response to commensal bacteria, the present disclosure provides surface-labeled bacteria that can display a non-native protein or peptide (e.g., antigen) or surface-labeled bacteria that can be engineered to express a non-native protein or peptide (e.g., antigen), such as a mammalian antigen. In some embodiments, the non-native antigen is a protein or peptide that is non-native to the surface-labeled bacteria, such as a surface-labeled commensal bacterium, but is native to the host. In some embodiments, the non-native antigen is a protein or peptide that is non-native to both the commensal bacterium and the host. Because the surface-labeled bacteria can be derived from a bacteria that is commensal in the host, they are not expected to be pathogenic when administered to the subject.

In some embodiments, the surface-labeled bacteria, or a pharmaceutical composition comprising the surface-labeled bacteria, is administered to a native host niche. For example, a live, recombinant commensal bacterium derived from a commensal bacterium native to a host gut niche, is administered to the same host gut niche for colonization. In another example, a surface-labeled bacterium derived from a commensal bacterium native to a host skin niche, is administered to the same host skin niche for colonization.

In some embodiments, the surface-labeled bacteria, e.g., the live, recombinant commensal bacterium, persistently colonizes a native host niche when administered to a subject. For example, in some embodiments, the live, recombinant commensal bacterium persists in the native host niche for over 60 days, over 112 days, over 178 days, over 1 year, over 2 years, or over 5 years.

In some embodiments, the surface-labeled bacteria, e.g., the live, recombinant commensal bacterium, transiently colonizes a native host niche when administered to a subject. For example, in some embodiments, the live, recombinant commensal bacterium transiently colonizes the native host niche for between 1 and 60 days, 2 and 60 days, 10 and 60 days, 20 and 60 days, 40 and 60 days, 1 and 40 days, 2 and 40 days, 10 and 40 days, 20 and 40 days, 1 and 20 days, 2 and 20 days, 10 and 20 days, 1 and 10 days, or 2 and 10 days. In some embodiments, the surface-labeled bacteria transiently colonizes the native host niche in the subject then migrates to a different niche within the host.

In some embodiments, recombinant modification of a microorganism, e.g., a live commensal bacterium, does not affect the ability of the microorganism to colonize its native host niche when administered to a subject. For example, in some embodiments, recombinant modification of a live commensal bacterium to express a non-native protein or peptide does not substantially affect the native physiology of the commensal bacterium, thereby maintaining the ability of the commensal bacterium to participate in its native synergistic interactions with the host and/or other microbial flora present in its native host niche, and facilitating the commensal bacterium's colonization of its native host niche.

In certain embodiments, the surface-labeled bacteria described herein are useful for inducing an antigen-specific immune response to a non-native protein or peptide (e.g., a non-native antigen), which results in the generation or expansion of T cells that express a T cell receptor that specifically binds to the heterologous antigen or an immunologically active fragment thereof. Thus, the surface-labeled bacteria can be used to treat a disease or condition in a subject by administering a therapeutically effective amount of the surface-labeled bacteria, or a pharmaceutical composition comprising the surface-labeled bacteria, to a subject. Following administration, the subject's immune system responds by producing antigen-specific T cells that bind the heterologous antigen expressed by the bacteria. In some embodiments, the immune system responds by producing antigen-specific regulatory T cells (T_{reg}), which reduce the host's immune response against a self-antigen or other antigen corresponding to the expressed heterologous protein or peptide. In some embodiments, the immune system responds by producing antigen-specific T effector cells (T_{eff}), which modulate an immune response against the expressed non-native protein or peptide, e.g., a tumor associated antigen, neoantigen, or an antigen associated with an infectious disease. In some embodiments, the immune system responds by producing antigen-specific T_{H}1 cells, which modulate an immune response against the expressed heterologous antigen, such as through promoting cellular immunity (e.g., promoting an immune environment conducive to an antigen-specific CD8+ cytotoxic T cell response). In some embodiments, the immune system responds by producing antigen-specific T_{H}2 cells, which modulate an immune response against the expressed heterologous antigen, such as through promoting humoral immunity (e.g., promoting an immune environment conducive to an antigen-specific B cell response and production of antibodies). In some embodiments, the immune system responds by producing antigen-specific T helper 17 cells (T_{H}17), which modulate an immune response against the expressed heterologous antigen. In some embodiments, the immune system responds by producing antigen-specific T follicular helper cells (T_{FH}), which modulate an immune response against the expressed heterologous antigen. In some embodiments, the immune system responds by producing antigen-specific B cells, which modulate an immune response (e.g., a humoral immune response) against the expressed heterologous antigen.

In certain embodiments, the surface-labeled bacterium and methods described herein provide the advantage of generating an immune response specific for a heterologous antigen when administered to a subject. The disclosure also provides advantages over current approaches for generating antigen-specific immune cells, such as chimeric antigen receptor T cells (CAR-T cells), which are difficult and expensive to produce, are of questionable durability, and are potentially unsafe when administered to a patient because of off-target effects such as cytokine release syndrome and neurologic toxicity. In contrast, commensal microorganisms can be useful to trigger potent and long-lasting immune responses, and can be administered over the lifetime of a subject with no, or minimal, off-target effects. Live, commensal microorganisms thus provide advantages over attenuated, pathogenic non-commensal microorganisms, e.g., attenuated Listeria, which would be undesirable to administer to subjects over long time periods. Administering attenuated, pathogenic non-commensal bacteria introduces risk to a subject, especially over a long duration, due to the potential of the attenuated bacteria to revert back to a pathogenic form. In contrast, live, commensal bacteria can colonize the host subject in a non-pathogenic form for potentially long time periods, and thus provide an ongoing stimulus leading to a persistent antigen-specific T cell population, which is important since T cell responses can be short-lived.

In some embodiments, the surface-labeled bacteria is engulfed by an APC, such as a dendritic cell, a splenic dendritic cell, a CD8+ dendritic cell, a CD11b+ dendritic cell, a plasmacytoid dendritic cell, a follicular dendritic cell, a monocytic cell, a macrophage, a bone marrow-derived macrophage, a Kupffer cell, a B-cell, a Langerhans cell, an innate lymphoid cell, a microglia, or an intestinal epithelial cell. After being engulfed by an APC, the surface-labeled bacterium is lysed and the heterologous antigen is digested and presented to an immune cell. In some embodiments, the heterologous antigen is a protein or peptide and is processed into smaller peptide fragments, and the peptide fragments bind MHC molecules (e.g., MHC-I or MHC-II) and are displayed on the surface of the APC for presentation to an immune cell. In some embodiments, the immune cell is a naïve T cell. In some embodiments, the immune cell is an antigen-experienced T cell. In some embodiments, the immune cell is a CD8+ cytotoxic T cell. The antigen-specific immune response can be elicited in vitro or in vivo. In some embodiments, the surface-labeled bacterium is engulfed, processed and presented by an APC to induce a T_{reg} response to the heterologous antigen. In some embodiments, the surface-labeled bacterium (e.g., recombinant commensal bacterium) is engulfed, processed and presented by an APC to induce a T_{eff} response to the heterologous antigen. In some embodiments, the surface-labeled bacterium (e.g., recombinant commensal bacterium) is engulfed, processed and presented by an APC to induce a CD8+ cytotoxic T cell response to the heterologous antigen. In some embodiments, the surface-labeled bacterium (e.g., recombinant commensal bacterium) is engulfed, processed and presented by an APC to induce a T_{H}1 response to the heterologous antigen. In some embodiments, the surface-labeled bacterium (e.g., recombinant commensal bacterium) is engulfed, processed and presented by an APC to induce a T_{H}2 response to the heterologous antigen.

### 4. Bacterial Strains

In some embodiments, the modified microorganism is a live, recombinant bacteria or bacterial strain. In some embodiments, the live, recombinant bacteria is derived from a commensal bacteria or bacterial strain. In some embodiments, the live, recombinant bacteria is derived from a commensal bacteria or bacterial strain in a mammal. In some embodiments, the live, recombinant bacteria or bacterial strain is derived from a commensal bacteria or bacterial strain in a human. In some embodiments, the live, recombinant bacteria or bacterial strain is derived from a commensal bacteria or bacterial strain native in a human niche, for example, a gastrointestinal tract, respiratory tract, urogenital tract, and/or skin.

In some embodiments, the live, recombinant bacteria is derived from a commensal bacteria that is native to the digestive tract of a mammal. The live, recombinant bacterium can be a gram-negative bacteria or a gram-positive bacteria. In some embodiments, the live, recombinant bacterium is derived from a Bacteroides spp., Clostridium spp., Faecalibacterium spp., Helicobacter spp., Parabacteroides spp., or Prevotella spp. In some embodiments, the live, recombinant bacterium is derived from Bacteroides thetaiotaomicron, Bacteroides vulgatus, Bacteroides finegoldii, or Helicobacter hepaticus.

In some embodiments, the live, recombinant bacteria is derived from a commensal bacteria that is native to the skin of a mammal. For example, in some embodiments, the live, recombinant bacterium is derived from a Staphylococcus spp., or Corynebacterium spp. In some embodiments, the live, recombinant bacterium is derived from Staphylococcus epidermidis. For example, in some embodiments, the live, recombinant bacterium is derived from S. epidermidis NIHLM087.

### Gram Negative Bacteria

In some embodiments, the live, recombinant bacteria is derived from a commensal bacteria or other bacteria that is Gram negative. For example, in some embodiments, the Gram negative bacteria is a Bacteroides spp., a Helicobacter spp., or a Parabacteroides spp. In some embodiments, the live, recombinant bacterium is B. thetaiotaomicron, B. vulgatus, B. finegoldii, or H. hepaticus.

### Gram Positive Bacteria

In some embodiments, the live, recombinant bacteria is derived from a commensal bacteria or other bacteria that is Gram positive. For example, in some embodiments, the Gram positive bacteria is a Staphylococcus spp., a Faecalibacterium spp., or a Clostridium spp. In some embodiments, the live, recombinant bacterium is S. epidermidis.

In some embodiments, the live, recombinant bacteria is derived from a commensal bacteria that is known to induce a T_{reg} response in a mammalian host. In some embodiments, the live, recombinant bacteria is derived from a Bacteroides spp., Helicobacter spp., Parabacteroides spp., Clostridium spp., Staphylococcus spp., Lactobacillus spp., Fusobacterium spp., Enterococcus spp., Acenitobacter spp., Flavinofractor spp., Lachnospiraceae spp., Erysipelotrichaceae spp., Anaerostipes spp., Anaerotruncus spp., Coprococcus spp., Clostridiales spp., Odoribacter spp., Collinsella spp., Bifidobacterium spp., or Streptococcus or Prevotella spp.

In some embodiments, the live, recombinant bacterium is derived from Clostridium ramosum, Staphylococcus saprophyticus, Bacteroides thetaiotaomicron, Clostridium histolyticum, Lactobacillus rhamnosus, Parabacteroides johnsonii, Fusobacterium nucleatum, Enterococcus faecium, Lactobacillus casei, Acenitobacter lwofii, Bacteroides ovatus, Bacteroides vulgatus, Bacteroides uniformis, Bacteroides finegoldii, Clostridium spiroforme, Flavonifractor plautii, Clostridium hathewayi, Lachnospiraceae bacterium, Clostridium bolteae, Erysipelotrichaceae bacterium, Anaerostipes caccae, Anaerotruncus colihominis, Coprococcus comes, Clostridium asparagiforme, Clostridium symbiosum, Clostridium ramosum, Clostridium sp. D5, Clostridium scindens, Lachnospiraceae bacterium, Clostridiales bacterium, Bacteroides intestinalis, Bacteroides caccae, Bacteroides massiliensis, Parabacteroides distasonis, Odoribacter splanchnicus, Collinsella aerofaciens, Acinetobacter lwoffii, Bifpdobacterium breve, Bacteroides finegoldii, Bacteroides fragilis, Bacteroides massiliensis, Bacteroides ovatus, Bifidobacterium bifidum, Lactobacillus acidofilus, Lactobacillus casei, Lactobacillus reuteri, Streptococcus thermophilus, or Prevotella histicola.

In some embodiments, the live, recombinant bacterium is derived from Corynebacterium tuberculostearicum, Corynebacterium accolens, Corynebacterium accolens, Corynebacterium amycolatum, Corynebacterium aurimucosum, Corynebacterium aurimucosum, Corynebacterium propinquum, Corynebacterium pseudodiphtheriticum, Corynebacterium granulosum, Cutibacterium acnes, Cutibacterium acnes, Cutibacterium avidum, Cutibacterium avidum, Dolosigranulum pigrum, Finegoldia magna, Moraxella catarrhalis, Moraxella nonliquefaciens, Haemophilus influenzae, Haemophilus aegyptius, Rothia mucilaginosa, Streptococcus pyogenes, Streptococcus pyogenes, Streptococcus agalactiae, Streptococcus agalactiae, Streptococcus gordonii, Neisseria lactamica, Neisseria inereal, Neisseria mucosa, Lactobacillus crispatus, Lactobacillus jensenii gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium breve, Bifidobacterium longum, Veillonella parvula, Veillona parvula, Gardnerella vaginalis, Atopobium vaginae, Prevotella bivia, Mobiluncus mulieris, Mageeibacillus indolicus, Prevotella buccalis, Enterococcus faecium, Lactococcus lactis, Ruminococcus gnavus, or Eubacterium limosum. In some embodiments, the commensal bacterium is derived from a bacterium having ATCC accession number 35692, 49725, 49726, 49368, 700975, 700540, 51488, 10700, 25564, 51277, 11827, 25577, 49753, 51524, 29328, 25238, 25240, 19976, 51907, 11116, 25296, 19615, 12344, BAA-611, 13813, 10558, 23970, 14685, 19696, 33820, 25258, 19992, 55195, 4356, 33200, 7469, 393, 7995D-5, 23272, 11741, 15700, 15697, 10790, 17745, 14018, BAA-55, 29303, 35243, BAA-2120, 35310, 19434, 19435, 29149, or 8486. Commensal bacterium useful for the present invention are shown in Table 1.

TABLE 1

| Genus | Species | ATCC Accession No |
|---|---|---|
| Corynebacterium | Corynebacterium | 35692 |
| | tuberculostearicum | |
| | Corynebacterium accolens | 49725 |
| | Corynebacterium accolens | 49726 |
| | Corynebacterium amycolatum | 49368 |
| | Corynebacterium aurimucosum | 700975 |
| | Corynebacterium aurimucosum | 700540 |
| | Corynebacterium propinquum | 51488 |
| | Corynebacterium | 10700 |
| | pseudodiphtheriticum | |
| | Corynebacterium granulosum | 25564 |
| Cutibacterium/ | Cutibacterium acnes | 51277 |
| Propionibacterium | Cutibacterium acnes | 11827 |
| | Cutibacterium avidum | 25577 |
| | Cutibacterium avidum | 49753 |
| Dolosigranulum | Dolosigranulum pigrum | 51524 |
| Finegoldia | Finegoldia magna | 29328 |
| Moraxella | Moraxella catarrhalis | 25238 |
| | Moraxella catarrhalis | 25240 |
| | Moraxella nonliquefaciens | 19976 |
| Haemophilus | Haemophilus influenzae | 51907 |
| | Haemophilus aegyptius | 11116 |
| Rothia | Rothia mucilaginosa | 25296 |
| Streptococcus | Streptococcus pyogenes | 19615 |
| | Streptococcus pyogenes | 12344 |
| | Streptococcus agalactiae | BAA-611 |
| | Streptococcus agalactiae | 13813 |
| | Streptococcus gordonii | 10558 |
| Neisseria | Neisseria lactamica | 23970 |
| | Neisseria cinerea | 14685 |
| | Neisseria mucosa: | 19696 |
| Lactobacillus | Lactobacillus crispatus | 33820 |
| | Lactobacillus jensenii gasser | 25258 |
| | Lactobacillus gasseri | 19992 |
| | Lactobacillus iners | 55195 |
| | Lactobacillus acidophilus | 4356 |
| | Lactobacillus johnsonii | 33200 |
| | Lactobacillus rhamnosus | 7469 |
| | Lactobacillus casei | 393 |
| | Lactobacillus helveticus | 7995D-5 |
| | Lactobacillus reuteri | 23272 |
| | Lactobacillus salivarius | 11741 |
| Bifidobacteria | Bifidobacterium breve | 15700 |
| | Bifidobacterium longum | 15697 |
| Veillonella | Veillonella parvula | 10790 |
| | Veillonella parvula | 17745 |
| Others | Gardnerella vaginalis | 14018 |
| | Atopobium vaginae | BAA-55 |
| | Prevotella bivia | 29303 |
| | Mobiluncus mulieris | 35243 |
| | Mageeibacillus indolicus | BAA-2120 |
| | Prevotella buccalis | 35310 |
| | Enterococcus faecium | 19434 |
| | Lactococcus lactis | 19435 |
| | Ruminococcus gnavus | 29149 |
| | Eubacterium limosum | 8486 |

In some embodiments, the live, recombinant bacteria is derived from a commensal bacteria or other bacteria that is known to induce a T_{eff} response in a mammalian host. In some embodiments, the live, recombinant bacteria is derived from a Staphylococcus spp., Parabacteroides spp., Alistipes spp., Bacteroides spp., Eubacterium spp., Runimococcaceae spp., Phascolarctobacterium spp., Fusobacterium spp., Kebsiella spp., Clostridium spp., Coprobacillus spp., Erysipelotrichaceae spp., Subdoligranulum spp., Ruminococcus spp., Firmicutes spp., or Bifidobacterium spp.

In some embodiments, the live, recombinant bacteria is derived from S. epidermidis, Parabacteroides distasonis, Parabacteroides gordonii, Alistipes senegalensis, Parabacteroides johnsonii, Paraprevotella xylaniphila, Bacteroides dorei, Bacteroides unormis JCM5828, Eubacterium limosum, Ruminococcaceae bacterium cv2, Phascolarctobacterium faecium, Fusobacterium ulcerans, Klebsiella pneumoniae, Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus AGR2154, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Subdoligranulum sp. 4_3_54A2FAA, Ruminococcus bromii L2-63, Firmicutes bacterium ASF500, Bacteroides dorei 5_1_36D4 supercont2.3, Bifidobacterium animalis subsp. lactis ATCC 27673, or Bifidobacterium breve UCC2003.

Additional commensal and non-commensal bacterial strains that can be engineered to express or display non-native proteins or peptides are listed in Table 2.

TABLE 2
ADDITIONAL COMMENSAL AND NON-
COMMENSAL BACTERIAL STRAINS FOR
ENGINEERING OR USE IN A SURFACE DISPLAY SYSTEM

| | | |
|---|---|---|
| Bacteroides | Clostridium scindens | Bacteroides dorei |
| thetaiotaomicron | | |
| Bacteroides | Lachnospiraceae | Bacteroides uniformis |
| finegoldii | bacterium | JCM 5828 |
| Bacteroides vulgatus | Clostridiales bacterium | Eubacterium limosum |
| Helicobacter | Bacteroides | Ruminococcaceae |
| hepaticus | intestinalis | bacterium cv2 |
| Clostridium | Bacteroides caccae | Phascolarctobacterium |
| ramosum | | faecium |
| Staphylococcus | Bacteroides | Fusobacterium ulcerans |
| saprophyticus | massiliensis | |
| Clostridium | Parabacteroides | Klebsiella pneumoniae |
| histolyticum | distasonis | |
| Lactobacillus | Odoribacter | Clostridium bolteae |
| rhamnosus | splanchnicus | 90B3 |
| Parabacteroides | Collinsella aerofaciens | Clostridium cf. |
| johnsonii | | saccharolyticum K10 |
| Fusobacterium | Acinetobacter lwoffii | Clostridium symbiosum |
| nucleatum | | WAL-14673 |
| Enterococcus | Bifidobacterium breve | Clostridium hathewayi |
| faecium | | 12489931 |
| Lactobacillus casei | Bacteroides fragilis | Ruminococcus obeum |
| | | A2-162 |
| Acenitobacter lwofii | Bacteroides | Ruminococcus gnavus |
| | massiliensis | AGR2154 |
| Bacteroides ovatus | Bacteroides ovatus | Butyrate-producing |
| | | bacterium SSC/2 |
| Bacteroides | Bifidobacterium | Clostridium sp. ASF356 |
| uniformis | bifidum | |
| Clostridium | Lactobacillus | Coprobacillus sp. D6 |
| spiroforme | acidofilus | cont1.1 |
| Flavonifractor | Lactobacillus casei | Eubacterium sp. 3_1_31 |
| plautii | | cont1.1 |
| Clostridium | Lactobacillus reuteri | Erysipelotrichaceae |
| hathewayi | | bacterium 21_3 |
| Lachnospiraceae | Streptococcus | Subdoligranulum sp. |
| bacterium | thermophilus | 4_3_54A2FAA |
| Clostridium bolteae | Prevotella histicola | Ruminococcus bromii |
| | | L2-63 |
| Erysipelotrichaceae | Staphylococcus | Firmicutes bacterium |
| bacterium | epidermidis LM097 | ASF500 |
| Anaerostipes caccae | Corynebacterium spp. | Firmicutes bacterium |
| | | ASF500 |
| Anaerotruncus | Parabacteroides | Bacteroides dorei |
| colihominis | distasonis | 5 1 36/D4 supercont2.3 |
| Coprococcus comes | Parabacteroides | Bifidobacterium animalis |
| | gordonii | subsp. lactis ATCC 27673 |
| Clostridium | Alistipes senegalensis | Bifidobacterium breve |
| asparagiforme | | UCC2003 |
| Clostridium | Parabacteroides | Bacteroides dorei |
| symbiosum | johnsonii | |
| Clostridium | Paraprevotella | Bacteroides uniformis |
| ramosum | xylaniphila | JCM 5828 |
| Clostridium sp. D5 | Clostridium scindens | Eubacterium limosum |

### 5. Non-Native Proteins and Peptides

In some embodiments, modified microorganisms, e.g., live, recombinant commensal bacteria, are engineered to express or display a non-native protein or peptide (e.g., a heterologous antigen) that is not naturally expressed in the microorganism. For example, in some embodiments, the non-native protein or peptide normally exists in, is present in, or is expressed by a non-bacterial host. In some embodiments, the non-bacterial host is an animal that is a natural host of the commensal bacteria from which the modified microorganism is derived. In some embodiments, the non-native protein or peptide normally exists in, is present in or is expressed by the host of the commensal bacteria. In some embodiments, the non-native protein or peptide is an antigen that exists in a vertebrate or mammal. In some embodiments, the non-native protein or peptide is a mammalian antigen, such as a mouse or human antigen.

In some embodiments, the non-native protein or peptide is a protein or antigenic fragment thereof. The size of at least one antigenic peptide can be, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino acid residues, and any range derivable therein. In specific embodiments the antigenic peptide molecules are equal to or less than 50 amino acids.

In some embodiments, a non-native protein or peptide comprises one or more T cell epitopes capable of presentation by MHC-I (e.g., a non-native protein or peptide engineered to elicit a CD8+ cytotoxic T cell response) and are typically 15 residues or less in length and usually consist of between about 8 and about 11 residues, particularly 9 or 10 residues. In some embodiments, the non-native protein or peptide comprises one or more epitopes capable of presentation by MHC-II (e.g., a non-native protein or peptide engineered to elicit a CD4+ T cell response) and are typically 6-30 residues, inclusive. In some embodiments, the non-native protein or peptide is capable of undergoing antigen processing into one or more T cell epitopes capable of presentation by MHC-I and/or MHC-II. In some embodiments, the non-native protein or peptide comprises an epitope, or antigen capable of antigen processing, capable of being presented on one or more distinct HLA alleles, such as any one of HLA-A, HLA-B, HLA-C, HLA-DQ, HLA-DR, and HLA-DP.

In some embodiments, an engineered microorganism is engineered to express, or a surface-labeled bacterium displays, a single non-native protein or peptide comprising one or more T cell epitopes capable of presentation by an MHC molecule and one or more B cell epitopes capable of eliciting an antibody response. T cell epitopes and B cell epitopes can be derived from the same antigen protein. T cell epitopes and B cell epitopes can be derived from distinct antigenic proteins.

In some embodiments, an engineered microorganism is engineered to express, or a surface-labeled bacterium displays, a single non-native protein or peptide comprising two or more T cell epitopes capable of presentation by an MHC molecule. For example, a single non-native protein or peptide can comprise a T cell epitope capable of presentation by MHC-I and a T cell epitope capable of presentation by MHC-II. In some embodiments, a T cell epitope capable of presentation by MHC-I and a T cell epitope capable of presentation by MHC-II are each derived from the same antigenic protein, such as a single contiguous amino acid sequence derived from a naturally occurring antigen (e.g., a full-length protein or protein domain) or a non-natural peptide fusion (e.g. concatemer) of epitope-encoding amino acid sequences. In some embodiments, a T cell epitope capable of presentation by MHC-I and a T cell epitope capable of presentation by MHC-II are each derived from distinct antigenic proteins, such as a non-natural peptide fusion (e.g. concatemer) of epitope-encoding amino acid sequences from a first protein and epitope-encoding amino acid sequences from a second protein. In certain embodiments, the T cell epitope capable of presentation by MHC-I and a T cell epitope capable of presentation by MHC-II are encoded by a single non-native protein or peptide

In some embodiments, an engineered microorganism is engineered to express, or a surface-labeled bacterium displays, two or more non-native proteins or peptides. In some embodiments, an engineered microorganism is engineered to express two or more non-native proteins or peptides and each of the two or more non-native proteins independently comprise a T cell epitope capable of presentation by MHC-I, a T cell epitope capable of presentation by MHC-II, a B cell epitope, or combinations thereof.

In some embodiments, an engineered microorganism is engineered to express, or a surface-labeled bacterium displays, two or more non-native proteins or peptides including at least a first non-native protein or peptide that comprises one or more T cell epitopes capable of presentation by an MHC molecule and at least a second non-native protein or peptide that comprises one or more B cell epitopes capable of eliciting an antibody response. T cell epitopes and B cell epitopes can be derived from the same antigenic protein. T cell epitopes and B cell epitopes can be derived from distinct antigenic proteins.

In some embodiments, an engineered microorganism is engineered to express, or a surface-labeled bacterium displays, two or more non-native proteins or peptides including at least a first non-native protein or peptide that comprises one or more T cell epitopes capable of presentation by MHC-I and at least a second non-native protein or peptide that comprises one or more T cell epitopes capable of presentation by MHC-II. MHC-I T cell epitopes and MHC-II T cell epitopes can be derived from the same antigenic protein. MHC-I T cell epitopes and MHC-II T cell epitopes can be derived from distinct antigenic proteins.

In some embodiments, two or more engineered microorganisms can be engineered to express, or two or more surface-labeled bacteria display, one or more non-native proteins or peptides.

In some embodiments, two or more engineered microorganisms can be engineered to express, or two or more surface-labeled bacteria display, one or more non-native proteins or peptides including at least a first engineered microorganism engineered to express, or a first surface-labeled bacterium displays, a first non-native protein or peptide that comprises one or more T cell epitopes capable of presentation by an MHC molecule and at least a second engineered microorganism engineered to express, or a second surface-labeled bacterium displays, a second non-native protein or peptide that comprises one or more B cell epitopes capable of eliciting an antibody response. In certain embodiments, T cell epitopes and B cell epitopes expressed by distinct engineered microorganisms or surface-labeled bacteria, can be derived from the same antigenic protein. In certain embodiments, T cell epitopes and B cell epitopes expressed by distinct engineered microorganisms or surface-labeled bacteria can be derived from distinct antigenic proteins.

In some embodiments, two or more engineered microorganisms can be engineered to express, or two or more surface-labeled bacteria display, one or more non-native proteins or peptides including at least a first engineered microorganism engineered to express a first non-native protein or peptide comprising one or more T cell epitopes capable of presentation by MHC-I and at least a second engineered microorganism engineered to express a second non-native protein or peptide comprising one or more T cell epitopes capable of presentation by MHC-II. In certain embodiments, MHC-I T cell epitopes and MHC-II T cell epitopes expressed by distinct engineered microorganisms, or surface-labeled bacteria, can be derived from the same antigenic protein. In certain embodiments, MHC-I T cell epitopes and MHC-II T cell epitopes expressed by distinct engineered microorganisms, or surface-labeled bacteria, can be derived from distinct antigenic proteins.

In some embodiments, the modified microorganism is capable of inducing a regulatory T cell response in the host to the non-native protein or peptide the modified microorganism is engineered to express, or the surface-labeled bacterium displays. In some embodiments, the modified microorganism is a live, recombinant commensal bacteria that is capable of inducing a regulatory T cell response in the host to the non-native protein or peptide the modified microorganism is engineered to express, or the surface-labeled bacterium displays. In certain embodiments, when the non-native protein or peptide or heterologous antigen is presented on the surface of an antigen presenting cell to a naïve T cell, the naïve T cell will differentiate into a T_{reg} cell. As is known in the art, differentiation into a T_{reg} cell can be induced under appropriate conditions, such as the presence of cytokines including TGF-β. Without intending to be bound by a particular mechanism, the modified microorganism may induce production of cytokines by an APC that favor the differentiation of naïve T cells to T_{reg} cells. In certain embodiments, the modified microorganism is a live, recombinant commensal bacteria that may induce production of cytokines by an APC that favor the differentiation of naïve T cells to T_{reg} cells. In some embodiments, the modified microorganism induces a T_{reg} response to the heterologous antigen, but does not elicit an immune response mediated by other subsets of T cells, such as CD8+ or T_{H}17 T cells. In some embodiments, the modified microorganism is a live, recombinant commensal bacteria that induces a T_{reg} response to the heterologous antigen, but does not elicit an immune response mediated by other subsets of T cells, such as CD8+ or T_{H}17 T cells. In some embodiments, the modified microorganism induces a T_{H}2 response to the heterologous antigen. In some embodiments, the modified microorganism is a live, recombinant commensal bacteria that induces a T_{H}2 response to the heterologous antigen.

In some embodiments, the modified microorganisms express the heterologous antigen at a level that is sufficient to trigger an immune response when the microorganism is engulfed by an APC and the antigen, or antigenic fragment thereof, is presented to a T cell in the context of an HLA molecule. In some embodiments, the modified microorganisms is a live, recombinant commensal bacteria that can express the heterologous antigen at a level that is sufficient to trigger an immune response when the microorganism is engulfed by an APC and the antigen, or antigenic fragment thereof, is presented to a T cell in the context of an HLA molecule. Methods for optimizing protein expression levels in bacteria are described in Rosano G., et al. “Recombinant protein expression in Escherichia coli: advances and challenges,” Front Microbiol. 2014; 5: 172 (Published online 2014 Apr. 17).

In some embodiments, the non-native protein or peptide or heterologous antigen comprises non-natural amino acids. A “non-natural amino acid” refers to an amino acid that is not one of the 20 common amino acids and includes, but is not limited to, amino acids which occur naturally by modification of a naturally encoded amino acid (including but not limited to, the 20 common amino acids) but are not themselves incorporated into a growing polypeptide chain by the translation complex. Non-limiting examples of naturally-occurring amino acids that are not naturally-encoded include, but are not limited to, N-acetylglucosaminyl-L-serine, N-acetylglucosaminyl-L-threonine, and O-phosphotyrosine. Additionally, the term “non-natural amino acid” includes, but is not limited to, amino acids which do not occur naturally and may be obtained synthetically or may be obtained by modification of non-natural amino acids.

In certain embodiments, expression of the non-native protein or peptide or heterologous antigen by the modified microorganisms can be detected using assays that detect expression of the antigen RNA or protein, such as RT-PCR, Northern analysis, microarray, or Western blot. In certain embodiments, expression of the non-native protein or peptide or heterologous antigen by modified microorganisms that are live, recombinant commensal bacteria can be detected using assays that detect expression of the antigen RNA or protein, such as RT-PCR, Northern analysis, microarray, or Western blot.

In some embodiments, a non-native protein or peptide or heterologous antigen described herein is linked to an endogenous protein, or functional fragment of an endogenous protein, expressed by a commensal bacteria or bacterial strain. In some embodiments, a non-native protein or peptide, or heterologous antigen or antigenic fragment thereof, can be linked to an endogenous commensal bacterial protein, or functional fragment thereof, to form a fusion protein that is expressed by the live, recombinant commensal bacteria. In some embodiments, the non-native protein or peptide, or heterologous antigen or antigenic fragment thereof, is fused to the N-terminus of the endogenous commensal bacterial protein, or functional fragment thereof. In some embodiments, the non-native protein or peptide, or heterologous antigen or antigenic fragment thereof, is fused to the C-terminus of the endogenous commensal bacterial protein, or functional fragment thereof. In some embodiments, the non-native protein or peptide, or heterologous antigen or antigenic fragment thereof, can be linked to the endogenous commensal bacterial protein, or functional portion thereof, by an amino acid linker. In some embodiments, the amino acid linker comprises the sequence GG.

In some embodiments, the heterologous antigen, or antigenic fragment thereof, is linked to sialidase, endonuclease, secreted endoglycosidase, anti-sigma factor, thiol peroxidase, hypothetical protein BT 2621, hypothetical protein BT_3223, peptidase, Icc family phosphohydrolase, exo-poly-alpha-D-galacturonosidase, hypothetical protein BT_4428, or functional fragments thereof.

### Autoimmune Antigens

In some embodiments, the non-native protein or peptide is an autoimmune antigen. In some embodiments, the non-native protein or peptide is myelin oligodendrocyte glycoprotein, insulin, chromogranin A, hybrid insulin peptides, proteolipid protein, myelin basic protein, villin, epithelial cellular adhesion molecule, collagen alpha-1, aggrecan core protein, 60 kDa chaperonin 2, vimentin, alpha-enolase, fibrinogen alpha chain, fibrinogen beta chain, chitinase-3-like protein, 60 kDa mitochondrial heat shock protein, matrix metalloproteinase-16, thyroid peroxidase, thyrotropin receptor, thyroglobulin, gluten, TSHR protein, glutamate decarboxylase 2, receptor-type tyrosine-protein phosphatase-like N, glucose-6-phosphatase 2, insulin isoform 2, zinc transporter 8, glutamate decarboxylase 1, GAD65, UniProt:A2RGM0, integrin alpha-Iib, integrin beta-3, EBV DNA polymerase catalytic subunit, 2′3′-cyclic-nucleotide 3′ phosphodiesterase, myelin associated oligodendrocyte basic protein, small nuclear ribonucleoprotein, U1 small nuclear ribonucleoprotein, histone H2B, histone H2A, histone H3.2, beta-2-glycoprotein, histone H4, 60S ribosomal protein L7, TNF-alpha, myeloperoxidase, Cbir1, MS4A12, DNA topoisomerase, CYP2D6, O-phosphoseryl-tRNA selenium transferase, pyruvate dehydrogenase complex, spectrin alpha chain, steroid 21-hydroxylase, acetylcholine receptor, MMP-16, keratin associated proteins, Chondroitin sulfate proteoglycan 4, myeloblastin, U1 small nuclear ribonucleoprotein 70 kDa, blood group Rh(D), blood group Rh(CE), myelin P2 protein, peripheral myelin protein 22, myelin protein P0, S-arrestin, collagen Alpha-1, coagulation factor VIII, collagen alpha-3(IV), desmoglein-3, desmoglein-1, Insulin-2, major DNA-binding protein, tyrosinase, 5,6-dihydroxyindole-2-carboxylic acid oxidase, HLA-A2, aquaporin-4, myelin proteolipid protein, ABC transporter, HLA I B-27 alpha chain, HLA I B-7 alpha chain, retinol-binding protein 3, or antigenic fragments thereof.

In some embodiments, the non-native protein or peptide is an antigen that is associated with an autoimmune disease. In some embodiments, the non-native protein or peptide is associated with multiple sclerosis, psoriasis, celiac disease, diabetes mellitus Type I, rheumatoid arthritis, systemic lupus erythematosus, inflammatory bowel disease, Graves' disease, Hashimoto's autoimmune thyroiditis, vitiligo, rheumatic fever, pernicious anemia/atrophic gastritis, alopecia areata, immune thrombocytopenic purpura, temporal arteritis, ulcerative colitis, Crohn's disease, scleroderma, antiphospholipid syndrome, autoimmune hepatitis type 1, primary biliary cirrhosis, Sjogren's syndrome, Addison's disease, dermatitis herpetiformis, Kawasaki disease, sympathetic ophthalmia, HLA-B27 associated acute anterior uveitis, primary sclerosing cholangitis, discoid lupus erythematosus, polyarteritis nodosa, CREST Syndrome, myasthenia gravis, polymyositis/dermatomyositis, Still's disease, autoimmune hepatitis type 2, Wegener's granulomatosis, mixed connective tissue disease, microscopic polyangiitis, autoimmune polyglandular syndrome, Felty's syndrome, autoimmune hemolytic anemia, chronic inflammatory demyelinating polyneuropathy, Guillain-Barre Syndrome, Behcet's disease, autoimmune neutropenia, bullous pemphigoid, essential mixed cryoglobulinemia, linear morphea, autoimmune polyglandular syndrome 1 (APECED), acquired hemophilia A, Batten disease/neuronal ceroid lipofuscinoses, autoimmune pancreatitis, Hashimoto's encephalopathy, Goodpasture's disease, pemphigus vulgaris, autoimmune disseminated encephalomyelitis, relapsing polychondritis, Takayasu arteritis, Churg-Strauss syndrome, epidermolysis bullosa acquisita, cicatricial pemphigoid, pemphigus foliaceus, autoimmune hypoparathyroidism, autoimmune hypophysitis, autoimmune inner ear disease, autoimmune lymphoproliferative syndrome, autoimmune oophoritis, autoimmune orchitis, autoimmune polyglandular syndrome, Cogan's syndrome, encephalitis lethartica, erythema elevatum diutinum, Evans syndrome, immunodysregulation polyendocrinopathy enteropathy X-linked (IPEX), Issac's syndrome/acquired neuromyotonia, Miller Fisher syndrome, Morvan's syndrome, PANDAS, POEMS syndrome, Rasmussen's encephalitis, stiff-person syndrome, Vogt-Koyanagi-Harada syndrome, neuromyelitis optica, graft vs host disease, esophageal encephalitis, or autoimmune uveitis.

In some embodiments the non-native protein or peptide is myelin oligodendrocyte glycoprotein, or an antigenic fragment thereof, which is associated with multiple sclerosis (MS). In some embodiments, the non-native protein or peptide is a pancreatic antigen, or antigenic fragment thereof, that is associated with Type I Diabetes (e.g., insulin)

In some embodiments, the heterologous antigen is an antigen, or antigenic fragment thereof, associated with a proliferative disorder such as cancer. In some embodiments the heterologous antigen is associated with melanoma, basal cell carcinoma, squamous cell carcinoma, or testicular cancer. In some embodiments, the heterologous antigen is a melanocyte-specific antigen such as PMEL, TRP2, or MART-1. In some embodiments, the heterologous antigen is a testis cancer antigen such as NY-ESO or MAGE-A. In some embodiments, the heterologous antigen is a neoantigen. In some embodiments, the heterologous antigen is not a neoantigen.

In some embodiments, the heterologous antigen is a protein or antigenic peptide fragment thereof that is not natively expressed by either a commensal bacteria or a host. In some embodiments, the heterologous antigen is gluten, or an antigenic fragment thereof, which is associated with celiac disease in a host.

### Neoantigens

In some embodiments, the non-native protein or peptide is a neoantigen protein or peptide fragment thereof. Neoantigens are mutated peptide antigens that are specifically expressed by cancer cells and are not expressed by normal, healthy cells. A cancerous cell can express a single neoantigen or multiple neoantigens. Some neoantigens are common in various cancers and expressed by a significant number of patients, other neoantigens are rare and expressed by only a few patients. T cells can recognize neoantigens when they are displayed on MHCs of the cancer cell or by an APC. A description of neoantigen repertoire, identification, and their role in cancer immunotherapy is provided in “Neoantigens in cancer immunotherapy.” TN Schumacher et al., Science, 2015: Vol. 348, Issue 6230, pp. 69-74, DOI: 10.1126/science.aaa4971, hereby incorporated by reference in its entirety.

In some embodiments, the neoantigen is associated with a proliferative disorder. In some embodiments, the proliferative disorder is cancer. In some embodiments, the neoantigen is associated with a cancer selected from the group consisting of melanoma, kidney, hepatobiliary, head-neck squamous carcinoma (HNSC), pancreatic, colon, bladder, glioblastoma, prostate, lung, breast (mammary), ovarian, gastric, kidney, bladder, esophageal, renal, melanoma, leukemia, lymphoma, mesothelioma, basal cell carcinoma, squamous cell carcinoma, and testicular cancer.

In some embodiments, the neoantigen is selected from the group consisting of Ints11, Kif18 bp, T3 sarcoma neoantigens, and a neoantigen as expressed by the TRAMPC2 prostate cancer cell line.

Ints11 and Kif18 bp neoantigens are described in Castle et al., “Exploiting the Mutanome for Tumor Vaccination” Cancer Res. 2012; 72(5):1081-1091; T3 sarcoma neoantigens neoantigens are described in Alspach et al., “MHC-II neoantigens shape tumour immunity and response to immunotherapy” Nature. 2019; 574:696-701; Tramp-C2 neoantigens are described in Fasso et al., “SPAS-1 (stimulator of prostatic adenocarcinoma-specific T cells)/SH3GLB2: A prostate tumor antigen identified by CTLA-4 blockade” PNAS. 2008; 105(9):3509-3514, each of which are incorporated by reference.

Neoantigens and tumor-associated peptides that can serve as active pharmaceutical ingredients of vaccine compositions that stimulate an antitumor response are described in U.S. Pat. No. 9,115,402, which is herein incorporated by reference in its entirety. In certain embodiments, a neoantigen can be selected by first identifying the available mutations that constitute a neoantigen or tumor-associated antigen in cancer cells from an individual cancer subject. In certain embodiments, once identified, the neoantigen, or immunogenic fragment thereof, can be expressed in a live, recombinant commensal bacterium described herein to elicit an adaptive T cell response in the cancer subject or in HLA-matched donor T cells that can be introduced into the cancer subject to recognize and kill the cancer cells.

### Infectious Disease Antigens

In some embodiments, the at least one non-native protein or peptide is an antigen associated with an infectious disease-causing organism. In certain embodiments, an infectious disease causing organism includes any infectious virus, bacteria, fungus, or parasite that infects and causes disease in a host. In some embodiments, the host is a mammal. In some embodiments, the host is a human. In some embodiments, the infectious disease-causing organism is a virus. In some embodiments, the infectious disease-causing organism is a bacteria. In some embodiments, the infectious disease-causing organism is a fungus. In some embodiments, the infectious disease-causing organism is a parasite.

In some embodiments, the infectious disease-causing organism is selected from the group consisting of: Influenza virus A, Influenza virus B, Influenza virus C, herpesviruses, herpes simplex virus (HSV-1, HSV-2), retroviruses, human immunodeficiency virus (HIV-1, HIV-2), human adenovirus (hAdV), parainfluenza viruses (PIV), respiratory syncytial virus (RSV), rhinoviruses, coronaviruses, SARS-coronavirus, COVID-19, measles virus, mumps virus, rubella virus, polio virus, varicella-zoster virus (VZV), dengue virus, flaviviruses, ebola virus, Epstein-Barr virus, norovirus, rotavirus, hepatitis A virus, hepatitis B virus, hepatitis C virus, West Nile virus, rabies virus, Staphylococcus aureus (MRSA), Neisseria gonorrhoeae, Chlamydia trachomatis, Treponema pallidum, Clostridium tetani, Clostridium difficile, Mycobacterium tuberculosis, Borrelia burgdorferi, Yersinia pestis, Bordetella pertussis, Vibrio cholerae, Bacillus anthracis, Clostridium botulinum, group A Streptococcus bacteria (strep throat causing bacteria), Listeria, Shigella, Streptococcus pneumoniae, Mycoplasma pneumoniae, Haemophilus influenzae, Legionella pneumophila, Cryptococcus, Histoplasmosis, Pneumocystis jirovecii, Aspergillus, Trichophyton, Microsporum, Epidermophyton, Trichomonas vaginalis, Plasmodium, Toxoplasma gondii, Giardia lamblia, and Leishmania. In some embodiments, the at least one non-native protein or peptide is NP366-374, NP306-322, NA177-193, M2 ectodomain, HA2 stem-HA 12-63, HA2 stem-HA 76-130, gB glycoprotein, gd glycoprotein, and gB glycoprotein 498-505.

In some embodiments, the non-native protein or peptide comprises an amino acid sequence as listed in Table 3.

TABLE 3
EXEMPLARY NON-NATIVE PEPTIDES
AND AMINO ACID SEQUENCES.

| | SEQ ID |
|---|---|
| Antigen | NO. |
| OVA 257-264 (OT-1) | 1 |
| OVA 323-329 (OT-2) | 2 |
| OT3pep | 3 |
| MOG 35-55 | 4 |
| Insulin B9-23 (R22) | 5 |
| epitope | |
| ChgA epitope | 6 |
| 2.5HIP epitope | 7 |
| PLP epitope 1 | 8 |
| PLP epitope 2 | 9 |
| PLP epitope 3 | 10 |
| PLP epitope 4 | 11 |
| MBP epitope | 12 |
| Villin epitope 1 | 13 |
| Villin epitope 2 | 14 |
| Villin epitope 3 | 15 |
| Epcam epitope | 16 |
| NP 366-374 epitope | 17 |
| NP 306-322 epitope | 18 |
| NA 177-193 epitope | 19 |
| M2e epitope | 20 |
| HA2 12-63 epitope | 21 |
| HA2 76-130 epitope | 22 |
| HSV gB glycoprotein | 23 |
| HSV gd glycoprotein | 24 |
| HSV gB glycoprotein | 25 |
| 498-505 | |
| SARS-COV2 Spike | 26 |
| protein epitope | |
| HIV-gp120 epitope | 27 |
| HIV-gp41 epitope | 28 |
| HIV V1V2 apex epitope | 29 |
| HIV V3 loop | 30 |
| HIV CD4 binding site | 31 |
| HIV MPER | 32 |

### APC-Targeting Moieties

In some embodiments, engineered microorganisms, or surface-labeled bacteria, e.g., live, recombinant commensal bacteria, are engineered to express, or display, a non-native protein or peptide that includes an APC-targeting moiety. In certain embodiments, non-native proteins or peptides that include an APC-targeting moiety can also include one or more antigenic sequences. In certain embodiments, non-native proteins or peptides that include both an APC-targeting moiety and one or more antigenic sequences can also be engineered to be secreted into the extracellular space. In certain embodiments, non-native proteins or peptides that include both an APC-targeting moiety and one or more antigenic sequences can also be engineered to be tethered to the cell wall of the engineered microorganism or surface-labeled bacteria. In certain embodiments, secretion and cell-wall tethering are described further in the section titled “Signal Sequence Peptides” herein.

In some embodiments, an engineered microorganism is engineered to express, or a surface-labeled bacterium displays, a first non-native protein or peptide that includes an APC-targeting moiety and a second distinct non-native protein or peptide that includes one or more antigenic sequences.

In certain embodiments, APC-targeting moieties include, but are not limited to, an antibody or antigen-binding fragment thereof, such as a Fab fragment, a Fab′ fragment, a single chain variable fragment (scFv), a single domain antibody (sdAb) either as single specific or multiple specificities linked together (e.g., camelid antibody domains), or full-length single-chain antibody (e.g., full-length IgG with heavy and light chains linked by a flexible linker). In certain embodiments, an APC-targeting moiety can be an antigen-binding fragment capable of expression and proper post-translational processing such that the antigen-binding fragment is capable of binding a cognate antigen. In certain embodiments, an APC-targeting moiety can be a single-domain antibody (e.g., a camelid antibody) or antigen-binding fragment thereof. An APC-targeting moiety can be the variable domain of a single-domain antibody (VHH, also referred to as a “nanobody”).

In certain embodiments, an APC-targeting moiety can include the VHH sequence of SEQ ID NO:33. In certain embodiments, an APC-targeting moiety can include the VHH sequence of SEQ ID NO:34. In certain embodiments, an APC-targeting moiety can include each of the CDRs of VHH sequence SEQ ID NO:33. In certain embodiments, an APC-targeting moiety can include each of the CDRs of VHH sequence SEQ ID NO:34. In certain embodiments, an APC-targeting moiety can include the CDR3 of VHH sequence SEQ ID NO:33. In certain embodiments, an APC-targeting moiety can include the CDR3 of VHH sequence of SEQ ID NO:34.

In certain embodiments, APC-targeting moieties can bind to (“target”) any cognate ligand associated with an APC, such as any cellular marker associated with dendritic cells, macrophages, Langerhans cells, B cells, intestinal epithelial cells, and innate lymphoid cells, splenic dendritic cells, CD8+ dendritic cells, CD11b+ dendritic cells, plasmacytoid dendritic cells, follicular dendritic cells, monocytic cells, macrophages, bone marrow-derived macrophages, or Kupffer cells. In some embodiments, an APC-targeting moiety targets any cellular marker associated with a CD103+CD11b+ dendritic cell. In some embodiments, an APC-targeting moiety targets any cellular marker associated with a CX3CR1+ intestinal macrophage. In some embodiments, an APC-targeting moiety targets CD11b. In some embodiments, an APC-targeting moiety targets CD11b or an MHC-II targeting moiety.

### Signal Sequence Peptides

In some embodiments, engineered microorganisms, or surface-labeled bacteria, e.g., live, recombinant commensal bacteria, are engineered to express, or display, a non-native protein or peptide that includes a signal sequence peptide.

In some embodiments, signal sequence peptides direct tethering of the fusion protein to a cell wall of the bacterium following expression. In certain embodiments, the signal sequence peptide can include a sortase-derived signal sequence peptide. Signal sequence peptides that direct tethering can be derived from an endogenous gene of the engineered microorganism or surface-labeled bacterium. In certain embodiments, signal sequence peptides that direct tethering can be a sequence heterologous to the engineered microorganism or surface-labeled bacterium, such as a paralog. In certain embodiments, an engineered microorganism, or surface-labeled bacterium can be S. epidermidis and a signal sequence peptide can be derived from S. aureus. In certain embodiments, Signal sequence peptides that direct tethering can be signal sequence peptides derived from proteins that are substrates of sortase (e.g., Protein A of S. aureus).

In general, proteins to be tethered to a cell wall typically include a cell wall spanning peptide domain. Cell wall spanning peptide domains can be derived from an endogenous gene of the engineered microorganism, or surface-labeled bacterium. Cell wall spanning peptide domains can be a sequence heterologous to the engineered microorganism, or surface-labeled bacterium, such as a paralog. In certain embodiments, an engineered microorganism, or surface-labeled bacterium, can be S. epidermidis and a cell wall spanning peptide domain can be derived from S. aureus. In certain embodiments, cell wall spanning peptide domains can be derived from proteins that are substrates of sortase (e.g., Protein A of S. aureus).

In certain embodiments, a general organization for a protein to be tethered to a cell wall can include a signal sequence peptide that directs tethering positioned N-terminal of a non-native protein or peptide and a cell wall spanning peptide domain positioned C-terminal of the non-native protein or peptide.

In some embodiments, signal sequence peptides direct secretion of the fusion protein from the bacterium (i.e., into the extracellular space) following expression. In certain embodiments, signal sequence peptides promoting secretion include, but are not limited to, a twin arginine translocation (tat) signal sequence peptide or a general secretion (sec) signal sequence peptide. In certain embodiments, a signal sequence peptide promoting secretion can be a tat signal sequence peptide. In certain embodiments, signal sequence peptides promoting secretion can be derived from an endogenous gene of the engineered microorganism or surface-labeled bacterium. In certain embodiments, signal sequence peptides promoting secretion can be a sequence heterologous to the engineered microorganism, or the surface-labeled bacterium, such as a paralog. In certain embodiments, an engineered microorganism can be S. epidermidis and a signal sequence peptide promoting secretion can be derived from S. aureus (e.g., the signal sequence peptide from fepB). In certain embodiments, a signal sequence peptide promoting secretion can be a sec signal sequence peptide. In certain embodiments, signal sequence peptides include predicted signal sequence peptides such as the signal sequence peptide derived from predicted sec-secreted S. epidermidis protein (gene locus HMPREF9993_06668).

### 6. Nucleic Acids

In some embodiments, the modified microorganism, e.g., a live, recombinant commensal bacterium, comprises a non-native or heterologous nucleic acid that is used to express a non-native protein or peptide, or heterologous antigen or antigenic fragment thereof. In some embodiments, the heterologous nucleic acid is an RNA that is translated to produce a heterologous protein, or antigenic fragment thereof. In some embodiments, the heterologous nucleic acid is a DNA that encodes a heterologous protein, or antigenic fragment thereof (i.e., the DNA can be transcribed into mRNA that is translated to produce the heterologous protein or antigenic fragment thereof).

In certain embodiments, the heterologous nucleic acid typically includes regulatory sequences and coding region sequences. In some embodiments, the regulatory sequences are operably linked to the coding region sequences, such that the regulatory sequences control expression (e.g., transcription or translation) of the coding region sequences. In certain embodiments, the regulatory sequences can include sequence elements such as promoters and enhancers that bind regulatory proteins such as transcription factors and influence the rate of transcription of operably linked sequences. In certain embodiments, the regulatory sequences can be located upstream (5′) or downstream (3′) of the coding region sequences, or both.

In some embodiments, the coding region sequences encode a heterologous protein that is useful for eliciting an immune response in a mammal. As is known by persons of skill in the art, various online servers can used to predict epitope-coding sequences that strongly bind to MHC-II and elicit a T cell response (for example, see Reynisson et al. NetMHCpan-4.1 and NetMHCIIpan-4.0: improved predictions of MHC antigen presentation by concurrent motif deconvolution and integration of MS MHC eluted ligand data. Nucleic Acids Res. 2020; 48(W1):W449-454.). In certain embodiments, the nucleic acid can also include sequences that, when transcribed and translated, provide signals for trafficking the heterologous protein to a specific cellular location or compartment (e.g., intracellular, secreted, or membrane bound).

In some embodiments, the heterologous nucleic acid is an expression vector comprising regulatory sequences that upregulate or downregulate transcription of the coding region sequence into RNA. In some embodiments, the modified microorganism comprises the necessary components to translate the RNA into protein, such as amino acids and tRNA. In some embodiments, the modified microorganism is a live recombinant commensal bacterium that comprises the necessary components to translate the RNA into protein, such as amino acids and tRNA. In certain embodiments, the expression vector can contain regulatory elements that direct expression of the heterologous antigen anywhere in the live, recombinant commensal bacterium. In certain embodiments, the expression vector can contain regulatory elements that direct expression of the heterologous antigen in the cytoplasm (i.e., soluble, not in inclusion bodies), periplasm, fused to a cell surface protein, or secreted by the bacterium. Nucleic acid vectors for the expression of recombinant proteins in bacteria are well known by persons of skill in the art. In some embodiments, the expression vector is pNBU2-bla-ermGb, pNBU2-bla-tetQb, or pExchange-tdk (see, e.g., Wang J. et al. (2000). J Bacteriol. 182. 3559-71; pMM668, Addgene; Mimee M. et al. (2015) Cell Syst. 1(1):62-71; and Koropatkin N. et al. 2008. Structure. 16(7): 1105-1115).

In some embodiments, the expression vector is a pWW3837 vector (Genbank #KY776532), which is used to integrate an antigenic epitope coding region into the bacterial genome, as described in Whitaker et al., “Tunable Expression Tools Enable Single-Cell Strain Distinction in the Gut Microbiome,” Cell 169, 538-546, Apr. 20, 2017.

In some embodiments, the heterologous nucleic acid is stably integrated into the genome of the bacteria. In some embodiments, the heterologous nucleic acid is maintained as a plasmid in the bacteria. In some embodiments, the heterologous nucleic acid is an episomal plasmid.

In some embodiments, the heterologous nucleic acid comprises an epitope coding region sequence as listed in Table 4.

TABLE 4
EXEMPLARY NON-NATIVE PEPTIDE AND PROTEIN
CODING REGION SEQUENCES.

| | SEQ ID |
|---|---|
| Antigen | NO. |
| OVA 257-264 (OT-I) | 35 |
| OVA 323-329 (OT-II) | 36 |
| OT3 pep | 37 |
| MOG 35-55 | 38 |
| Insulin B9-23 (R22) epitope | 39 |
| ChgA epitope | 40 |
| 2.5HIP epitope | 41 |
| PLP epitope 1 | 42 |
| PLP epitope 2 | 43 |
| PLP epitope 3 | 44 |
| PLP epitope 4 | 45 |
| MBP epitope | 46 |
| Villin epitope 1 | 47 |
| Villin epitope 2 | 48 |
| Villin epitope 3 | 49 |
| Epcam epitope | 50 |
| NP 366-374 epitope | 51 |
| NP 306-322 epitope | 52 |
| NA 177-193 epitope | 53 |
| M2e epitope | 54 |
| HA2 12-63 epitope | 55 |
| HA2 76-130 epitope | 56 |
| HSV gB glycoprotein | 57 |
| HSV gd glycoprotein | 58 |
| HSV gB glycoprotein 498-505 | 59 |
| SARS-COV2 Spike protein epitope | 60 |

In some embodiments, the heterologous nucleic acid comprises non-natural nucleotides or analogues of natural nucleotides. Nucleotide analogs or non-natural nucleotides include nucleotides containing any type of modification to a base, sugar or phosphate moiety. Modifications can include chemical modifications. In certain embodiments, modifications can be of the 3′OH or 5′OH groups of the backbone, sugar component or nucleotide base. In certain embodiments, modifications may include the addition of non-naturally occurring linker molecules and/or cross-strand or intra-strand crosslinks. In certain embodiments, a modified nucleic acid comprises modification of one or more of a 3′OH or 5′OH group, backbone, sugar component, or nucleotide base, and/or addition of a non-naturally occurring linker molecule. In certain embodiments, the modified skeleton includes a skeleton other than the phosphodiester skeleton. In one aspect, modified sugars include sugars other than deoxyribose (in modified DNA) or sugars other than ribose (in modified RNA). In certain embodiments, modified bases include bases other than adenine, guanine, cytosine or thymine (in modified DNA) or bases other than adenine, guanine, cytosine or uracil (in modified RNA).

### 7. Methods of Producing Live, Recombinant Commensal Bacteria

In certain embodiments, commensal bacteria can be engineered to express, or surface-labeled to display, non-native proteins or peptides, or heterologous antigens or antigenic fragments thereof, using general molecular biology methods as described in Green, M. R. and Sambrook, J., eds., Molecular Cloning: A Laboratory Manual, 4^{th} ed., Cold Spring Harbor Laboratory Press, Cold Spring Harbor, N.Y. (2012), and Ausubel, F. M., et al. Current Protocols in Molecular Biology (Supplement 99), John Wiley & Sons, New York (2012), which are incorporated herein by reference.

In certain embodiments, to produce a live, recombinant, commensal bacterial strain that expresses a non-native protein or peptide, or heterologous antigen or antigenic fragment thereof, antigenic epitope coding sequences can be cloned into an expression vector. In certain embodiments, a representative expression vector is the pWW3837 vector (Genbank #KY776532), (see Whitaker et al., “Tunable Expression Tools Enable Single-Cell Strain Distinction in the Gut Microbiome,” Cell 169, 538-546, Apr. 20, 2017). In certain embodiments, the antigenic epitope coding sequences can be cloned into the expression vector by known methods such as Gibson assembly. In certain embodiments, the expression vector can then be electroporated into a suitable bacterial donor strain, such as an Escherichia coli S17 lambda pir donor strain. In certain embodiments, the E. coli donor strain can be co-cultured overnight with recipient live commensal bacteria for conjugation, and positive colonies screened for incorporation of the expression vector.

In certain embodiments, expression of the non-native protein or peptide or heterologous antigen can be determined by various assays, including detecting expression of the RNA encoding the antigen. In certain embodiments, the assay is Northern analysis, RT-PCR, or protein expression detection. In certain embodiments, the protein expression detection is Western analysis.

### 8. Pharmaceutical Compositions

In some embodiments, provided in the present disclosure are pharmaceutical compositions comprising a modified microorganism as described herein and a pharmaceutically acceptable carrier. In some embodiments, provided in the present disclosure are pharmaceutical compositions comprising a modified microorganism that is a live, recombinant commensal bacterium, as described herein and a pharmaceutically acceptable carrier. In some embodiments, the pharmaceutical composition induces an antigen-specific T cell response to a heterologous antigen expressed by the modified microorganism described herein when ingested by, or otherwise administered to, a subject. In some embodiments, the composition induces an antigen-specific T_{reg} response to the heterologous antigen expressed by the modified microorganism described herein. In some embodiments, the composition induces an antigen-specific T_{eff} response to the heterologous antigen expressed by the modified microorganism described herein.

In some embodiments, the pharmaceutical composition comprises a modified microorganism comprising a non-native or heterologous nucleic acid that encodes a non-native or heterologous antigen that induces an antigen-specific T cell response when the composition is administered to a subject. In some embodiments, the pharmaceutical composition comprises a modified microorganism comprising a heterologous nucleic acid that encodes a heterologous antigen that induces an antigen-specific T_{reg} response when the composition is administered to a subject. In some embodiments, the heterologous antigen is capable of being tethered to the bacterial cell surface. In some embodiments, the pharmaceutical composition comprises a modified microorganism comprising a heterologous nucleic acid that encodes a heterologous antigen that induces an antigen-specific T_{eff} response when the composition is administered to a subject. In some embodiments, the heterologous antigen is capable of being tethered to the bacterial cell surface.

In some embodiments, the pharmaceutical composition comprises a live, recombinant commensal bacterium comprising a non-native or heterologous nucleic acid that encodes a non-native or heterologous antigen that induces an antigen-specific T cell response when the composition is administered to a subject. In some embodiments, the pharmaceutical composition comprises a modified commensal bacterium comprising a heterologous nucleic acid that encodes a heterologous antigen that induces an antigen-specific T_{reg} response when the composition is administered to a subject. In some embodiments, the heterologous antigen is capable of being tethered to the bacterial cell surface. In some embodiments, the pharmaceutical composition comprises a modified commensal bacterium comprising a heterologous nucleic acid that encodes a heterologous antigen that induces an antigen-specific T_{eff} response when the composition is administered to a subject. In some embodiments, the heterologous antigen is capable of being tethered to the bacterial cell surface.

In certain embodiments, the pharmaceutical compositions described herein can include a pharmaceutically acceptable excipient. In certain embodiments, examples of pharmaceutically acceptable excipients include, without limitation, sterile solutions such as water, saline, and phosphate buffered solutions. In certain embodiments, additional examples of pharmaceutical excipients are described in the Handbook of Pharmaceutical Excipients, 8^{th} Edition, Authors/Editor: Sheskey, Paul J.; Cook, Walter G.; Cable, Colin G., Pharmaceutical Press (ISBN: 978-0-857-11271-2). It will be understood that the type of excipient used will depend on the route of administration to a subject.

In some embodiments, the pharmaceutical composition comprises a modified bacterium that is derived from a commensal bacterium that is native to the digestive tract of a mammal. In some embodiments, the pharmaceutical composition comprises a live, recombinant commensal bacterium selected from a Bacteroides sp. Or Helicobacter sp. In some embodiments, the pharmaceutical composition comprises a recombinant B. thetaiotaomicron, B. vulgatus, B. finegoldii or H. hepaticus.

In some embodiments, the pharmaceutical composition comprises a modified bacteria that is derived from a commensal bacteria that is native to the skin of a mammal. In some embodiments, the pharmaceutical composition comprises a Staphylococcus spp. In some embodiments, the pharmaceutical composition comprises a recombinant S. epidermidis.

In certain embodiments, the pharmaceutical composition disclosed herein can be administered to a subject via a suitable route that induces an antigen-specific immune response to the heterologous antigen, such as oral, nasal, subcutaneous, dermal, intradermal, intramuscular, mucosal or rectal.

In some embodiments, the pharmaceutical composition disclosed herein is administered to a subject via a suitable route to allow the modified microorganism to colonize a niche in the subject that the microorganism from which the modified microorganism was derived would natively inhabit. In some embodiments, the pharmaceutical composition disclosed herein is orally administered to a subject to allow a modified microorganism to colonize the host's gastrointestinal tract. In some embodiments, the pharmaceutical composition disclosed herein is topically administered to a subject to allow a modified microorganism to colonize the host's skin.

In some embodiments, the pharmaceutical composition disclosed herein is administered to a subject via a suitable route to allow the modified microorganism is a live, recombinant commensal bacterium that colonizes a niche in the subject that the microorganism from which the modified microorganism was derived would natively inhabit. In some embodiments, the pharmaceutical composition disclosed herein is orally administered to a subject to allow a modified microorganism that is a live recombinant bacterium derived from a commensal bacterium native to the gastrointestinal tract of the subject, to colonize the host's gastrointestinal tract. In some embodiments, the pharmaceutical composition disclosed herein is topically administered to a subject to allow a modified microorganism that is a live recombinant bacterium derived from a commensal bacterium native to the skin of the subject, to colonize the host's skin.

In some embodiments, the pharmaceutical composition comprises a material, such as a delayed-release enteric coating, that permits transit through the stomach to the small intestine before the modified microorganisms described herein are released. In some embodiments, the pharmaceutical composition disclosed herein comprises an enteric-coated capsule containing a modified microorganism described herein. In some embodiments, the enteric coating comprises a polymer that is stable at an acidic pH, such as the acidic pH of the stomach, but breaks down or dissolves rapidly at an alkaline pH, such as the pH in the small intestine (pH 7-9).

In some embodiments, the pharmaceutical composition comprises a material, such as a delayed-release enteric coating, that permits transit through the stomach to the small intestine before the modified microorganisms that are recombinant commensal bacteria, are released. In some embodiments, the pharmaceutical composition disclosed herein comprises an enteric-coated capsule containing a modified microorganism that is a live, recombinant commensal bacterium, described herein. In some embodiments, the enteric coating comprises a polymer that is stable at an acidic pH, such as the acidic pH of the stomach, but breaks down or dissolves rapidly at an alkaline pH, such as the pH in the small intestine (pH 7-9).

In some embodiments, the pharmaceutical composition can further comprise additional agents that are useful for treating a disease or pathological condition in a subject. In certain embodiments, examples of additional agents include small molecule drugs or antibodies that are useful for treating a disease or pathological condition in a subject.

### 9. Synthetic Bacterial Communities Comprising Bacteria that Induce an Adaptive T Cell Response

In certain embodiments, modified microorganisms produced according to the disclosure (e.g., including but not limited to a live recombinant commensal bacterium) may be administered to a subject to induce an antigen-specific T cell immune response. In certain embodiments, it will be recognized that administering a bacterium does not generally refer to administration of a single bacterial cell, but encompasses administering a plurality of bacterial cells, typically a clonal population of bacterial cells with a desired property (i.e., expression of a heterologous antigen or antigenic fragment thereof).

A “high-complexity defined microbial community,” as used herein, refers to a physical combination of a plurality of different microorganisms (e.g., a plurality of different bacterial strains), wherein each microbial strain has been molecularly defined.

U.S. Provisional Application No. 62/770,706, filed Nov. 21, 2018, and related International Patent Application No. PCT/US2019/062689, which published on May 28, 2020 as WO2020106999A1, both entitled “High Complexity Synthetic Gut Bacterial Communities”, and the content of each of which is herein incorporated by reference in its entirety, describe defined stable microbial communities produced using in vitro and in vivo back-fill methods, i.e., “back-fill communities,” and methods for making such communities. In certain embodiments, these microbial communities comprise at least one or more microbial cell of interest and are stable when engrafted into the mammalian (e.g., human) gut, such as a gut containing a human microbiome in the sense that the microbial ecosystem is at homeostasis such that the at least one or more microbial cell of interest does not drop out of the community, is not over-grown by competing microbes in the gut, and does not overgrow and displace other microbes in the gut. If the combination of strains in the population is unstable, the population may change in unpredictable ways, which may change the metabolic phenotype of the community.

U.S. Provisional Application No. 62/770,706, and related International Patent Application No. PCT/US2019/062689, describe generation, screening and engraftment of communities with a desired metabolic phenotype. In certain embodiments, a metabolic phenotype may be the ability of a microbial strain or microbial community to transform one or more first compounds into one or more second compounds. In certain embodiments, a first compound(s) is enzymatically converted by the microbe or community into a second compound(s), and the metabolic phenotype is an increase in the amount of the second compound(s).

In some embodiments, a modified microorganism as described herein, e.g., including but not limited to a live, recombinant commensal bacterium, can be administered in combination with a high-complexity defined microbial community. In some embodiments, the bacterium is administered to the host in combination with a high-complexity defined microbial community, and the high-complexity defined microbial community promotes a T_{H}2, T_{reg}, and/or T_{H}17 response in the host. In some embodiments, a modified microorganism as described herein, e.g., including but not limited to a live, recombinant commensal bacterium, can be administered in combination with a high-complexity defined microbial community as disclosed in International Application No. PCT/US2019/062689. In certain embodiments, a desired phenotype of a high-complexity defined microbial community is the ability of a live, recombinant commensal bacterial cell as disclosed herein, to expresses a heterologous antigen, or antigenic fragment thereof, in sufficient amounts to induce an antigen-specific T cell response to the heterologous antigen. In certain embodiments, a high-complexity defined microbial community comprising a modified microorganism, e.g., a live recombinant commensal bacterium, is administered to a subject to allow colonization of a niche in the subject that a commensal bacterium from which the recombinant bacterium was derived would natively inhabit, resulting in induction of an antigen-specific T cell response to the heterologous antigen, or antigenic fragment thereof, expressed by the live recombinant commensal bacterium. In some embodiments, a high-complexity defined microbial community comprising a live, recombinant commensal bacterium described herein induces an antigen-specific regulatory T cell response in the subject into which the community is engrafted. In some embodiments, a high-complexity defined microbial community comprising a live, recombinant commensal bacteria described herein, induces an antigen-specific T effector cell response in the subject into which the community is engrafted.

One of ordinary skill in the art will appreciate that a high-complexity defined microbial community capable of inducing an antigen-specific T cell response to a heterologous antigen can be produced as described in International Application No. PCT/US2019/062689, with the modification that the metabolic phenotype is the ability to elicit an antigen-specific T cell response. As disclosed therein, cultured or in vivo backfill communities were assayed for the ability to induce the desired antigen-specific T cell response. In certain embodiments disclosed therein, the desired antigen-specific T cell response may be considered a type of metabolic phenotype.

Assays for an metabolic phenotype are known in the art and are described in this disclosure including, without limitation, assays described in the section of this disclosure entitled “Methods for Detecting a T Cell Response.”

### 10. Methods of Inducing an Antigen-Specific T Cell or B Cell Response

In another aspect, provided are methods for inducing an antigen-specific T cell or B cell response to a non-native protein, peptide, antigen, or antigenic fragment thereof, expressed or displayed by a modified microorganism, e.g. a live, recombinant commensal bacterium, as described herein. In certain embodiments, the methods can be performed in vitro or in vivo.

### T Cells

In some embodiments, the T cell response is a T_{H}1, T_{H}2, T_{H}17, T_{reg}, CD8⁺, or T Follicular helper (T_{FH}) response. In some embodiments, the live, recombinant commensal bacterium limits differentiation of T_{H}1 T cells in the host. In some embodiments, the bacterium modulates the native host niche to limit differentiation of T_{H}1 T cells in the host. In some embodiments, the bacterium promotes differentiation of T_{H}2 T cells in the host. In some embodiments, the bacterium modulates the native host niche to promote differentiation of T_{H}2 T cells in the host.

In certain embodiments, a T cell response after administration of a modified bacterium as described herein can include cytokine and/or chemokine expression, or cell killing. In some embodiments, the T cell response comprises a cytokine and/or chemokine response. In some embodiments, the T cell response comprises increased secretion of cytokines and/or chemokines. Increased secretion of cytokines and/or chemokines includes, but is not limited to, an increase in the number of T cells secreting cytokines and/or chemokines as compared to the administration of a non-modified bacterium; an increase in the amount or volume of secreted cytokines and/or chemokines as compared to the administration of a non-modified bacterium; enhanced secretion of cytokines and/or chemokines by T cells as compared to the administration of a non-modified bacterium; or an induction of the secretion of cytokines and/or chemokines as compared to the administration of a non-modified bacterium. In some embodiments, the T cell response comprises a T_{H}2 response.

In some embodiments, the T cell response comprises a cytotoxic T cell response. An increased cytotoxic T cell response includes, but is not limited to, an increase in the number of cytotoxic T cells as compared to the administration of a non-modified bacterium; an increase in the activation of cytotoxic T cells as compared to the administration of a non-modified bacterium; enhanced activation of cytotoxic T cells as compared to the administration of a non-modified bacterium; or an induction of cytotoxic T cell activation as compared to the administration of a non-modified bacterium.

In some embodiments, the T cell response does not comprise a T_{H}1 response. In certain embodiments, limiting, suppressing, or reducing a T_{H}1 response, include, but is not limited to, a reduction or decrease in the number of T_{H}1 T cells or activated T_{H}1 T cells as compared to the administration of a non-modified bacterium.

Regulatory T cells (T_{regs}) have pluripotent anti-inflammatory effects on multiple cell types. In particular, they control the activation of innate and adaptive immune cells. T_{regs} acting in an antigen-specific manner reduce effector T cell activation and function, for example, after effector T cells have successfully mounted an attack against an invading pathogen, or to suppress reactivity to self-antigen and thereby prevent autoimmune disease.

T_{reg} cells play a major role in establishing and maintaining immune homeostasis in peripheral tissues, particularly at barrier sites where they stably reside. In the intestinal lamina propria, T_{reg} cells not only maintain self-tolerance but also play a crucial role in mediating tolerance to commensal organisms. A large percentage of gut-resident T_{reg} cells recognize commensal antigens, and thymically derived T_{reg} cells support tolerance to intestinal microbes. In addition, certain bacterial species expand T_{reg} cells in the lamina propria.

T_{regs} are a subset of T helper (T_{H}) cells, and are considered to be derived from the same lineage as naïve CD4+ cells. T_{regs} are involved in maintaining tolerance to self-antigens, and preventing auto-immune disease. T_{regs} also suppress induction and proliferation of effector T cells (T_{eff}). T_{regs} produce inhibitory cytokines such as TGF-β, IL-35, and IL-10. T_{regs} express the transcription factor Foxp3. In humans, the majority of T_{reg} cells are MHC-II restricted CD4+ cells, but there is a minority population that are FoxP3+, MHC-I restricted, CD8+ cells. T_{regs} can also be divided into subsets: “natural” CD4+CD25+ FoxP3+T_{reg} cells (nT_{regs}) that develop in the thymus, and “inducible” regulatory cells (iT_{regs}) which arise in the periphery. Naturally occurring T_{regs} suppress self-reactive immune responses in the periphery. iT_{regs} are also CD4+CD25+ FoxP3+, and develop from mature CD4+ T cells in the periphery (i.e., outside of the thymus) from conventional CD4+ T cells to ensure tolerance to harmless antigens, including those derived from, for example, food and intestinal flora. Both subsets of T_{reg} cells are characterized by expression of high levels of CD25 and the transcription factor Foxp3. T_{regs} are thought to inhibit the antigen-specific expansion and/or activation of self-reactive effector T cells and to secrete suppressive cytokines, including TGF-β or IL-10. iT_{regs} can also express both RORγt and Foxp3. Research has shown that TGF-β and retinoic acid produced by dendritic cells can stimulate naïve T cells to differentiate into T_{regs}, and that naïve T cells within the digestive tract differentiate into T_{regs} after antigen stimulation. iT_{regs} can also be induced in culture by adding TGF-β.

T effector (T_{eff}) cells generally stimulate a pro-inflammatory response upon antigen-specific T Cell receptor (TCR) activation via the expression or release of an array of membrane-bound and secreted proteins that are specialized to deal with different classes of pathogen. T_{eff} cells are usually divided into CD8+ cytotoxic T cells and T helper cells. T helper cells can be further classified as T_{H}1 cells, T_{H}2 cells, and T_{H}17 cells.

CD8+ cytotoxic T cells recognize and kill target cells that display peptide fragments of intracellular pathogens (e.g., viruses) presented in the context of MHC-I molecules at the cell surface. CD8+ cytotoxic T cells store preformed cytotoxins in lytic granules which fuse with the membranes of infected target cells. CD8+ cytotoxic T cells additionally express Fas ligand, which induces apoptosis in Fas-expressing target cells.

T helper (T_{H}) cells are a class of CD4+ cells that function to regulate the proliferation of B cells and B cell responses. T_{H} cells play an important role in humoral immunity and immunopathology. CD4+T helper cells differentiate into either T_{H}1 or T_{H}2 cells. Both T_{H}1 and T_{H}2 cells express CD4 and recognize peptide fragments processed within intracellular vesicles and presented on the cell surface in the context of MHC-II molecules. T_{H}1 cells can directly or indirectly activate a number of other immune cells, including macrophages and B cells, thereby promoting more efficient destruction and clearance of intracellular microorganisms. T_{H}1 cells can also be involved in pathways that lead to activation of CD8+ cytotoxic T cells. T_{H}2 cells stimulate the differentiation of B cells and promote the production of antibodies and other effector molecules of the humoral immune response. T_{H} cells can differentiate into T_{H}1 or T_{H}2 T cells depending upon antigen stimulation and cytokine environment. T helper cells first activated by antigen in the presence of IL-12 develop predominantly into T_{H}1 cells, whereas those activated in the presence of IL-4 develop predominantly into T_{H}2 cells. Progenitor T helper cells may require cellular divisions before becoming competent to synthesize the cytokines that are indicative of either the T_{H}1 or T_{H}2 pathway. T_{H}1 and T_{H}2 cell phenotypes are different from each other in early activation signal transduction pathways, especially in the different roles of TCR-related protein tyrosine kinases. TCR and its downstream protein tyrosine kinases such as Fyn, p56(Ick), and ZAP-70 are involved in the development and differentiation of T_{H}1 and T_{H}2 cells.

T_{H}17 cells are a subset of pro-inflammatory T_{H} cells that express IL-17. T_{H}17 cells are developmentally distinct from T_{H}1 and T_{H}2 cells. The signaling pathway that induces differentiation of T_{H} cells into T_{H}17 cells inhibits T_{reg} differentiation.

T follicular helper cells (T_{FH}) are a subset of CD4+ cells. T_{FH} cells are essential for helping cognate B cells form and maintain the germinal center (GC) reaction, and for development of humoral immune responses. These cells are defined by expression of the chemokine receptor CXCR5, which directs them to the B cell follicles via gradients of the chemokine CXCL131. T_{FH} cells also express the transcription factor Bcl6 (which represses Blimp-1/Prdm1) and high levels of the costimulatory receptor ICOS, which are both critical for their differentiation and maintenance. In addition, T_{FH} cells secrete large amounts of IL-21, which aids in GC formation, isotype switching and plasma cell formation. In humans and mice, functionally similar T_{FH} cells can be found in secondary lymphoid organs. CXCR5+T_{FH} cells are also present in peripheral blood and seen at elevated levels in individuals with autoantibodies.

### B Cells

In some embodiments, the antigen-specific response is a B cell response. A B cell response can include secretion of antibodies. In some embodiments, the B cell response is an IgA, IgG, IgM, or IgE producing plasma cell response.

In certain embodiments, a B cell response after administration of a modified bacterium, as described herein, is an increase in antibody production by B cells. In some embodiments, the B cell response comprises an IgA, IgG, IgM, or IgE producing plasma cell response. In some embodiments, the B cell response comprises an IgA, IgG, IgM, or IgE producing memory B cell response. In some embodiments, the B cell response comprises increased production of IgA, IgG, IgM, or IgE antibodies by plasma cells and/or memory B cells. In certain embodiments, increased secretion of IgA, IgG, IgM, or IgE antibodies includes, but is not limited to, an increase in the number of B cells secreting IgA, IgG, IgM, or IgE antibodies as compared to the administration of a non-modified bacterium; an increase in the amount or volume of secreted IgA, IgG, IgM, or IgE antibodies as compared to the administration of a non-modified bacterium; enhanced secretion of IgA, IgG, IgM, or IgE antibodies by plasma cells or memory B cells as compared to the administration of a non-modified bacterium; and/or an induction of the secretion of IgA, IgG, IgM, or IgE antibodies by plasma cells or memory B cells as compared to the administration of a non-modified bacterium.

B cells are a part of the humoral immunity component of the adaptive immune system and secrete antibodies. B cells can also act as APCs and secrete cytokines. Immature B cells travel from the bone marrow to secondary lymphoid organs such as the spleen and lymph nodes. B cells are activated in the secondary lymphoid organs when they bind an antigen via the B cell receptor (BCR). There are multiple types of B cells, including plasmablasts, plasma cells, lymphoplasmacytoid cells, memory B cells, follicular (FO) B cells, marginal zone (MZ) B cells, B1 B cells, and regulatory B cells. FO B cells preferentially undergo T cell dependent activation. MZ B cells can undergo both T cell dependent and T cell independent activation. Once activated, B cells undergo a two-step differentiation process resulting in both short lived plasmablasts as well as long lived plasma cells and memory B cells. Plasma cells are long lived, non-proliferating cells that secrete antibodies that recognize a specific antigen. Memory B cells are a dormant B cell that function to provide a stronger, more rapid antibody response after a second encounter with an antigen or infection. T_{FH} cells are involved in the activation and differentiation of memory B cells. B cell differentiation, memory B cells, and antibody secretion by B cells are generally described in “Dynamics of B cells in germinal centres,” Nilushi S. et al., doi:10.1038/nri3804, Nature Reviews Immunology, 15, 137-148 (2015); “Memory B cells,” Tomohiro Kurosaki, Kohei Kometani & Wataru Ise, doi:10.1038/nri3802, Nature Reviews Immunology, 15, 149-159 (2015); and “The generation of antibody-secreting plasma cells,” Stephen L. Nutt, Philip D. Hodgkin, David M. Tarlinton & Lynn M. Corcoran, doi:10.1038/nri3795, Nature Reviews Immunology, 15, 160-171 (2015).

Exemplary B-cell surface markers include the B cell receptor (BCR), CD10, CD19, CD20 (MS4A1), CD21, CD22, CD23, CD24, CD37, CD40, CD53, CD72, CD73, CD74, CDw75, CDw76, CD77, CDw78, CD79a, CD79b, CD8β, CD81, CD82, CD83, CDw84, CD85, and CD86 leukocyte surface markers (for descriptions, see The Leukocyte Antigen Facts Book, 2^{nd} Edition. 1997, ed. Barclay et al. Academic Press, Harcourt Brace & Co., New York). Other B-cell surface markers include RP105, FcRH2, B-cell CR2, CCR6, P2X5, HLA-DOB, CXCR5, FCER2, BR3, Btig, NAG14, SLGC16270, FcRH1, IRTA2, ATWD578, FcRH3, IRTA1, FcRH6, BCMA, and 239287.

In some embodiments, the B cell response is an IgA, IgG, IgM, or IgE producing plasma cell response.

### Antigen Presenting Cells

In some embodiments, a modified microorganism expressing or displaying a non-native protein or peptide of interest is contacted with an APC, wherein the APC phagocytizes the modified microorganism and processes the heterologous antigen, or antigenic fragment thereof, for presentation on MHC-I or MHC-II molecules. In some embodiments, a modified microorganism is a live, recombinant commensal bacterium expressing or displaying a non-native protein or peptide of interest that is contacted with an APC, wherein the APC phagocytizes the recombinant bacterium and processes the heterologous antigen, or antigenic fragment thereof, for presentation on MHC-I or MHC-II molecules. In certain embodiments, examples of APCs include dendritic cells, macrophages, Langerhans cells, B cells, intestinal epithelial cells, and innate lymphoid cells, splenic dendritic cells, CD8+ dendritic cells, CD11b+ dendritic cells, plasmacytoid dendritic cells, follicular dendritic cells, monocytic cells, macrophages, bone marrow-derived macrophages, and Kupffer cells. In some embodiments, the APC is a dendritic cell, a splenic dendritic cell, a CD8+ dendritic cell, a CD11b+ dendritic cell, a plasmacytoid dendritic cell, a follicular dendritic cell, a monocytic cell, a macrophage, a bone marrow-derived macrophage, a Kupffer cell, a B-cell, a Langerhans cell, an innate lymphoid cell, a microglial cell, or an intestinal epithelial cell. In some embodiments, the APC is a dendritic cell, such as a CD103+CD11b+ dendritic cell. In some embodiments, the APC is an intestinal macrophage, such as a CX3CR1+ intestinal macrophage.

In some embodiments, the APC displaying the processed heterologous antigen in complex with an MHC molecule on its cell surface is then contacted with a T cell, such as a naïve T cell. In some embodiments, binding of the processed heterologous antigen/MHC complex to the T Cell Receptor (TCR) on the naïve T cell results in activation of the TCR and differentiation of the naïve T cell into a T_{reg}. In some embodiments, binding of the processed heterologous antigen/MHC complex to the T Cell Receptor (TCR) on the naïve T cell results in differentiation of the naïve T cell into an effector T cell (T_{eff}).

In certain embodiments, the induction of an antigen-specific T cell response can be detected using a suitable assay, such as cell surface marker expression analysis (e.g., by flow cytometry analysis) for specific T cell sub-populations. In certain embodiments, suitable assays for detecting T_{reg} and T_{H}2 cells are described herein or known by one of skill in the art.

In certain embodiments, in an in vitro method of inducing an antigen-specific T cell response, modified microorganisms expressing or displaying a heterologous antigen of interest are cultured with APCs in a suitable media under conditions that permit the APC to phagocytize the bacteria, process the heterologous antigen, and display the processed antigen on the cell surface. In certain embodiments, in an in vitro method of inducing an antigen-specific T cell response, live, recombinant commensal bacteria expressing or displaying a heterologous antigen of interest are cultured with APCs in a suitable media under conditions that permit the APC to phagocytize the bacteria, process the heterologous antigen, and display the processed antigen on the cell surface. In certain embodiments, naïve T cells can be added to the in vitro culture of APCs and bacteria, or the APCs can be isolated from the bacteria and cultured with the naïve T cells. In certain embodiments, the media can contain growth factors and cytokines that promote survival and differentiation of the T cells into a given T cell subset. In some embodiments, the media contains factors that promote the differentiation of T_{reg} cells, such as TGF-β. In some embodiments, the media contains factors that promote the differentiation of T_{eff} cells, such as IL-12, IL-2, and IFNγ.

In some embodiments, the T cells are primary T cells. In some embodiments, the T cells are primary T cells isolated from the gut or spleen of a subject. In some embodiments, the isolated T cells include fully differentiated T_{regs}. In some embodiments, freshly isolated primary T cells are cultured in basic medium (i.e., Dulbecco's Modified Eagle's Medium +5% Fetal Bovine Serum) without growth factors or cytokines.

In another embodiment of inducing an antigen-specific T cell response, the method is an in vivo method. In some embodiments, a subject is administered a pharmaceutical composition comprising a modified microorganism expressing or displaying a heterologous antigen of interest. In some embodiments, a subject is administered a pharmaceutical composition comprising a modified microorganism that is a live, recombinant commensal bacteria expressing or displaying a heterologous antigen of interest. The pharmaceutical composition can be administered by any suitable route, further described herein. In some embodiments the pharmaceutical composition is ingested by the subject for delivery of the recombinant bacteria to a native gastrointestinal niche in the subject. In some embodiments, the pharmaceutical composition is administered topically for delivery of the recombinant bacteria to an epidermal niche on the subject. In certain embodiments, upon administration of the pharmaceutical composition comprising a modified microorganism, the modified microorganism is phagocytized by an APC in the subject, processed, and presented to naïve T cells in the subject, thereby inducing an antigen-specific T cell response. In certain embodiments, upon administration of the pharmaceutical composition comprising a live, recombinant commensal bacteria, the live, recombinant commensal bacteria is phagocytized by an APC in the subject, processed, and presented to naïve T cells in the subject, thereby inducing an antigen-specific T cell response. In some embodiments, administration of the pharmaceutical composition elicits an antigen-specific T_{reg} response. In some embodiments, administration of the pharmaceutical composition elicits a T_{eff} response.

In some embodiments, differentiation into T_{reg}s is influenced by the type of bacteria engulfed by an APC. In some embodiments, a heterologous antigen can induce the differentiation of different T cell populations depending on the bacterial strain the heterologous antigen is expressed in. In some embodiments, a live, recombinant commensal bacterium derived from a bacterial strain that is commensal to a mammalian gut niche can induce a T_{reg} response specific for the heterologous antigen expressed by the recombinant bacterium, whereas the same heterologous antigen when expressed in a live, recombinant commensal bacterium derived from a bacterial strain that is commensal to a skin niche of a mammal induces the generation of an antigen-specific CD8+T_{eff} response.

In some embodiments, the bacterium induces a cytokine response comprising an increased expression of at least one of IL-10, IL-17A, IFNγ, IL-17F, IL-4, IL-5, IL-13, IL-21, or IL-22. In some embodiments, the bacterium induces a cytokine response comprising an increased expression of at least two, three, four, five, six, seven, or more of IL-10, IL-17A, IFNγ, IL-17F, IL-4, IL-5, IL-13, IL-21, or IL-22.

### 11. Methods for Detecting a T Cell or B Cell Response

In certain embodiments, an antigen-specific T cell or B cell response to the heterologous antigen can be detected by a variety of techniques known in the art. In certain embodiments, the T cell or B cell response can be detected by isolating lymphocytes from a subject administered with a live, recombinant commensal bacterium disclosed herein, or a pharmaceutical composition comprising the same, and assaying the lymphocytes ex vivo for the presence of antigen-specific T cells or B cells. Methods for detecting antigen-specific T cells isolated from human subjects are described, for example, in the “Manual of Molecular and Clinical Laboratory Immunology, 7^{th} Edition,” Editors: B. Detrick, R. G. Hamilton, and J. D. Folds, 2006, e-ISBN: 9781555815905. Methods for detecting antigen-specific B cells isolated from human subjects are described, for example, in “Techniques to Study Antigen-Specific B Cell Responses,” Jim Boonyaratanakornkit and Justin J. Taylor, Front. Immunol., 24 Jul. 2019, doi.org/10.3389/fimmu.2019.01694.

In certain embodiments, methods for detecting a T cell response to antigens include flow cytometry, cytokine assays (e.g. ELISA) and TCR sequencing. Flow cytometry can be used to detect expression of cell surface and/or intracellular markers before and after differentiation of a naïve T cell into an activated T cell. In certain embodiments, to detect an antigen-specific T_{reg} response, the cells can be labeled with antibodies that bind CD3, CD4, CD25, FOXP3, and CD127, and gated on cells that are CD3+, CD4+, CD25hi, FOXP3+, and CD127lo. In certain embodiments, because activated T cells often up-regulate CD25, and Foxp3 is expressed by effector (non-suppressive) T cell lineages, another gating strategy is to omit Foxp3 and sort cells that are CD3+, CD4+, CD25hi, and CD127lo cells. In certain embodiments, the population of sorted cells can then be assayed for T_{reg} properties, for example, by cytokine analysis and/or suppression co-culture assays with non-T_{reg} T cells (CD3+CD4+CD25−, CD127hi). In certain embodiments, inducible T_{regs} can also be detected by analyzing for expression of both RORγt and Foxp3 (see Xu M. et al., “c-Maf-dependent regulatory T cells mediate immunological tolerance to a gut pathobiont,” Nature. 2018 Feb. 15; 554(7692): 373-377).

In certain embodiments, other assays to detect antigen-specific T_{reg} cells include suppression assays. In certain embodiments, responder CD4+ T cells are stimulated polyclonally and co-cultured with different ratios of putative T_{reg} cells, and the cultures are treated with ³H-thymidine to monitor DNA synthesis of responder T cells. In certain embodiments, T_{reg} cells can also be detected by measuring the production of IL-2 and IFN-7 in the coculture assays, as the level of these cytokines is decreased by T_{reg} suppression of responder T cells. In certain embodiments, another assay to detect an antigen-specific T_{reg} response is to detect the expression of IL-2 and IFN-7 mRNA or CD69 and CD154 surface protein expression in responder T cells, where suppression can be detected within 5-7 hours of coculturing the responder T cells with putative T_{reg} cells. See McMurchy et al., “Suppression assays with human T regulatory cells: A technical guide,” Eur. J. Immunol. 2012. 42: 27-34, which is incorporated by reference herein.

In certain embodiments, additional assays to detect an antigen-specific T_{reg} response include sequence analysis of single cell mRNA as described in Miragaia et al., “Single-Cell Transcriptomics of Regulatory T Cells Reveals Trajectories of Tissue Adaptation,” Immunity 50, 493-504, Feb. 19, 2019; and transcriptome profiling as described in Bhairavabhotla et al., Transcriptome Profiling of Human FoxP3+ Regulatory T Cells,” Human Immunology, Volume 77, Issue 2, February 2016, Pages 201-213. In certain embodiments, another assay for detecting an antigen-specific T_{reg} response comprises sequencing the TCR of T_{reg} cells, as described in Rossetti et al., “TCR repertoire sequencing identifies synovial T_{reg} cell clonotypes in the bloodstream during active inflammation in human arthritis,” Ann Rheum Dis 2017; 76:435-441 (doi:10.1136/annrheumdis-2015-208992).

In certain embodiments, another assay for detecting an antigen-specific T_{reg} response involves detecting DNA methylation of the FoxP3 locus in T cells, as described in Baron U. et al., “DNA demethylation in the human FOXP3 locus discriminates regulatory T cells from activated FOXP3(+) conventional T cells,” Eur J Immunol 2007; 37:2378-89 (doi:10.1002/eji.200737594).

In some embodiments, the assay for detecting an antigen-specific T_{reg} response uses an APC, heterologous antigen (or heterologous antigen-expressing or -displaying bacteria) and T cell co-culture system. In certain embodiments, after a suitable period of co-culture (e.g., about 1, 2, 3, 4, or 5 hours of co-culture), expression of Nur77 is monitored to detect antigen-specific TCR activation.

In certain embodiments, to detect an antigen-specific T_{eff} response, cells can be labeled with antibodies that bind to T cell markers that are characteristic of specific T cell lineages and the proportion of different T cell subset populations can be analyzed using techniques known by persons of skill in the art (e.g., see Syrbe, et al. (1999) Springer Semin Immunopathol 21, 263-285; Luckheeram R V et al. (2012). Clin Dev Immunol. 2012; 2012:925135; and Mahnke Y D et al. (2013) Cytometry A 83(5):439-440). In some embodiments, cells can be labelled with one or more antibodies that bind CD3, CD8, CCR7, IFNγ, T-bet, CXCR3, CCR5, IL-4, IL-5, GATA3, STAT6, CCR4, CCR8, IL-17, RORγT, or CCR6. In a further example, to identify CD8+ T cells, cells can be labeled with antibodies that bind CD3, CD8, and CCR7 and gated on cells that are CD3+, CD8+, and CCR7−.

In certain embodiments, assays for detecting an antigen-specific T_{eff} response are well known by persons of skill in the art. In some embodiments, the assay for detecting an antigen-specific T_{eff} response uses an APC, heterologous antigen (or heterologous antigen-expressing or -displaying bacteria) and T cell co-culture system. After a suitable period of co-culture (e.g., about 1, 2, 3, 4, or 5 hours of co-culture), expression of Nur77 is monitored to detect antigen-specific TCR activation (e.g., see Ashouri J F and Weiss A (2017) J Immunol. 198 (2) 657-668).

In certain embodiments, other assays to detect antigen-specific T_{eff} cells include proliferation assays. In certain embodiments, responder CD8+ T cells are stimulated polyclonally and co-cultured with different ratios of putative T_{eff} cells, and the cultures are treated with ³H-thymidine to monitor DNA synthesis of responder T cells. In certain embodiments, T_{eff} cells can also be detected by measuring the production of cytokines (e.g., IFN-γ) in coculture assays, as well as measuring the production of perforin and granzyme.

In certain embodiments, assays for detecting an antigen-specific B cell response are well known by persons of skill in the art. In certain embodiments, such assays include flow cytometry, ELISPOT, RNA-seq, DNA barcoding, limiting dilution, and mass cytometry. In certain embodiments, methods for detecting a B cell response to antigens include flow cytometry, ELISPOT and BCR sequencing. Flow cytometry can be used to detect expression of cell surface B cell receptor (BCR) and other B cell surface markers.

### 12. Methods of Treatment

Also provided are methods of preventing or treating a disease, disorder or condition in a subject with a pharmaceutical composition comprising a recombinant bacterium, or surface-labeled bacterium, described herein. In some embodiments, the disease, disorder or condition in a subject is an autoimmune disease, disorder or condition in a subject. In some embodiments, the disease, disorder or condition in a subject is an infectious disease. In some embodiments, the disease, disorder or condition in a subject is a cancer or proliferative disorder. In some embodiments, the administration of the bacterium or pharmaceutical composition comprising a recombinant bacterium or surface-labeled bacterium described herein induces a T cell or B cell response. In some embodiments, the administration of the bacterium or pharmaceutical composition comprising a recombinant bacterium or surface-labeled bacterium described herein induces a T_{eff} T cell response. In some embodiments, the administration of the bacterium or pharmaceutical composition comprising a recombinant bacterium or surface-labeled bacterium described herein induces a T_{reg} T cell response. In some embodiments, the administration of the bacterium or pharmaceutical composition comprising a recombinant bacterium or surface-labeled bacterium described herein induces a T_{H}2 T cell response. In some embodiments, the administration of the bacterium or pharmaceutical composition comprising a recombinant bacterium or surface-labeled bacterium described herein induces an immune response. In some embodiments, the immune response promotes differentiation of T_{H}2 T cells in the host. In some embodiments, the immune response limits differentiation of T_{H}1 T cells in the host.

In some embodiments, the method comprises administering a therapeutically effective amount of a pharmaceutical composition comprising a modified microorganism, e.g., a live recombinant commensal bacterial cell or strain, described herein to the subject. In certain embodiments, the pharmaceutical composition can be administered to the subject by any suitable route that does not trigger an adverse reaction in the subject. In certain embodiments, the pharmaceutical composition can be administered by oral, nasal, vaginal, rectal, topical, subcutaneous, intradermal or intramuscular routes. In some embodiments, the pharmaceutical composition is ingested orally by the subject, administered topically to the subject, inhaled by the subject, or injected into the subject. In some embodiments, the pharmaceutical composition is administered in a material, such as a delayed release enteric coating, that permits transit through the stomach to the small intestine before the pharmaceutical is released. In some embodiments, the pharmaceutical composition comprises a enteric-coated capsule containing a modified microorganism, e.g., a live, recombinant commensal bacterium described herein.

In some embodiments, pharmaceutical compositions comprising a modified microorganism, e.g., a live recombinant commensal bacterium, described herein, is used for the prevention or treatment of an autoimmune disease. In certain embodiments, examples of autoimmune diseases that can be treated by a modified microorganism disclosed herein include multiple sclerosis, psoriasis, celiac disease, diabetes mellitus Type I, rheumatoid arthritis, systemic lupus erythematosus, inflammatory bowel disease, Graves' disease, Hashimoto's autoimmune thyroiditis, vitiligo, rheumatic fever, pernicious anemia/atrophic gastritis, alopecia areata, immune thrombocytopenic purpura, temporal arteritis, ulcerative colitis, Crohn's disease, scleroderma, antiphospholipid syndrome, autoimmune hepatitis type 1, primary biliary cirrhosis, Sjogren's syndrome, Addison's disease, dermatitis herpetiformis, Kawasaki disease, sympathetic ophthalmia, HLA-B27 associated acute anterior uveitis, primary sclerosing cholangitis, discoid lupus erythematosus, polyarteritis nodosa, CREST Syndrome, myasthenia gravis, polymyositis/dermatomyositis, Still's disease, autoimmune hepatitis type 2, Wegener's granulomatosis, mixed Connective tissue disease, microscopic polyangiitis, autoimmune polyglandular syndrome, Felty's syndrome, autoimmune hemolytic anemia, chronic inflammatory demyelinating polyneuropathy, Guillain-Barre Syndrome, Behcet disease, autoimmune neutropenia, bullous pemphigoid, essential mixed cryoglobulinemia, linear morphea, autoimmune polyglandular syndrome 1 (APECED), acquired hemophilia A, Batten disease/neuronal ceroid lipofuscinoses, autoimmune pancreatitis, Hashimoto's encephalopathy, Goodpasture's disease, pemphigus vulgaris, autoimmune disseminated encephalomyelitis, relapsing polychondritis, Takayasu arteritis, Churg-Strauss syndrome, epidermolysis bullosa acquisita, cicatricial pemphigoid, pemphigus foliaceus, autoimmune hypoparathyroidism, autoimmune hypophysitis, autoimmune inner ear disease, autoimmune lymphoproliferative syndrome, autoimmune oophoritis, autoimmune orchitis, autoimmune polyglandular syndrome, Cogan's syndrome, encephalitis lethartica, erythema elevatum diutinum, Evans syndrome, immunodysregulation polyendocrinopathy enteropathy X-linked (IPEX), Issac's syndrome/acquired neuromyotonia, Miller Fisher syndrome, Morvan's syndrome, PANDAS, POEMS syndrome, Rasmussen's encephalitis, stiff-person syndrome, Vogt-Koyanagi-Harada syndrome, neuromyelitis optica, graft vs host disease, esophageal encephalitis, and autoimmune uveitis.

In some embodiments, pharmaceutical compositions comprising a modified microorganism, e.g., a live recombinant commensal bacterium, described herein, is used for the prevention or treatment of a proliferative disorder. In some embodiments, the proliferative disorder is cancer. In some embodiments, the cancer is melanoma, kidney, hepatobiliary, head-neck squamous carcinoma (HNSC), pancreatic, colon, bladder, glioblastoma, prostate, lung, breast (mammary), ovarian, gastric, kidney, bladder, esophageal, renal, melanoma, leukemia, lymphoma, mesothelioma, basal cell carcinoma, squamous cell carcinoma, or testicular cancer.

In some embodiments, pharmaceutical compositions comprising a modified microorganism, e.g., a live recombinant commensal bacterium, described herein, is used for the prevention or treatment of a proliferative disease. In certain embodiments, examples of proliferative diseases include melanoma, basal cell carcinoma, squamous cell carcinoma, and testicular cancer.

In some embodiments, pharmaceutical compositions comprise a modified microorganism, e.g., a live recombinant commensal bacterium described herein, engineered to express or surface-labeled to display a neoantigen or tumor-associated antigen identified in cancer cells from an individual cancer subject. In certain embodiments, a live, recombinant commensal bacterium engineered to express or surface-labeled to display an identified neoantigen or tumor-associated antigen, can be administered in a pharmaceutical formulation to elicit an adaptive T cell response in the cancer subject or ex vivo cultured with HLA-matched donor T cells that can subsequently be introduced into the cancer subject to recognize and kill the cancer cells.

Any suitable animal model can be used to test the methods described herein. In some embodiments, the animal model is a mouse model, or a non-human primate model.

In some embodiments, pharmaceutical compositions comprising a modified microorganism, e.g., a live recombinant commensal bacterium described herein, is used for the prevention or treatment of a proliferative disease. In certain embodiments, examples of proliferative diseases include melanoma, basal cell carcinoma, squamous cell carcinoma, and testicular cancer.

Any suitable animal model can be used to test the methods described herein. In some embodiments, the animal model is a mouse model, or a non-human primate model.

In some embodiments, a recombinant commensal bacterium is co-administered with one or more additional agents. In certain embodiments, a therapeutically effective amount of one or more additional agents can be co-administered. In certain embodiments, co-administration generally refers to administering two or more agents (e.g., a recombinant commensal bacterium and a second agent), such that each agent is capable of exerting their pharmacological effect during the same period of time; such co-administration can be achieved by either simultaneous, contemporaneous, or sequential administration of the two or more agents. In certain embodiments, agents that can be co-administered include immune checkpoint inhibitors, chemotherapeutic agents, and/or cell-based therapies. In certain embodiments, illustrative immune checkpoint inhibitors include, but are not limited to, Tremelimumab (CTLA-4 blocking antibody), anti-OX40, PD-L1 monoclonal antibody (Anti-B7-H1; MEDI4736), ipilimumab, MK-3475 (PD-1 blocker), Nivolumamb (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). In certain embodiments, illustrative chemotherapeutic agents include, but are not limited to, alkylating agents such as cyclophosphamide, mechlorethamine, chlorambucil, melphalan, dacarbazine (DTIC), nitrosoureas, temozolomide (oral dacarbazine); anthracyclines, such as daunorubicin, doxorubicin, epirubicin, idarubicin, mitoxantrone, and valrubicin; cytoskeletal disruptors, such as paclitaxel, nab-paclitaxel, docetaxel, abraxane, and taxotere; epothilones; histone deacetylase inhibitors such as vorinostat and romidepsin; inhibitors of topoisomerase I such as irinotecan and topotecan; inhibitors of topoisomerase II such as etoposide, teniposide and tafluposide; kinase inhibitors such as bortezomib, erlotinib, gefitinib, imatinib, vemurafenib and vismodegib; nucleotide analogs and precursor analogs such as azacitidine, azathioprine, capecitabine; peptide antibiotics such as bleomycin and actinomycin; platinum-based agents, such as carboplatin, cisplatin and oxaliplatin; retinoids such as tretinoin and alitretinoin; and vinca alkaloids and derivatives such as vinblastine, vincristine, vindesine and vinorelbine. In certain embodiments, cell-based therapies include, but are not limited to, immune cells engineered to express chimeric antigen receptors (e.g., CAR-T and/or CAR-NK therapies) or exogenous T cell receptors.

### 13. Kits Comprising the Bacterial Strains

In another aspect, a kit comprising the modified microorganism, e.g., the live recombinant commensal bacterium is provided. In certain embodiments, khe kit can include a live, recombinant commensal bacterium that expresses a heterologous antigen described herein. In some embodiments, the heterologous antigen is an antigen normally present in a non-bacterial host of the commensal bacterium. In certain embodiments, the heterologous antigen can be an antigen that is expressed by or present in a vertebrate or mammal.

In some embodiments, a kit comprises a pharmaceutical composition described herein. In certain embodiments, the kit can include a pharmaceutical composition comprising a modified microorganism, e.g., a live, recombinant commensal bacterium that expresses a heterologous antigen. In some embodiments, the pharmaceutical composition is capable of inducing a regulatory T cell response to the heterologous antigen. In some embodiments, the pharmaceutical composition is capable of inducing an effector T cell response to the heterologous antigen.

In some embodiments, the kit can also include instructions for administering the pharmaceutical composition to a subject. In certain embodiments, the kit can include pharmaceutical excipients that aid in administering the pharmaceutical compositions.

In some embodiments, the kit can also include additional agents that are useful for treating a disease or pathological condition in a subject. In certain embodiments, examples of additional agents include small molecule drugs or antibodies that are useful for treating a disease or pathological condition in a subject.

### Further Non-Limiting Embodiments

Additional non-limiting embodiments of the disclosure are described in the following aspects:
1. A live, recombinant commensal bacterium, wherein the bacterium is engineered to express a fusion protein comprising (a) a non-native protein or peptide and (b) a tat signal sequence peptide, a sec signal sequence peptide, or a sortase-derived signal sequence peptide, wherein the non-native protein or peptide is associated with a host disease or condition, wherein upon administration of the bacterium to the host resulting in colonization of a native host niche by the bacterium, the host mounts an adaptive immune response to the non-native protein or peptide, and wherein the adaptive immune response is a T-cell response.
2. A live, recombinant bacterium, wherein the bacterium is engineered to express a fusion protein comprising (a) a non-native protein or peptide and (b) an antigen-presenting cell (APC) targeting moiety.
3. The recombinant commensal bacterium of aspect 2, wherein the non-native protein or peptide is associated with a host disease or condition, wherein upon administration of the bacterium to the host resulting in colonization of a native host niche by the bacterium, the host mounts an adaptive immune response to the non-native protein or peptide.
4. The recombinant commensal bacterium of aspect 3, wherein the adaptive immune response is a T-cell response or a B cell response.
5. A live, recombinant commensal bacterium, wherein the bacterium is engineered to express a fusion protein comprising a non-native protein or peptide, wherein the non-native protein or peptide is associated with a host disease or condition, wherein upon administration of the bacterium to the host resulting in colonization of a native host niche by the bacterium, the host mounts an adaptive immune response to the non-native protein or peptide, and wherein the commensal bacterium is selected from the group consisting of: Corynebacterium tuberculostearicum, Corynebacterium accolens, Corynebacterium amycolatum, Corynebacterium aurimucosum, Corynebacterium propinquum, Corynebacterium pseudodiphtheriticum, Corynebacterium granulosum, Cutibacterium acnes, Cutibacterium avidum, Dolosigranulum pigrum, Finegoldia magna, Moraxella catarrhalis, Moraxella nonliquefaciens, Haemophilus influenzae, Haemophilus aegyptius, Rothia mucilaginosa, Streptococcus pyogenes, Streptococcus agalactiae, Streptococcus gordonii, Neisseria lactamica, Neisseria cinerea, Neisseria mucosa, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium breve ATCC 15700, Bifidobacterium longum, Veillonella parvula, Gardnerella vaginalis, Atopobium vaginae, Prevotella bivia, Mobiluncus mulieris, Mageeibacillus indolicus, Prevotella buccalis, Enterococcus faecium, Lactococcus lactis, Ruminococcus gnavus JCM6515, and Eubacterium limosum ATCC 8486.
6. The recombinant commensal bacterium of aspect 5, wherein the commensal bacterium is selected from the group consisting of: a bacterium having ATCC accession number 35692, 49725, 49726, 49368, 700975, 700540, 51488, 10700, 25564, 51277, 11827, 25577, 49753, 51524, 29328, 25238, 25240, 19976, 51907, 11116, 25296, 19615, 12344, BAA-611, 13813, 10558, 23970, 14685, 19696, 33820, 25258, 19992, 55195, 4356, 33200, 7469, 393, 7995D-5, 23272, 11741, 15700, 15697, 10790, 17745, 14018, BAA-55, 29303, 35243, BAA-2120, 35310, 19434, 19435, 29149, and 8486.
7. The recombinant commensal bacterium of aspect 5, wherein the commensal bacterium is selected from the group consisting of: Lactobacillus casei, Lactococcus lactis, Streptococcus gordonii, Lactobacillus crispatus, Lactobacillus iners, Cutibacterium acnes, Streptococcus agalactiae, Ruminococcus gnavus JCM6515, Neisseria lactamica, Bifidobacterium breve ATCC 15700, and Bifidobacterium longum.
8. The recombinant commensal bacterium of aspect 7, wherein the commensal bacterium is selected from the group consisting of: a bacterium having ATCC accession number 393, 19435, 35105, 33820, 55195, 6919, 13813, 23970, 15700, and 15707, and a bacterium having an accession number JCM6515.
9. The recombinant commensal bacterium of any one of aspects 5-8, wherein the adaptive immune response is a T cell response or a B cell response.
10. A live, recombinant commensal bacterium, wherein the bacterium is engineered to express (a) a first non-native protein or peptide, wherein the first non-native protein or peptide is engineered to elicit a CD4+ T cell response, and (b) a second non-native protein or peptide, wherein the second non-native protein or peptide is engineered to elicit a CD8+ cytotoxic T cell response.
11. A composition comprising: (a) a first recombinant commensal bacterium engineered to express a first non-native protein or peptide, wherein the first non-native protein or peptide is engineered to elicit a CD4+ T cell response, and (b) a second recombinant commensal bacterium engineered to express a non-native protein or peptide, wherein the second non-native protein or peptide is engineered to elicit a CD8+ cytotoxic T cell response.
12. The recombinant commensal bacterium of aspect 10 or the composition of aspect 11, wherein the first non-native protein or peptide and the second non-native protein or peptide are each derived from a shared antigen.
13. The composition of aspect 12, wherein the first non-native protein or peptide and the second non-native protein or peptide derived from the shared antigen comprise different amino acid sequences.
14. The recombinant commensal bacterium of aspect 10 or the composition of aspect 11, wherein the first non-native protein or peptide and the second non-native protein or peptide are each derived from a different antigen.
15. A live, recombinant commensal bacterium, wherein the bacterium is engineered to express a fusion protein comprising a non-native protein or peptide, wherein the non-native protein or peptide is associated with an infection, wherein upon administration of the bacterium to the host resulting in colonization of a native host niche by the bacterium, the host mounts an adaptive immune response to the non-native protein or peptide.
16. The recombinant commensal bacterium of aspect 15, wherein the adaptive immune response is a T-cell response or a B cell response.
17. The recombinant commensal bacterium of any one of aspects 1-16, wherein the adaptive immune response is distal from the site of administration.
18. The recombinant commensal bacterium of any one of aspects 1-17, wherein the adaptive immune response is distal from the native host niche.
19. The recombinant commensal bacterium of aspects 17 or 18, wherein the distal adaptive immune response comprises an immune response in an organ that is not the organ of the site of administration and/or the native host niche.
20. The recombinant commensal bacterium of any one of aspects 17-19, wherein the site of administration and/or the native host niche comprises skin.
21. The recombinant commensal bacterium of any one of aspects 17-20, wherein the distal adaptive immune response comprises an antitumor response.
22. The recombinant commensal bacterium of aspect 21, wherein the antitumor response targets a metastasis.
23. The recombinant commensal bacterium of any one of aspects 1-22, wherein the colonization of the native host niche is persistent or transient.
24. The recombinant commensal bacterium of any one of aspects 1-23, wherein the native host niche is persistently colonized, and wherein colonization is for at least 60 days, at least 112 days, at least 178 days, at least 1 year, at least 2 years, or at least 5 years.
25. The recombinant commensal bacterium of any one of aspects 1-23, wherein the native host niche is persistently colonized, and wherein colonization is for at least 180 days.
26. The recombinant commensal bacterium of aspect 24 or 25, wherein the persistent colonization provides a persistent antigen source, optionally wherein the antigen stimulates an antigen-specific T cell population and produces a persistent antigen-specific T cell population.
27. The recombinant commensal bacterium of any one of aspects 1-23, wherein the native host niche is transiently colonized, and wherein colonization is for 1 day to 60 days.
28. The recombinant commensal bacterium of any one of aspects 1-23, wherein the native host niche is transiently colonized, and wherein colonization is for 3.5 days to 60 days.
29. The recombinant commensal bacterium of aspect 27 or 28, wherein the native host niche is transiently colonized, and wherein colonization is for 7 days to 28 days.
30. The recombinant commensal bacterium of any one of aspects 1-29, wherein colonization is determined by polymerase chain reaction or colony forming assay performed on a sample obtained from the host after 1 day, 3.5 days, 7 days, 14 days, 28 days, or 60 days after administration to the host.
31. The recombinant commensal bacterium of any one of aspects 1-30, wherein the administration results in interaction of the bacterium with a native immune system partner cell.
32. The recombinant commensal bacterium of aspect 31, wherein the native immune system partner cell is an antigen-presenting cell.
33. The recombinant commensal bacterium of aspect 32, wherein the antigen-presenting cell is selected from the group consisting of a dendritic cell, a macrophage, a B-cell, and an intestinal epithelial cell.
34. The recombinant commensal bacterium of any one of aspects 1-33, wherein the native host niche is selected from the group consisting of the gastrointestinal tract, respiratory tract, urogenital tract, and skin.
35. The recombinant commensal bacterium of any one of aspects 1-34, wherein the non-native protein or peptide is a host protein or peptide.
36. The recombinant commensal bacterium of any one of aspects 1-35, wherein the bacterium is a Gram-negative bacterium.
37. The recombinant commensal bacterium of aspect 36, wherein the Gram-negative bacterium is selected from the group consisting of Bacteroides thetaiotaomicron, Helicobacter hepaticus and Parabacteroides sp.
38. The recombinant commensal bacterium of any one of aspects 1-35, wherein the bacterium is a Gram-positive bacterium.
39. The recombinant commensal bacterium of aspect 38, wherein the Gram-positive bacterium is selected from the group consisting of Staphylococcus epidermidis, Faecalibacterium sp., Corynebacterium spp., Eubacterium limosum, Ruminococcaceae bacterium cv2, Clostridium sp., Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus AGR2154, Butyrate-producing bacterium SSC/2, Clostridium sp. ASF356, Coprobacillus sp. D6 cont1.1, Eubacterium sp. 3_1_31 cont1.1, Erysipelotrichaceae bacterium 213, Ruminococcus bromii L2-63, Firmicutes bacterium ASF500, Firmicutes bacterium ASF500, Bifidobacterium animalis subsp. lactis ATCC 27673, and Bifidobacterium breve UCC2003. 40. The recombinant commensal bacterium of aspect 39, wherein the Gram-positive bacterium is selected from the group consisting of Staphylococcus epidermidis, Faecalibacterium sp., Corynebacterium spp., and Clostridium sp.
41. The recombinant commensal bacterium of aspect 40, wherein the bacterium is selected from the group consisting of Staphylococcus epidermidis and Corynebacterium spp.
42. The recombinant commensal bacterium of aspect 41, wherein the bacterium is S. epidermidis NIHLM087.
43. The recombinant commensal bacterium of any one of aspects 1-4 or 10-35, wherein the bacterium is selected from the group consisting of: Corynebacterium tuberculostearicum, Corynebacterium accolens, Corynebacterium amycolatum, Corynebacterium aurimucosum, Corynebacterium propinquum, Corynebacterium pseudodiphtheriticum, Corynebacterium granulosum, Cutibacterium acnes, Cutibacterium avidum, Dolosigranulum pigrum, Finegoldia magna, Moraxella catarrhalis, Moraxella nonliquefaciens, Haemophilus influenzae, Haemophilus aegyptius, Rothia mucilaginosa, Streptococcus pyogenes, Streptococcus agalactiae, Streptococcus gordonii, Neisseria lactamica, Neisseria cinerea, Neisseria mucosa, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium breve ATCC 15700, Bifidobacterium longum, Veillonella parvula, Gardnerella vaginalis, Atopobium vaginae, Prevotella bivia, Mobiluncus mulieris, Mageeibacillus indolicus, Prevotella buccalis, Enterococcus faecium, Lactococcus lactis, Ruminococcus gnavus JCM6515, and Eubacterium limosum.
44. The recombinant commensal bacterium of aspect 43, wherein the commensal bacterium is selected from the group consisting of: a bacterium having ATCC accession number 35692, 49725, 49726, 49368, 700975, 700540, 51488, 10700, 25564, 51277, 11827, 25577, 49753, 51524, 29328, 25238, 25240, 19976, 51907, 11116, 25296, 19615, 12344, BAA-611, 13813, 10558, 23970, 14685, 19696, 33820, 25258, 19992, 55195, 4356, 33200, 7469, 393, 7995D-5, 23272, 11741, 15700, 15697, 10790, 17745, 14018, BAA-55, 29303, 35243, BAA-2120, 35310, 19434, 19435, 29149, and 8486.
45. The recombinant commensal bacterium of aspect 43, wherein the commensal bacterium is selected from the group consisting of: Lactobacillus casei, Lactococcus lactis, Streptococcus gordonii, Lactobacillus crispatus, Lactobacillus iners, Cutibacterium acnes, Streptococcus agalactiae, Ruminococcus gnavus, Neisseria lactamica, Bifidobacterium breve, and Bifidobacterium longum.
46. The recombinant commensal bacterium of aspect 45, wherein the commensal bacterium is selected from the group consisting of: a bacterium having ATCC accession number 393, 19435, 35105, 33820, 55195, 6919, 13813, 23970, 15700, and 15707, and a bacterium having an accession number JCM6515.
47. The recombinant commensal bacterium of any one of aspects 1-46, wherein the administration is via a route selected from the group consisting of topical, enteral, and inhalation.
48. The recombinant commensal bacterium of aspect 47, wherein the route is topical.
49. The recombinant commensal bacterium of aspect 47, wherein the route is enteral.
50. The recombinant commensal bacterium of any one of aspects 1-14 or 23-49, wherein the protein or peptide is associated with an infection.
51. The recombinant commensal bacterium of aspect 50, wherein the infection is selected from the group consisting of a viral infection, a parasitic infection, a bacterial infection, or a fungal infection.
52. The recombinant commensal bacterium of aspect 50 or 51, wherein the infection occurs at or is otherwise associated with a mucosal boundary of the host.
53. The recombinant commensal bacterium of any one of aspects 50-52, wherein the non-native protein or peptide is derived from a virus, a parasite, a bacterium, or a fungus associated with the infection.
54. The recombinant commensal bacterial of aspect 53, wherein the non-native protein or peptide is derived from influenza, HSV, HIV, or SARS-Cov-2.
55. The recombinant commensal bacterial of aspect 54, wherein the non-native protein or peptide is selected from the group consisting of: NP366-374, NP306-322, NA177-193, M2 ectodomain, HA2 stem-HA 212-64, HA2 stem-HA 276-130, gB glycoprotein, gd glycoprotein, gB glycoprotein 498-505, SARS-Cov2 Spike protein, HIV-gp120, HIV-gp41, HIV V1V2 apex, HIV V3 loop, HIV CD4 binding site, gp120/gp41 interface, gp120 silent face, and HIV membrane-proximal external region (MPER).
56. The recombinant commensal bacterium of any one of aspects 1-49, wherein the protein or peptide is associated with an autoimmune disorder.
57. The recombinant commensal bacterium of any one of aspects 1-49, wherein the protein or peptide is associated with a proliferative disorder.
58. The recombinant commensal bacterium of aspect 57, wherein the proliferative disorder is cancer.
59. The recombinant commensal bacterium of aspect 58, wherein the cancer is selected from melanoma, basal cell carcinoma, squamous cell carcinoma, testicular cancer, sarcoma, and prostate cancer.
60. The recombinant commensal bacterium of aspect 58, wherein the cancer is melanoma.
61. The recombinant commensal bacterium of aspect 60, wherein the non-native protein or peptide is derived from a melanocyte-specific antigen selected from the group consisting of PMEL, TRP2 and MART-1.
62. The recombinant commensal bacterium of any one of aspects 57-60, wherein the non-native protein or peptide comprises a neoantigen, wherein the neoantigen comprises at least one mutation that makes the non-native protein or peptide distinct from a protein or peptide encoded by a wild-type gene of the host.
63. The recombinant commensal bacterium of aspect 62, wherein the neoantigen is selected from the group consisting of: Ints11, Kif18 bp, T3 sarcoma neoantigens, and a neoantigen expressed by the TRAMPC2 prostate cancer cell line.
64. The recombinant commensal bacterium of any one of aspects 2-63, wherein the fusion protein further comprises a signal sequence peptide.
65. The recombinant commensal bacterium of any one of aspects 1-64, wherein the signal sequence peptide directs tethering of the fusion protein to a cell wall of the bacterium following expression.
66. The recombinant commensal bacterium of aspect 65, wherein the signal sequence peptide that directs tethering comprises a sortase-derived signal sequence peptide, optionally wherein the sortase-derived signal sequence peptide comprises one or more sequences derived from Protein A of Staphylococcus aureus.
67. The recombinant commensal bacterium of aspect 65 or 66, wherein the signal sequence peptide that directs tethering is N-terminal of the non-native protein or peptide and the fusion protein comprises a cell-wall spanning peptide domain C-terminal of the non-native protein or peptide.
68. The recombinant commensal bacterium of any one of aspects 1-64, wherein the signal sequence peptide directs secretion of the fusion protein from the bacterium following expression.
69. The recombinant commensal bacterium of aspect 68, wherein the signal sequence peptide that directs secretion comprises a tat signal sequence peptide.
70. The recombinant commensal bacterium of aspect 69, wherein the tat signal sequence peptide comprises an S. aureus derived signal sequence peptide.
71. The recombinant commensal bacterium of aspect 70, wherein the S. aureus derived signal sequence peptide is derived from fepB.
72. The recombinant commensal bacterium of aspect 68, wherein the signal sequence peptide that directs secretion comprises a sec signal sequence peptide.
73. The recombinant commensal bacterium of aspect 72, wherein the sec signal sequence peptide comprises an S. epidermidis derived signal sequence peptide.
74. The recombinant commensal bacterium of aspect 73, wherein the S. epidermidis derived signal sequence peptide is derived from predicted sec-secreted S. epidermidis protein (gene locus HMPREF9993_06668).
75. The recombinant commensal bacterium of any one of aspects 1 or 5-74, wherein the fusion protein further comprises an antigen-presenting cell (APC) targeting moiety, optionally wherein the APC targeting moiety comprises a CD11b or a MHC II targeting moiety.
76. The recombinant commensal bacterium of aspect 75, wherein the APC targeting moiety comprises a nanobody (VHH) antibody binding domain, optionally wherein the VHH antibody binding domain comprises the sequence of SEQ ID NO:33 or SEQ ID NO:34.
77. The recombinant commensal bacterium of any one of aspects 1-76, wherein the bacterium is engineered to express a fusion protein comprising the protein or peptide and a native bacterial protein or portion thereof.
78. The recombinant commensal bacterium of aspect 77, wherein the protein or peptide is fused to the N-terminus or the C-terminus of the native bacterial protein or portion thereof.
79. The recombinant commensal bacterium of any one of aspects 1-78, wherein the bacterium is formulated for administration in combination with a high-complexity defined microbial community.
80. The recombinant commensal bacterium of any one of aspects 1-79, wherein the host is a mammal.
81. The recombinant commensal bacterium of aspect 80, wherein the mammal is a human.
82. A polynucleotide used to engineer the recombinant commensal bacterium of any one of aspects 1-81.
83. A method for generating an antigen-presenting cell displaying an antigen derived from a non-native protein or peptide, comprising: administering the recombinant commensal bacterium of any one of aspects 1-81 to a subject, wherein the administration results in colonization of the native host niche by the bacterium, internalization of the bacterium or the non-native protein or peptide by an antigen-presenting cell, and presentation of the antigen by the antigen-presenting cell.
84. The method of aspect 83, wherein the colonization of the native host niche is persistent or transient.
85. The method of aspect 84, wherein the native host niche is persistently colonized, and wherein colonization is for at least 60 days, at least 112 days, at least 178 days, at least 1 year, at least 2 years, or at least 5 years.
86. The method of aspect 84, wherein the native host niche is persistently colonized, and wherein colonization is for at least 180 days.
87. The method of aspect 85 or 86, wherein the persistent colonization provides a persistent antigen source, optionally wherein the antigen stimulates an antigen-specific T cell population and produces a persistent antigen-specific T cell population.
88. The method of aspect 84, wherein the native host niche is transiently colonized, and wherein colonization is for 1 day to 60 days.
89. The method of aspect 84, wherein the native host niche is transiently colonized, and wherein colonization is for 3.5 days to 60 days.
90. The method of aspect 88 or 89, wherein the native host niche is transiently colonized, and wherein colonization is for 7 days to 28 days.
91. The method of any one of aspects 83-90, wherein colonization is determined by polymerase chain reaction or colony forming assay performed on a sample obtained from the host after 1 day, 3.5 days, 7 days, 14 days, 28 days, or 60 days after administration to the host.
92. The method of any one of aspects 83-91, wherein the administration results in interaction of the bacterium with a native immune system partner cell.
93. The method of aspect 92, wherein the native immune system partner cell is the antigen-presenting cell.
94. The method of aspect 93, wherein the antigen-presenting cell is selected from the group consisting of a dendritic cell, a macrophage, a B-Cell, and an intestinal epithelial cell.
95. The method of any one of aspects 83-94, wherein the native host niche is selected from the group consisting of the gastrointestinal tract, respiratory tract, urogenital tract, and skin.
96. The method of any one of aspects 83-95, wherein the presentation is within an MHC II complex.
97. The method of any one of aspects 83-95, wherein the presentation is within an MHC I complex.
98. A method for generating a T cell response in a subject, comprising: administering the recombinant commensal bacterium of any one of aspects 1-81 to the subject, wherein the administration results in colonization of a native host niche by the bacterium and generation of the T cell response, wherein the T cell response is to an antigen derived from the non-native protein or peptide.
99. The method of aspect 98, wherein the colonization of the native host niche is persistent or transient.
100. The method of aspect 99, wherein the native host niche is persistently colonized, and wherein colonization is for at least 60 days, at least 112 days, at least 178 days, at least 1 year, at least 2 years, or at least 5 years.
101. The method of aspect 99, wherein the native host niche is persistently colonized, and wherein colonization is for at least 180 days.
102. The method of aspect 100 or 101, wherein the persistent colonization provides a persistent antigen source, optionally wherein the antigen stimulates an antigen-specific T cell population and produces a persistent antigen-specific T cell population.
103. The method of aspect 99, wherein the native host niche is transiently colonized, and wherein colonization is for 1 day to 60 days.
104. The method of aspect 99, wherein the native host niche is transiently colonized, and wherein colonization is for 3.5 days to 60 days.
105. The method of aspect 103 or 104, wherein the native host niche is transiently colonized, and wherein colonization is for 7 days to 28 days.
106. The method of any one of aspects 98-105, wherein colonization is determined by polymerase chain reaction or colony forming assay performed on a sample obtained from the host after 1 day, 3.5 days, 7 days, 14 days, 28 days, or 60 days after administration to the host.
107. The method of any one of aspects 98-106, wherein the administration is via a route selected from the group consisting of topical, enteral, parenteral and inhalation.
108. The method of aspect 107, wherein the route is topical.
109. The method of aspect 107, wherein the route is enteral.
110. The method of any one of aspects 98-109, wherein the T cell response comprises a CD4+T-helper response, a CD8+ cytotoxic T cell response, or a CD4+T-helper response and a CD8+ cytotoxic T cell response.
111. The method of clam 110, wherein the CD4+T-helper response is a T_{H}1 response, a T_{H}2 response, a T_{H}17 response, or a combination thereof.
112. The method of clam 110, wherein the CD4+T-helper response is a T_{H}1 response.
113. The method of clam 110, wherein the CD4+T-helper response is a T_{H}2 response.
114. The method of any one of aspects 98-109, wherein the T cell response comprises a T_{reg} response.
115. A method of treating a disease or condition in a subject, comprising: administering the recombinant commensal bacterium of any one of aspects 1-81 to the subject, wherein the administration results in colonization of a native host niche by the bacterium and generation of a T cell response, wherein the T cell response is to an antigen derived from the non-native protein or peptide, and wherein the T cell response treats the disease or condition in the subject.
116. The method of aspect 115, wherein the colonization of the native host niche is persistent or transient.
117. The method of aspect 116, wherein the native host niche is persistently colonized, and wherein colonization is for at least 60 days, at least 112 days, at least 178 days, at least 1 year, at least 2 years, or at least 5 years.
118. The method of aspect 116, wherein the native host niche is persistently colonized, and wherein colonization is for at least 180 days.
119. The method of aspect 117 or 118, wherein the persistent colonization provides a persistent antigen source, optionally wherein the antigen stimulates an antigen-specific T cell population and produces a persistent antigen-specific T cell population.
120. The method of aspect 116, wherein the native host niche is transiently colonized, and wherein colonization is for 1 day to 60 days.
121. The method of aspect 116, wherein the native host niche is transiently colonized, and wherein colonization is for 3.5 days to 60 days.
122. The method of aspect 120 or 121, wherein the native host niche is transiently colonized, and wherein colonization is for 7 days to 28 days.
123. The method of any one of aspects 115-122, wherein colonization is determined by polymerase chain reaction or colony forming assay performed on a sample obtained from the host after 1 day, 3.5 days, 7 days, 14 days, 28 days, or 60 days after administration to the host.
124. The method of aspect any one of aspects 115-123, wherein the disease or condition is an infection, a proliferative disorder, or an autoimmune disorder.
125. The method of aspect 124, wherein the infection is selected from the group consisting of a viral infection, a parasitic infection, a bacterial infection, and a fungal infection.
126. The method of aspect 124, wherein the proliferative disorder is a cancer.
127. The method of aspect 126, wherein the cancer is selected from melanoma, basal cell carcinoma, squamous cell carcinoma, testicular cancer, cervical cancer, anal cancer and nasopharyngeal cancer.
128. The method of aspect 127, wherein the cancer is melanoma.
129. The method of any one of aspects 115-128, wherein the administration is via a route selected from the group consisting of topical, enteral, parenteral and inhalation.
130. The method of aspect 129, wherein the route is topical.
131. The method of aspect 130, wherein the bacterium is S. epidermidis.
132. The method of aspect 131, wherein the disease is a cancer.
133. The method of aspect 132, wherein the cancer is melanoma.
134. The method of aspect 131 or 132, wherein the non-native protein or peptide is selected from the group consisting of a melanocyte-specific antigen and a testis cancer antigen, optionally wherein the melanocyte-specific antigen is selected from the group consisting of PMEL, TRP2 and MART-1 and optionally wherein the testis cancer antigen is selected from the group consisting of NY-ESO and MAGE-A.
135. The method of aspect 131 or 132, wherein the non-native protein or peptide comprises a neoantigen, wherein the neoantigen comprises at least one mutation that makes the non-native protein or peptide distinct from a protein or peptide encoded by a wild-type gene of the host.
136. The method of any one of aspects 83-135, wherein the bacterium is administered in combination with a high-complexity defined microbial community.
137. The method of any one of aspects 83-136, wherein the host is a mammal.
138. The method of aspect 137, wherein the mammal is a human.
139. The method of any one of aspects 83-138, wherein the method comprises (a) administering a first recombinant commensal bacterium engineered to express a first antigenic peptide comprising the non-native protein or peptide, wherein the first antigenic peptide is engineered to elicit a CD4+ T cell response, and (b) administering a second recombinant commensal bacterium engineered to express a second antigenic peptide comprising the non-native protein or peptide, wherein the second antigenic peptide is engineered to elicit a CD8+ cytotoxic T cell response.
140. The method of aspect 139, wherein the first antigenic peptide comprises a signal sequence peptide that directs secretion of the first antigenic peptide from the bacterium following expression.
141. The method of aspect 140, wherein the second antigenic peptide comprises a signal sequence peptide that directs covalent attachment of the second antigenic peptide to a cell wall of the bacterium following expression.
142. The method of any one of aspects 83-141, wherein the method further comprises co-administering one or more additional agents.
143. The method of aspect 142, wherein the one or more additional agents comprises one or more checkpoint inhibitors.
144. The method of any one of aspects 83-143, wherein a distal adaptive immune response is produced.
145. The method of aspect 144, wherein the distal adaptive immune response is distal from the site of administration.
146. The method of aspect 144 or 145, wherein the distal adaptive immune response is distal from the native host niche.
147. The method of aspect 145 or 146, wherein the distal adaptive immune response comprises an immune response in an organ that is not the organ of the site of administration and/or the native host niche.
148. The method of any one of aspects 145-147, wherein the site of administration and/or the native host niche comprises skin.
149. The method of any one of aspects 144-148, wherein the distal adaptive immune response comprises an antitumor response.
150. The method of aspect 149, wherein the antitumor response targets a metastasis.
151. A bacterial surface display system comprising: (a) a fusion protein comprising a cell-surface tethering moiety; (b) a bacterium; and (c) a protein or gene encoding the same capable of catalyzing a covalent attachment of the cell-surface tethering moiety to a cell wall protein or outer membrane protein of the bacterium thereby displaying the fusion protein on a bacterial surface.
152. A bacterial surface display system comprising: (a) a fusion protein comprising a cell-surface tethering moiety and (b) a bacterium, wherein the fusion protein is covalently attached to a cell wall protein or outer membrane protein via the cell-surface tethering moiety, and wherein the covalent attachment was catalyzed by a protein capable of catalyzing attachment of the cell-surface tethering moiety to the cell wall protein or outer membrane protein of the bacterium.
153. The bacterial surface display system of aspect 151 or 152, wherein the cell-surface tethering moiety comprises a Sortase A (SrtA) motif and the protein capable of catalyzing the covalent attachment is a SrtA protein.
154. The bacterial display system of aspect 153, wherein the SrtA motif is derived from S. aureus.
155. The bacterial surface display system of aspect 153 or 154, wherein the SrtA motif comprises the amino acid sequence LPXTG.
156. The bacterial surface display system of any one of aspects 153-155, wherein the SrtA protein is derived from S. aureus.
157. The bacterial surface display system of any one of aspects 151-156, wherein the fusion protein comprises an antigenic protein.
158. The bacterial surface display system of aspect 157, wherein the antigenic protein comprises a protein or peptide associated with a host disease or condition.
159. The bacterial surface display system of aspect 157 or 158, wherein the protein or peptide is associated with an infection, a proliferative disorder, or an autoimmune disorder.
160. The bacterial surface display system of any one of aspects 157-159, wherein upon administration of the bacterium to the host resulting in colonization of a native host niche by the bacterium, the host mounts an adaptive immune response to the non-native protein or peptide, wherein the adaptive immune response is a T cell response.
161. The bacterial surface display system of any one of aspects 151-160, wherein the bacterium is a commensal.
162. The bacterial surface display system of any one of aspects 151-161, wherein the bacterium is a Gram-positive bacterium.
163. The bacterial surface display system of aspect 162, wherein the Gram-positive bacterium is selected from the group consisting of Staphylococcus epidermidis, Faecalibacterium sp., Corynebacterium spp., Eubacterium limosum, Ruminococcaceae bacterium cv2, Clostridium sp., Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus AGR2154, Butyrate-producing bacterium SSC/2, Clostridium sp. ASF356, Coprobacillus sp. D6 cont1.1, Eubacterium sp. 3_1_31 cont1.1, Erysipelotrichaceae bacterium 213, Ruminococcus bromii L2-63, Firmicutes bacterium ASF500, Firmicutes bacterium ASF500, Bifidobacterium animalis subsp. lactis ATCC 27673, and Bifidobacterium breve UCC2003.
164. The bacterial surface display system of aspect 163, wherein the Gram-positive bacterium is selected from the group consisting of Staphylococcus epidermidis, Faecalibacterium sp., Corynebacterium spp., and Clostridium sp.
165. The bacterial surface display system of aspect 164, wherein the bacterium is selected from the group consisting of Staphylococcus epidermidis and Corynebacterium spp.
166. The bacterial surface display system of aspect 165, wherein the bacterium is S. epidermidis NIHLM087.
167. The bacterial antigen display system of any one of aspects 151-161, wherein the commensal bacterium is selected from the group consisting of: Corynebacterium tuberculostearicum, Corynebacterium accolens, Corynebacterium amycolatum, Corynebacterium aurimucosum, Corynebacterium propinquum, Corynebacterium pseudodiphtheriticum, Corynebacterium granulosum, Cutibacterium acnes, Cutibacterium avidum, Dolosigranulum pigrum, Finegoldia magna, Moraxella catarrhalis, Moraxella nonliquefaciens, Haemophilus influenzae, Haemophilus aegyptius, Rothia mucilaginosa, Streptococcus pyogenes, Streptococcus agalactiae, Streptococcus gordonii, Neisseria lactamica, Neisseria cinerea, Neisseria mucosa, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium breve ATCC 15700, Bifidobacterium longum, Veillonella parvula, Gardnerella vaginalis, Atopobium vaginae, Prevotella bivia, Mobiluncus mulieris, Mageeibacillus indolicus, Prevotella buccalis, Enterococcus faecium, Lactococcus lactis, Ruminococcus gnavus JCM6515, and Eubacterium limosum.
168. The bacterial surface display system of aspect 167, wherein the commensal bacterium is selected from the group consisting of: a bacterium having ATCC accession number 35692, 49725, 49726, 49368, 700975, 700540, 51488, 10700, 25564, 51277, 11827, 25577, 49753, 51524, 29328, 25238, 25240, 19976, 51907, 11116, 25296, 19615, 12344, BAA-611, 13813, 10558, 23970, 14685, 19696, 33820, 25258, 19992, 55195, 4356, 33200, 7469, 393, 7995D-5, 23272, 11741, 15700, 15697, 10790, 17745, 14018, BAA-55, 29303, 35243, BAA-2120, 35310, 19434, 19435, 29149, and 8486.
169. The bacterial surface display system of aspect 167, wherein the commensal bacterium is selected from the group consisting of: Lactobacillus casei, Lactococcus lactis, Streptococcus gordonii, Lactobacillus crispatus, Lactobacillus iners, Cutibacterium acnes, Streptococcus agalactiae, Ruminococcus gnavus, Neisseria lactamica, Bifidobacterium breve, and Bifidobacterium longum.
170. The bacterial surface display system of aspect 169, wherein the commensal bacterium is selected from the group consisting of: a bacterium having ATCC accession number 393, 19435, 35105, 33820, 55195, 6919, 13813, 23970, 15700, and 15707, and a bacterium having an accession number JCM6515.
171. The bacterial surface display system of any one of aspects 151-170, wherein the fusion protein further comprises an antigen-presenting cell (APC) targeting moiety, optionally wherein the APC targeting moiety comprises a CD11b or a MHC II targeting moiety.
172. The bacterial surface display system of aspect 171, wherein the APC targeting moiety comprises a nanobody (VHH) antibody binding domain, optionally wherein the VHH antibody binding domain comprises the sequence of SEQ ID NO:33 or SEQ ID NO:34.
173. The bacterial surface display system of any one of aspects 151-172, wherein the host is a mammal.
174. The bacterial surface display system of aspect 173, wherein the host is a human.
175. A pharmaceutical composition comprising the bacterial surface display system of any one of aspects 151-174 and an excipient.
176. The pharmaceutical composition of aspect 175, wherein the pharmaceutical composition comprises a high-complexity defined microbial community.
177. A method for generating an antigen-presenting cell displaying an antigen derived from a non-native protein or peptide, comprising: administering the bacterial surface display system of any one of aspects 151-174 or the pharmaceutical composition of aspect 175 or 176 to a subject, wherein the administration results in colonization of the native host niche by the bacterium, internalization of the bacterium or the non-native protein or peptide by an antigen-presenting cell, and presentation of the antigen by the antigen-presenting cell.
178. A method for generating a T cell response in a subject, comprising: administering the bacterial surface display system of any one of aspects 151-174 or the pharmaceutical composition of aspect 175 or 176 to a subject, wherein the administration results in colonization of a native host niche by the bacterium and generation of the T cell response, wherein the T cell response is to an antigen derived from the non-native protein or peptide.
179. A method of treating a disease or condition in a subject, comprising: administering the bacterial surface display system of any one of aspects 151-174 or the pharmaceutical composition of aspect 175 or 176 to a subject, wherein the administration results in colonization of a native host niche by the bacterium and generation of a T cell response, wherein the T cell response is to an antigen derived from the non-native protein or peptide, and wherein the T cell response treats the disease or condition in the subject.
180. The method of any one of aspects 177-179, wherein the colonization of the native host niche is persistent or transient.
181. The method of aspect 180, wherein the native host niche is transiently colonized, and wherein colonization is for 1 day to 60 days.
182. The method of aspect 181, wherein the native host niche is transiently colonized, and wherein colonization is for 3.5 days to 60 days.
183. The method of aspect 181 or 182, wherein the native host niche is transiently colonized, and wherein colonization is for 7 days to 28 days.
184. The method of any one of aspects 177-183, wherein colonization is determined by polymerase chain reaction or colony forming assay performed on a sample obtained from the host after 1 day, 3.5 days, 7 days, 14 days, 28 days, or 60 days after administration to the host.
185. The method of any one of aspects 177-184, wherein the administration results in interaction of the bacterium with a native immune system partner cell.
186. The method of aspect 185, wherein the native immune system partner cell is the antigen-presenting cell.
187. The method of aspect 186, wherein the antigen-presenting cell is selected from the group consisting of a dendritic cell, a macrophage, a B-Cell, and an intestinal epithelial cell.
188. The method of any one of aspects 177-187, wherein the native host niche is selected from the group consisting of the gastrointestinal tract, respiratory tract, urogenital tract, and skin.
189. The method of any one of aspects 177-188, wherein the presentation is within an MHC-II complex.
190. The method of any one of aspects 177-188, wherein the presentation is within an MHC-I complex.
191. The method of any one of aspects 177-190, wherein the bacterial surface display system is administered in combination with a high-complexity defined microbial community.
192. The method of any one of aspects 177-191, wherein the administration is via a route selected from the group consisting of topical, enteral, parenteral and inhalation.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1—Expression of OVA in Bacteroides Strains

Antigenic epitope coding sequences were cloned into the pWW3837 vector (Genbank #KY776532), (see Whitaker et al., “Tunable Expression Tools Enable Single-Cell Strain Distinction in the Gut Microbiome,” Cell 169, 538-546, Apr. 20, 2017) by Gibson assembly. The vector was electroporated into E. coli S17 lambda pir donor strains. E. coli donor strains were co-cultured overnight with recipient bacteria for conjugation on a BHI blood plate. Biomass was scraped and plated onto BHI Blood+erm/gent plates. Positive colonies were screened by colony-PCR.

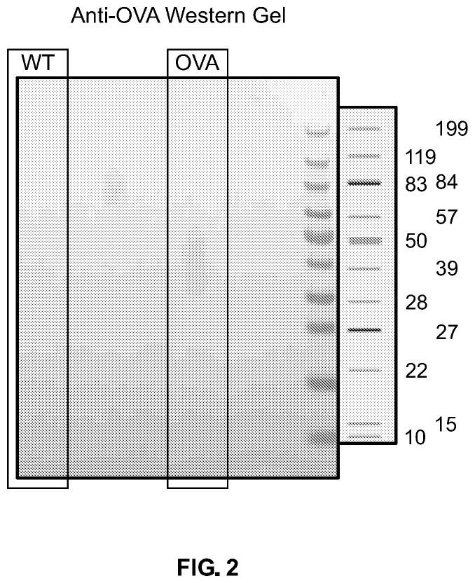

As shown in FIG. 2, Western blotting data demonstrates that Bacteroides thetaiotaomicron engineered to express an OVA epitope (“OVA”) showed detectable levels of OVA whereas wild-type B. thetaiotaomicron (“WT”; negative control) shows no signal.

### Example 2—In Vitro Induction of OVA-Specific T Cells by Recombinant Bacteroides Strains

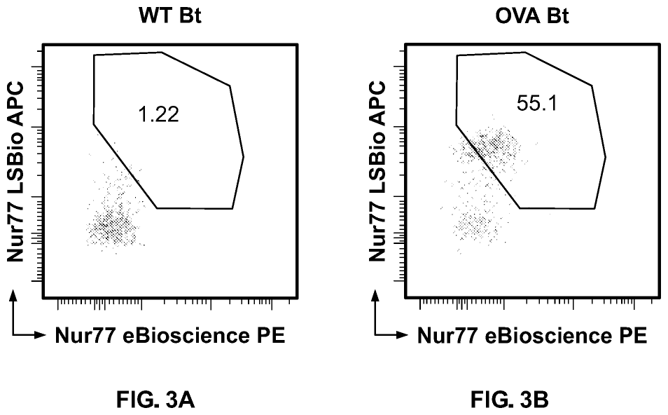

OVA-specific T cells isolated from the spleens of OTII transgenic mice were co-cultured for 4 hours with B16-FLT3L stimulated DCs and OVA⁺ B. thetaiotaomicron (FIG. 3B) or WT B. thetaiotaomicron (FIG. 3A). As shown in FIG. 3B, OTII T cells cultured with OVA*B. thetaiotaomicron upregulate the expression of Nur77 (two different Nur77 antibodies were used to increase specificity).

### Example 3—Expression of MOG Fusion Peptides in Recombinant Bacteroides Strains

Myelin oligodendrocyte glycoprotein (MOG) 35-55 peptide sequences were cloned into the pWW3837 vector, electroporated into E. coli donor strains, and conjugated with commensal recipient strains using an analogous method as described in EXAMPLE 1.

Commensal bacterial strains and expression constructs are summarized in Table 5.

TABLE 5
MOG-Expressing Bacterial Strains and Constructs

| | | | Location of MOG |
|---|---|---|---|
| | | | Peptide Relative to |
| Strain | | Native Fusion | Native Fusion |
| Name | Commensal Strain | Protein | Protein |
| BT_W | Bacteroides | — | — |
| | thetaiotaomicron | | |
| | VPI-5482 | | |
| BT_ | Bacteroides | BT0455 | N-Terminal |
| MOG#1 | thetaiotaomicron | (Sialidase) | |
| | VPI-5482 | | |
| BT_ | Bacteroides | BT1279 (Anti- | N-Terminal |
| MOG#5 | thetaiotaomicron | Sigma Factor) | |
| | VPI-5482 | | |
| BV_W | Bacteroides vulgatus | — | — |
| | ATCC 8482 | | |
| BV_ | Bacteroides vulgatus | BT0455 | N-Terminal |
| MOG#1 | ATCC 8482 | (Sialidase) | |
| BV_ | Bacteroides vulgatus | BT1279 (Anti- | N-Terminal |
| MOG#5 | ATCC 8482 | Sigma Factor) | |
| BF_W | Bacteroides finegoldii | — | — |
| | DSM 17565 | | |
| BF_ | Bacteroides finegoldii | BT0455 | N-Terminal |
| MOG#1 | DSM 17565 | (Sialidase) | |
| BF_ | Bacteroides finegoldii | BT1279 (Anti- | N-Terminal |
| MOG#5 | DSM 17565 | Sigma Factor) | |

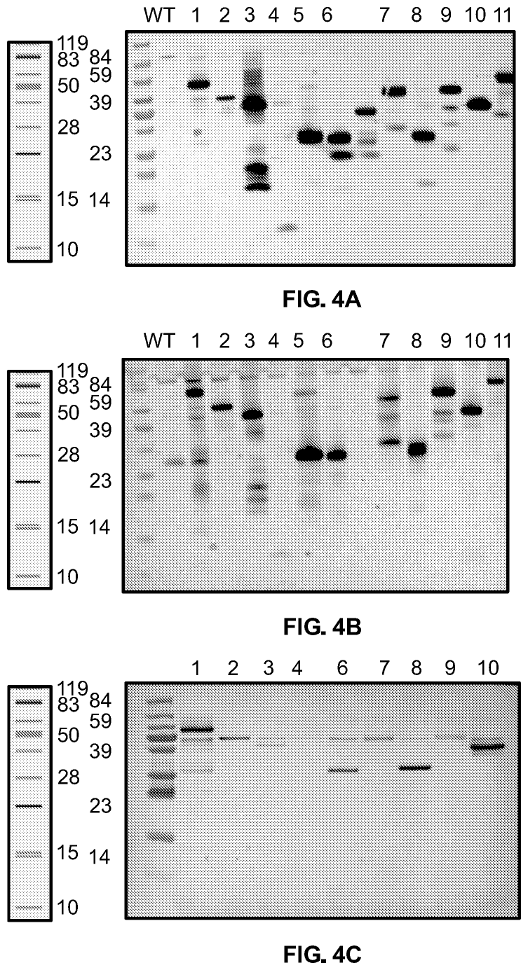

As shown in FIG. 4, Western blotting data using an anti-FLAG antibody demonstrates that B. thetaiotaomicron (FIG. 4A) engineered to express FLAG-tagged MOG35-55 peptide (BT_MOG #1 and BT_MOG #5, lanes 1 and 5, respectively), Bacteroides vulgatus (FIG. 4B) engineered to express FLAG-tagged MOG 35-55 peptide (BV_MOG #1 and BT_MOG #5, lanes 1 and 5, respectively), and Bacteroides finegoldii (FIG. 4C) engineered to express FLAG-tagged MOG 35-55 peptide (B3F_MOG #1, lane 1), all showed detectable levels of MOG peptide, whereas wild-type B. thetaiotaomicron, B. vulgatus, and B. finegoldii (FIG. 4A; WT, FIG. 4B; WT, and not shown, respectively), did not show any signal.

### Example 4—In Vitro Induction of MOG-Specific T Cells by Recombinant Bacteroides Strains

To expand splenic dendritic cells (DCs), CD45.1 C571BL/6 (The Jackson Laboratory, strain #002014) mice were injected subcutaneously at the flank with 5×10⁶ B16 melanoma cells III overexpressing Flt3L. On day 11, spleens were harvested, digested using a spleen dissociation kit (Miltenyi) and splenic DCs were purified using CD11c microbeads (Miltenyi).

To prepare bacterial antigen, live, recombinant B. thetaiotaomicron expressing MOG35-55 peptide (prepared by a method analogous to the method described in Example 3) were washed and resuspended in complete T cell media (DMEM, 10% FBS, 10 mM HEPES, 50 μM 2-ME). Heat-killing was performed at 65° C. for 15 minutes and loss of bacterial viability was confirmed by culturing. Autoclaved antigen was prepared by autoclaving bacterial suspension at 121° C. for 45 minutes at 15 psi. MOG-specific T cells were isolated and purified from spleens and peripheral lymph nodes of 2D2 TCR-Tg mice (The Jackson Laboratory, strain #006912) using a CD4+ T cell isolation kit (Miltenyi).

To prepare APC-T cell co-cultures, 2×10⁵ splenic DCs were pulsed with live, heat-killed or autoclaved bacteria at a multiplicity of infection (MOI) of 10-50 or 40 μg/ml of total protein for 4 hours at 37° C. 2×10⁵ MOG-specific 2D2 CD4 T cells were added to APCs. On day 2 post-co-culture, cells were harvested, stained with fluorochrome conjugated antibodies for CD45.1, CD45.2, TCRb, CD4, CD25, CD44, CD69 (ThermoFisher Scientific or BioLegend), and/or cell trace violet (CTV) and assessed by flow cytometry (Attune NxT). Live cells were excluded by Live/Dead Aqua (ThermoFisher Scientific). Data analysis was performed using FlowJo v10.

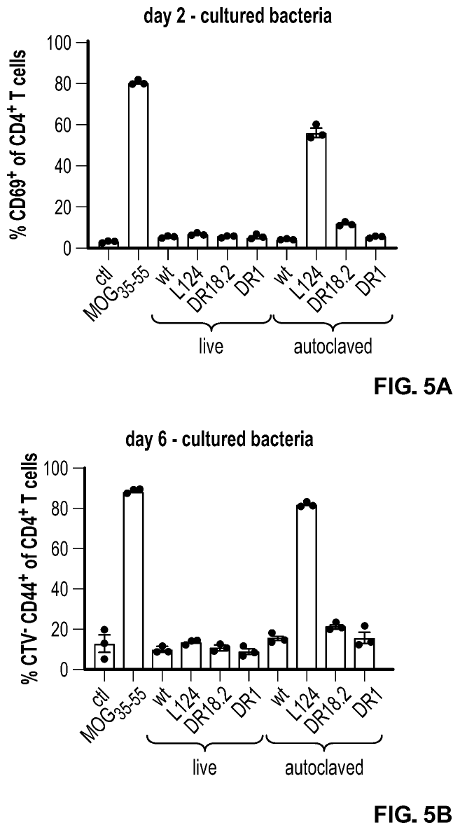

As shown in FIGS. 5A and 5B, recombinant B. thetaiotaomicron strains expressing MOG35-55 peptide (L124, DR18.2, and DR1) induced a greater antigen-specific induction of CD4+ T cells than wild-type B. thetaiotaomicron (wt).

### Example 5—In Vivo Induction of MOG-Specific T Cells by Recombinant Bacteroides Strains

The Experimental Autoimmune Encephalomyelitis (EAE) model was used as a murine model for multiple sclerosis (MS). Germ-free 8-10 week old C57BL/6 mice or C57BL/6-Tg (Tcra2D2,Tcrb2D2)1Kuch/J mice were orally inoculated with MOG35-55 peptide-expressing bacteria (BVF-MOG=a mixture of B. vulgatus and B. finegoldii expressing MOG35-55) or wild-type commensal bacteria as a negative control (BVF-WT=a mixture of wild-type B. vulgatus and B. finegoldii) on day one. Wild-type and recombinant bacterial strains were obtained as previously described in Example 3. On day 14, these mice were subcutaneously immunized with the Hooke Kit™ MOG35-55/CFA emulsion (EK-2110, Hooke Labs, St Lawrence, MA, USA), which contains 200 g MOG35-55 emulsified in 200 μL Complete Freund's Adjuvant (CFA). On day 14, 2 hours after MOG35-55/CFA immunization, 200 ng of pertussis toxin (PTX) in phosphate buffered saline (PBS) was injected into the intraperitoneal cavity of each mouse. On day 15, 200 ng of pertussis toxin (PTX) in PBS was injected intraperitoneally. EAE scores and body weights were assessed daily from day 15 to day 34 in order to evaluate the severity and stage of the disease. To alleviate the distress from this experiment, mice were euthanized when reaching a score of 3.5. Score 0 means no obvious changes in motor functions. Score 0.5 is a distal paralysis of the tail; score 1 complete tail paralysis; score 1.5 mild paresis of one or both hind legs; score 2 severe paresis of hind legs; score 2.5 complete paralysis of one hindleg; score 3 complete paralysis of both hind legs and score 3.5 complete paralysis of hind legs and paresis of one front leg. Mice reaching scores ≥3.5 were euthanized.

On day 35, mice were euthanized; spinal cord samples were prepared for histological analysis; inguinal lymph nodes were collected, washed with PBS, dissociated to obtain a cell suspension, fixed used a FoxP3 staining buffer set (eBioscience), and stained with various fluorescently-labelled antibodies for flow cytometry analysis on a BD-LSRII instrument.

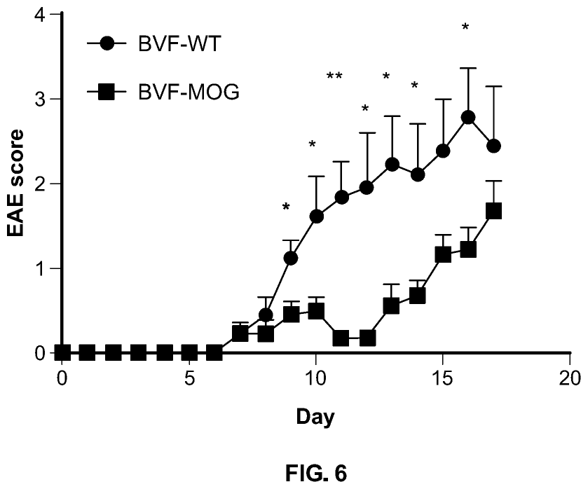

As shown in FIG. 6, mice administered with a mixture of recombinant B. vulgatus and B. finegoldii expressing MOG35-55 peptide (BVF-MOG) had a significantly reduced EAE score as compared to mice administered with a mixture of wild-type B. vulgatus and B. finegoldii (BVF-WT). * p≤0.05, ** p≤0.01. Results are from three independent experiments.

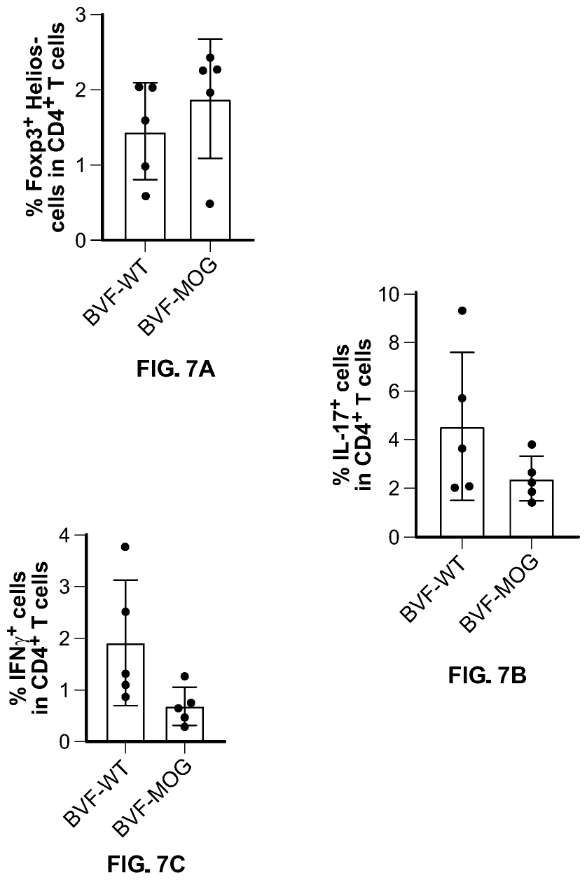

As shown in FIG. 7A, mice were administered with a mixture of recombinant B. vulgatus and B. finegoldii expressing MOG35-55 peptide (BVF-MOG) had an increased number of lymph node FoxP3+ Helios-CD4+ T cells as compared to mice administered with a mixture of wild-type B. vulgatus and B. finegoldii (BVF-WT). Mice administered with a mixture of recombinant B. vulgatus and B. finegoldii expressing MOG35-55 peptide (BVF-MOG) also exhibited fewer IL17+CD4+ T cells (FIG. 7B) and IFN-7+CD4+ T cells (FIG. 7C) as compared to mice administered with a mixture of wild-type B. vulgatus and B. finegoldii (BVF-WT).

### Example 6—In Vitro Induction of OVA Specific T Cells by Recombinant Staphylococcus Strains

A Staphylococcus/E. coli shuttle vector with a constitutive promoter (pLI50-Ppen, published in Swoboda et al., ACS Chem Biol. 2009) was fused to the ribosome binding site from the S. aureus delta-hemolysin (hld) gene, which promotes strong, constitutive translation in S. aureus and S. epidermidis (Malone et al., J Microbiol Methods 2009.). In some cases, pLI50-Ppen was modified to be a minicircle plasmid, denoted pLI50mini, using a published strategy (Johnston et al., PNAS 2019).

Four forms of the OVA antigen were designed using the in silico prediction methods described in Chun et al. J Exp Med 2001: (i) the full-length protein, (ii) a 1× repeat of an MHC-I-binding antigen from OVA (with amino acid sequence SIINFEKL; “1×”), (iii) a 3× repeat of SIINFEKL (“3×”), or (iv) a concatemer of three predicted H2-M3-binding peptides from OVA (“3pep”).

Next, S. epidermidis strains for cell-wall displayed antigen were produced. In these strains, OVA, 1×, 3×, or 3pep were spliced between two domains of S. aureus protein A: an N-terminal signal peptide and a C-terminal cell wall-spanning region, yielding wOVA, wOVA1x, wOVA3x, and wOVA3pep.

Pepboy mice were injected intraperitoneally with B16-melanoma producing Flt3L to stimulate overall dendritic cell production. After about 10-13 days, splenic dendritic cells were isolated with CD11c magnetic beads. These dendritic cells were incubated with heat-killed bacteria for 2.5 hours at 37° C. T cells isolated from spleens of transgenic mice (OT-I or OT-II) were isolated with a pan-T cell isolation kit (Miltenyi). After dendritic cell/bacteria incubation, T cells of interest were added to the dendritic cell co-cultures at a 10:1 or higher dendritic cell to T cell ratio and co-cultured at 37° C. for another 3.5 hours. After co-culture, cells were collected for fixation and staining for cell surface markers, intracellular transcription factors, and intracellular Nur77 expression for flow cytometry analysis. Nur77 expression was used as a marker for antigen-specific TCR binding and activation of the T cell during co-culture.

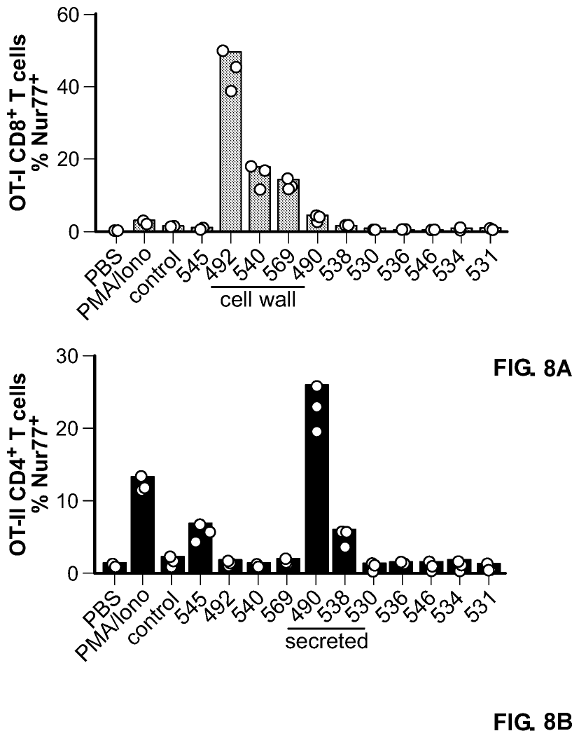

As shown in FIG. 8A, recombinant S. epidermidis strains expressing OVA fusion proteins at the cell wall (strains 492, 540, and 569) increased the proportion of Nur77-expressing CD8+ T cells in co-culture. In contrast, as shown in FIG. 8B, strains 492, 540, and 569 did not increase the proportion of Nur77-expressing CD4+ T cells.

### Example 7—In Vitro Induction of PMEL Specific T Cells by Recombinant Staphylococcus Strains

S. epidermidis strains displaying the melanocyte-specific antigen, PMEL, at the bacterial cell wall were produced by an analogous method as previously described in EXAMPLE 6.

In vitro mixed bacteria/APC/T Cell Reactions were performed as previously described in EXAMPLE 6, but using PMEL-expressing S. epidermidis bacterial strains instead of OVA-expressing S. epidermidis strains, and using 8rest CD8 T Cells instead of OT-I or OT-II T Cells.

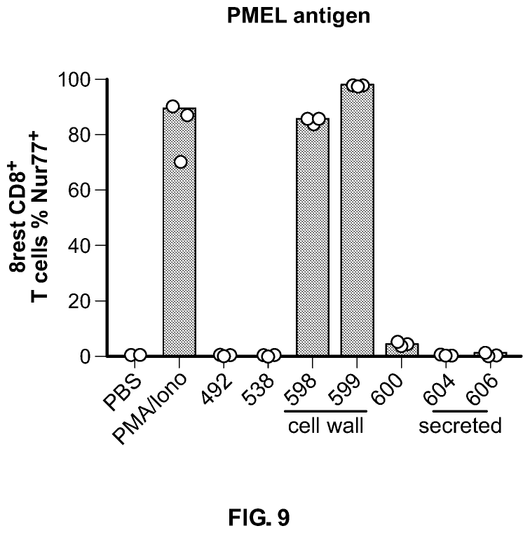

Similarly to OVA-expressing recombinant S. epidermidis strains, as shown in FIG. 9, PMEL-expressing recombinant S. epidermidis strains increased the proportion of Nur77-expressing CD8+ T cells in co-culture.

### Example 8—In Vivo Induced Tumor Killing of OVA+ Melanoma by OVA-Expressing S. epidermidis

C57BL/6 female mice between the ages of 8-12 weeks were used for all in vivo melanoma experiments. 1-3×10⁵ melanoma cells were injected subcutaneously or intraperitoneally for a local or metastatic model of melanoma progression. The melanoma cells were either a B16F10 cell line expressing OVA, a B16F10 cell line expressing OVA and luciferase, or ATCC B16F0-luciferase and B16F10-luciferase cell lines. Injection of melanoma occurred up to 1 week before or 2 weeks after topical administration of mice with tumor antigen-expressing S. epidermidis. For mice injected with luciferase-expressing B16 melanoma, in vivo imaging was performed by injecting mice with 150 mg/kg of D-luciferin in sterile PBS followed by imaging under isoflurane anesthesia using an IVIS Lumina or Lago imager.

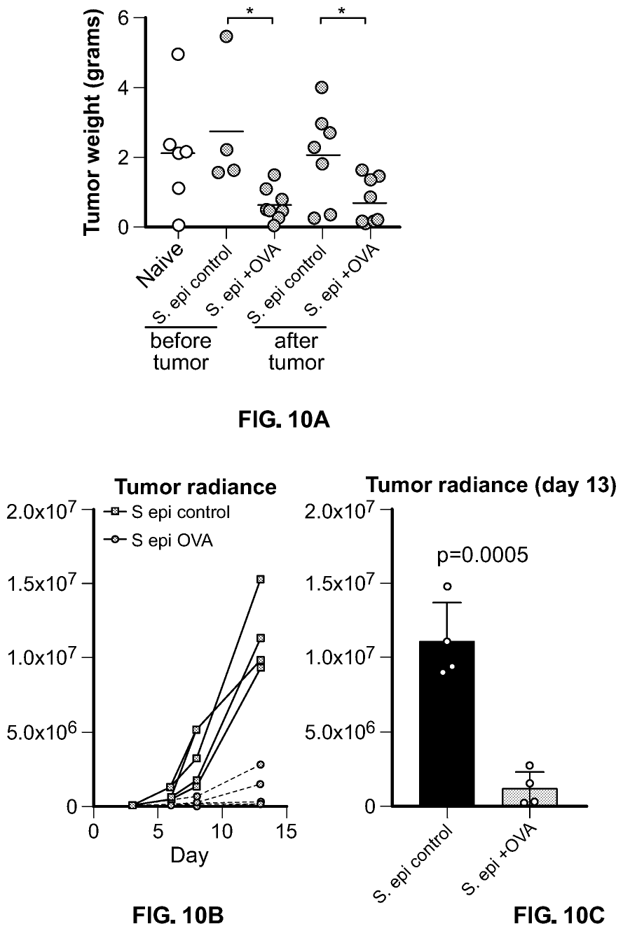

As shown in FIG. 10, topical administration of OVA-expressing S. epidermidis both prior to tumor injection and after tumor injection, resulted in a significant reduction in tumor weight (FIG. 10A) in mice as compared to mice treated with wild-type control S. epidermidis. * p<0.05. Similarly, topical administration of OVA-expressing S. epidermidis 1 to 3 days after tumor injection of luciferase-expressing melanoma cells, resulted in a significant reduction in tumor radiance/luminescence as compared to mice treated with wild-type control S. epidermidis (FIG. 10B, and FIG. 10C).

### Example 9—In Vitro Induction of Type-Specific T Cell Activation Using Recombinant S. epidermidis

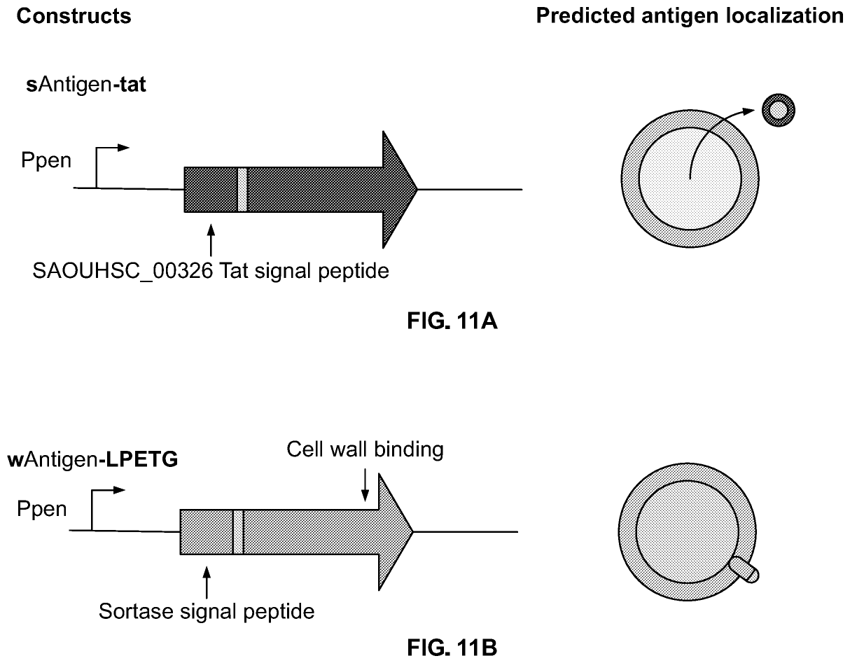

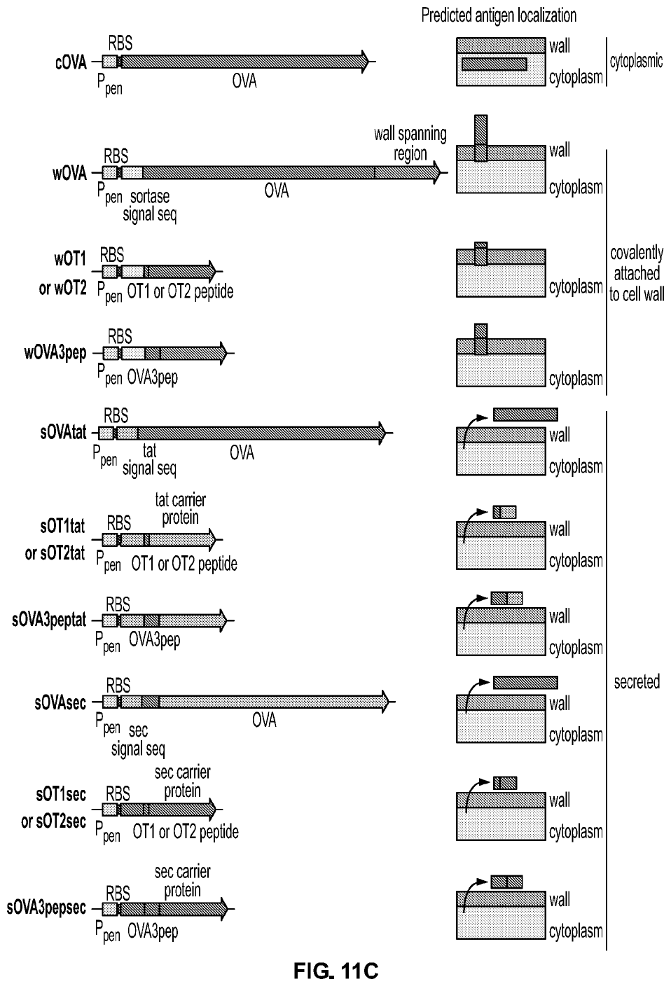

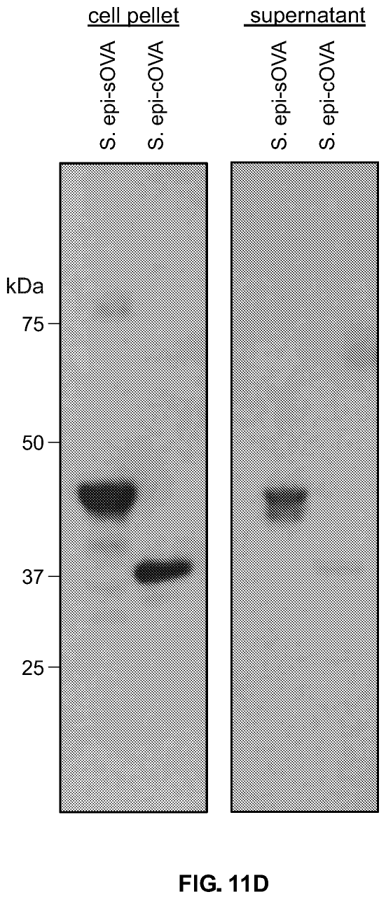

An in vitro cell culture model was used to test the induction of antigen-specific immunity in mice inoculated with recombinant bacteria expressing fusion proteins containing tumor antigens. FIG. 11A and FIG. 11B are schematic diagrams illustrating the construct designs used to express fusion proteins containing tumor antigens having specific bacterial sub-cellular localizations. The tat expression system, in which the antigen fragment is inserted into a tat carrier, results in secretion of the antigen fused with a tat carrier peptide (FIG. 11A). The cell wall-anchoring expression system, in which the antigen fragment is fused to a sortase signal peptide and cell wall-spanning peptide, results in the antigen being targeted to the bacterial cell wall (FIG. 11B). FIG. 11C shows schematic diagrams illustrating the design of specific constructs to express the OVA antigen. The most basic construct (cOVA) results in OVA localization within the cytoplasm. Addition of an N-terminal sortase signal sequence and a C-terminal wall-spanning region to OVA or fragments OT1, OT2, or OT3pep, result in the anchoring of the expressed antigen to the outer surface of the bacterial cell. By contrast, addition of an N-terminal sec or tat signal sequence results in secretion of the expressed antigen. For peptide fragments, a C-terminal carrier protein is added to promote antigen secretion. Production of the full-length OVA constructs was assessed by Western blot (FIG. 11D). Notably, although S. epidermidis has a well-described Sec secretion system and no discernible Tat secretion system, the Tat signal peptide facilitated efficient production and secretion of OVA.

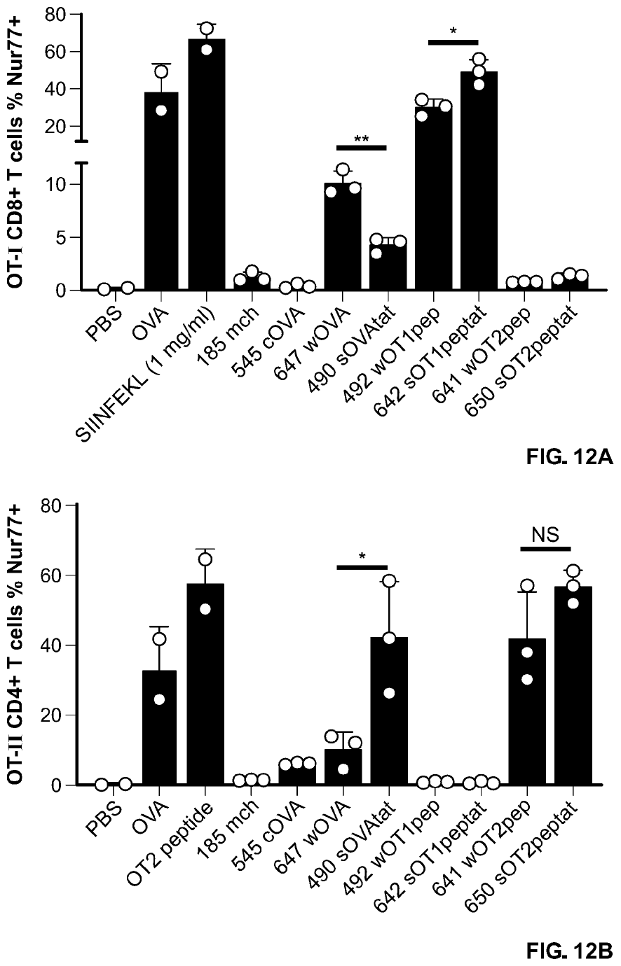

Mouse splenic dendritic cells were inoculated with recombinant S. epidermidis expressing control or antigen-containing constructs. FIG. 12 shows the activation of cultured CD8+ T cells (FIG. 12A) and CD4+ T cells (FIG. 12B) as measured by expression of Nur77, a marker of T cell activation. OT-I, a known activator of CD8+ T cells caused robust induction of Nur77 in CD8+ T cells, and OT-II, an activator of CD4+ T cells, similarly induced a robust activation of CD4+ T cells. CD8+ T cells were not strongly activated by OT-II, nor were CD4+ T cells strongly activated by OT-I, indicating a specific response of T cell types to particular antigens.

### Example 10—In Vivo Activation of Anti-Tumor Immunity in Mice with Recombinant S. epidermidis

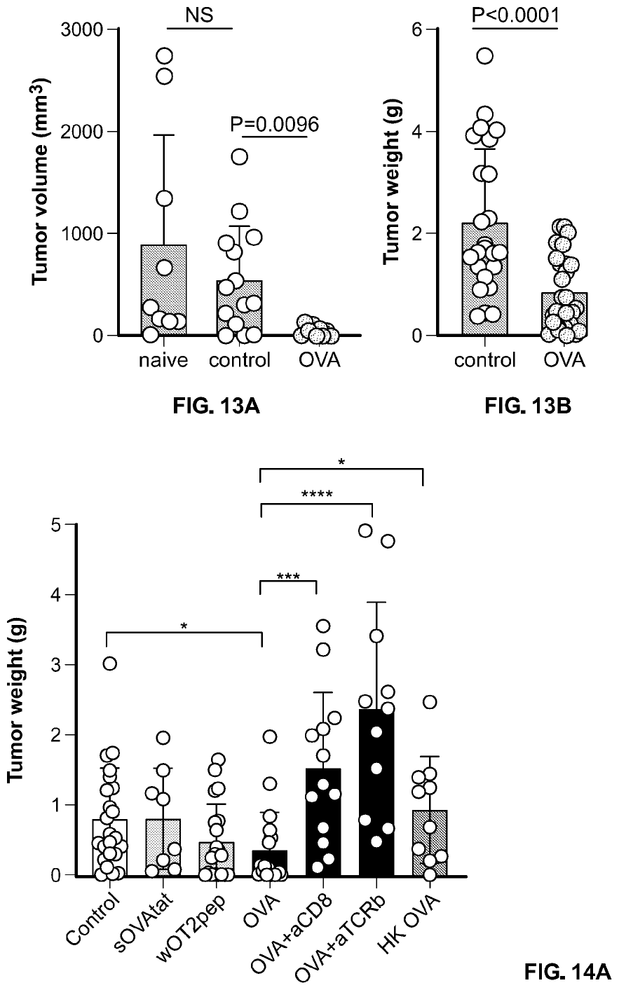

A subcutaneous xenograft model was used to test the ability of recombinant S. epidermidis to induce anti-tumor immunity against OVA-positive tumor cells. Mice were inoculated with S. epidermidis engineered with the OVA antigen construct or control for one week prior to subcutaneous xenograft with OVA-positive B16F10 melanoma cells. FIG. 13 shows analysis of tumor volume (FIG. 13A) and weight (FIG. 13B) 21-23 days post-xenograft, demonstrating significantly reduced tumor volumes and weight in mice inoculated with OVA-expressing bacteria compared to control.

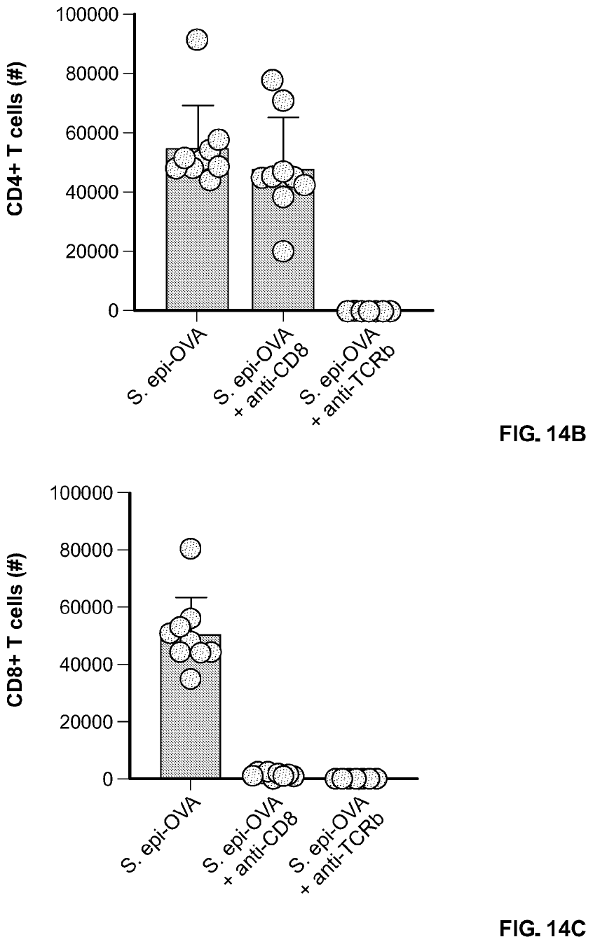

Expression of specific constructs in S. epidermidis was used to assess the relative contributions of T cell types in antigen-specific anti-tumor immunity. Mice were inoculated with bacteria expressing secreted OVA (sOVAtat), wall-attached OT1 (wOVApep), both live sOVAtat and wOVApep (OVA), or both heat-killed sOVAtat and wOVApep (HK OVA), for one week prior to subcutaneous xenograft with OVA-positive B16F0 melanoma cells. Groups of mice inoculated with both live sOVAtat and wOVApep bacterial strains (OVA) were additionally treated with antibodies targeting either CD8+ T cells (OVA+aCD8) or T cell receptor (TCR) (OVA+aTCRb). FIG. 14A shows that significant reduction in tumor weight was only seen in mice treated with both live sOVAtat and wOVApep bacterial strains. This reduction in tumor weight was prevented by co-treatment with CD8+ T cell or TCR-targeting antibodies, indicating that induction of both CD8+ and CD4+ T cells is necessary for anti-tumor immunity. Treatment with heat-killed bacteria did not result in significant reduction of tumor weight, indicating that the engineered bacteria were not simply a source of antigen and adjuvant but that bacterial viability and potentially persistent antigen exposure was generally needed for the immune stimulatory response. Antibody-mediated depletion of CD8+ T cells or all TCRβ+ cells (FIG. 14B and FIG. 14C) eliminated the antitumor effect, consistent with a role for CD8+ and CD4+ T cells in the S. epi-OVA-induced response (FIG. 14A, FIG. 14B, and FIG. 14C).

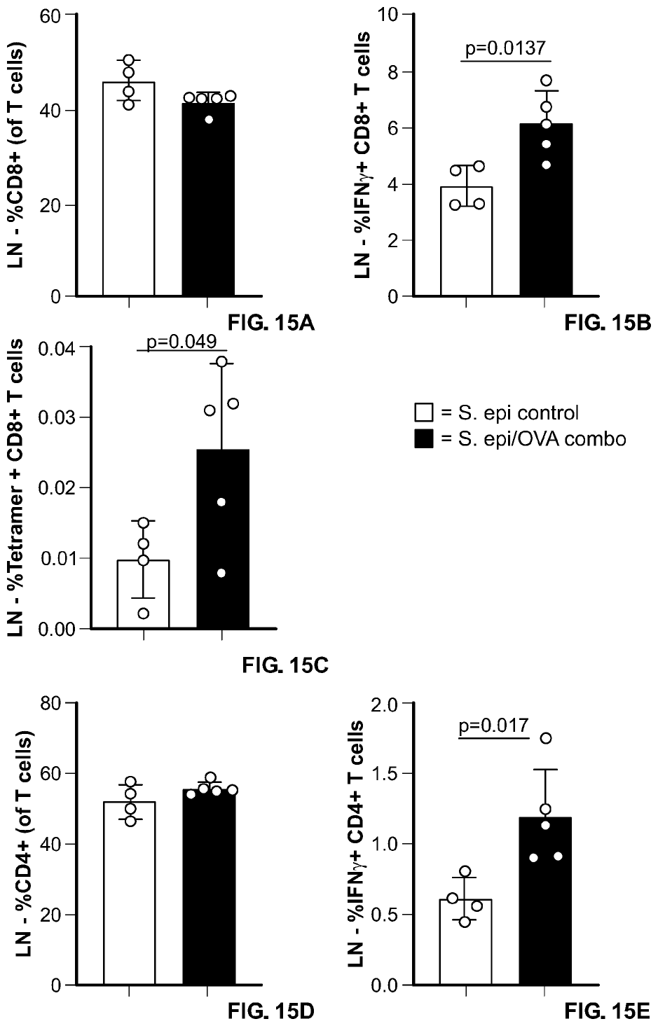

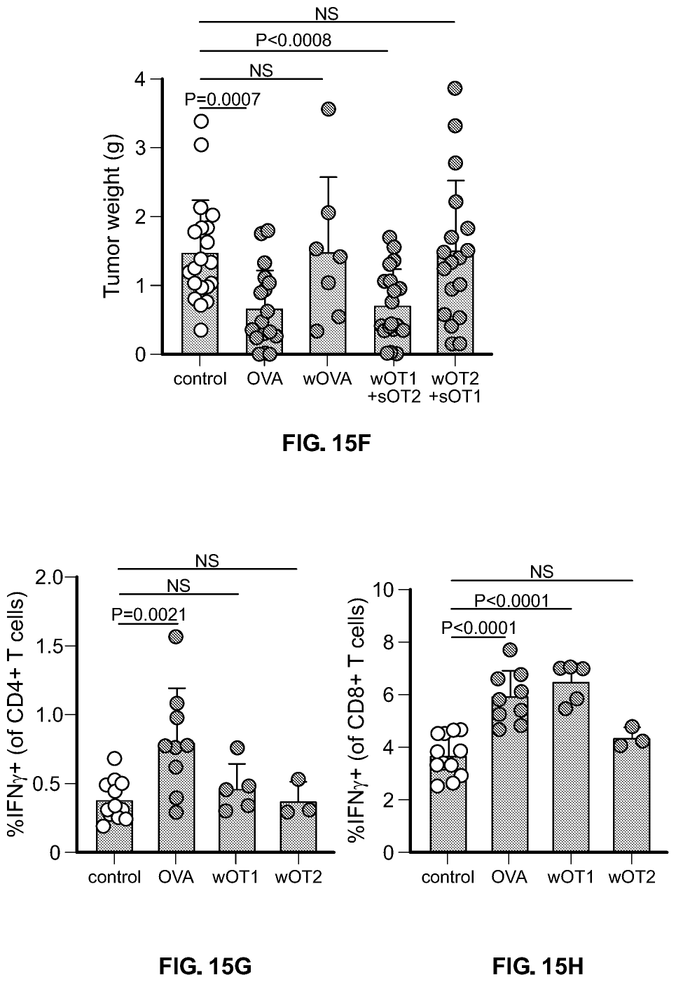

Analysis of T cells within tumor-draining lymph nodes provides an indication of antigen-specific activation of both CD8+ and CD4+ T cells in mice topically inoculated with recombinant S. epidermidis. Mice were inoculated with S. epidermidis engineered to express the OVA antigen constructs or control for one week prior to subcutaneous xenograft with OVA-positive B16-F0 melanoma cells. As shown in FIG. 15B and FIG. 15E, the percentage of activated IFNγ-expressing CD8+ T cells and CD4+ T cells, respectively, increased in tumor-draining lymph nodes following colonization with S. epi-OVA but not S. epi-control. As shown in FIG. 15C, the percentage of H2-Kb/SIINFEKL tetramer staining CD8+ T cells also increased in tumor-draining lymph nodes following colonization with S. epi-OVA but not S. epi-control. As shown in FIG. 15A and FIG. 15D, neither the total percentages of CD8+ or CD4+ T cells, respectively, were significantly increased in the draining lymph nodes of mice inoculated with S. epi OVA, as compared to S. epi-control, indicating that the T cell activation was antigen specific, as opposed to a general activation of the immune response. These results indicate that S. epi-OVA elicited an antitumor immune response under conditions of physiologic colonization. Moreover, OVA-expressing bacteria induced activated, antigen-specific CD4+ and CD8+ T cells that migrated to the tumor site.

The localization and antigen requirements for the antitumor effect were further assessed. Mice were colonized with S. epidermidis strains harboring different versions of OVA before injecting B16-OVA tumor cells subcutaneously into the right flank. Since S. epi-wOT1 only expressed the CD8+ T cell antigen, mice were colonized with S. epi-wOVA (i.e., the full-length OVA protein) to determine whether a wall-displayed construct with CD8+ and CD4+ antigens could elicit a response. However, as shown in FIG. 15F, S. epi-wOVA showed no antitumor effect compared to control. In contrast, colonization with a combination of S. epi-wOT1 and S. epi-sOT2 decreased tumor weight (FIG. 15F) and increased IFNγ-expressing CD8+ T cells (data not shown), suggesting that the antitumor efficacy generally needed both a wall-attached CD8+ T cell antigen and a secreted CD4+ T cell antigen. When the localization and antigenic peptide identity were mismatched by colonizing mice with S. epi-wOT2 and S. epi-sOT1, no reduction in tumor weights (FIG. 15F) and no increases in the percentage of IFNγ-expressing CD4+ T cells (FIG. 15G) and CD8+ T cells (FIG. 15H) were observed in tumor-draining lymph nodes. Accordingly, these in vivo data were consistent with a model in which antigen subcellular localization in the bacterial cell is important, where both a wall-attached CD8+ epitope and a secreted CD4+ epitope are generally needed for optimal antitumor activity. These results also suggest that antigen-specific CD4+ and CD8+ T cells are both generally needed for the S. epidermidis-induced antitumor response.

### Example 10—Robust Activation of Anti-Viral Immunity by Targeting Antigen-Presenting Cells with Recombinant S. epidermidis

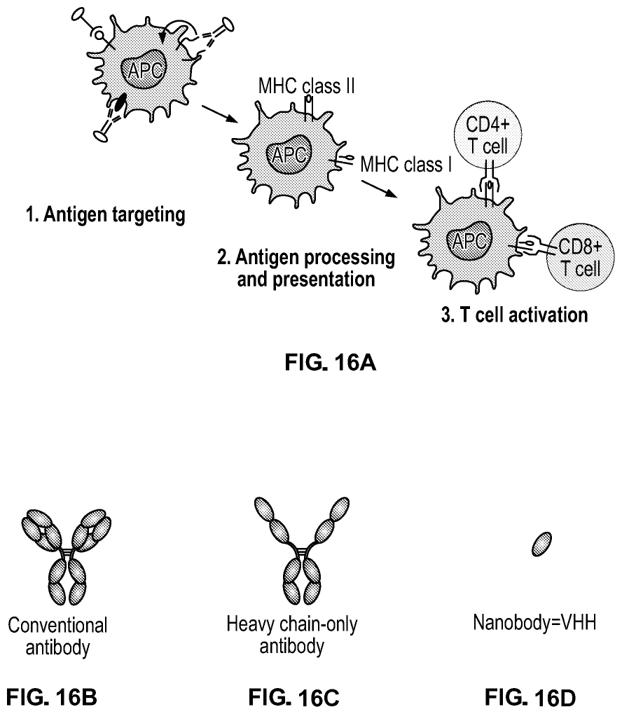

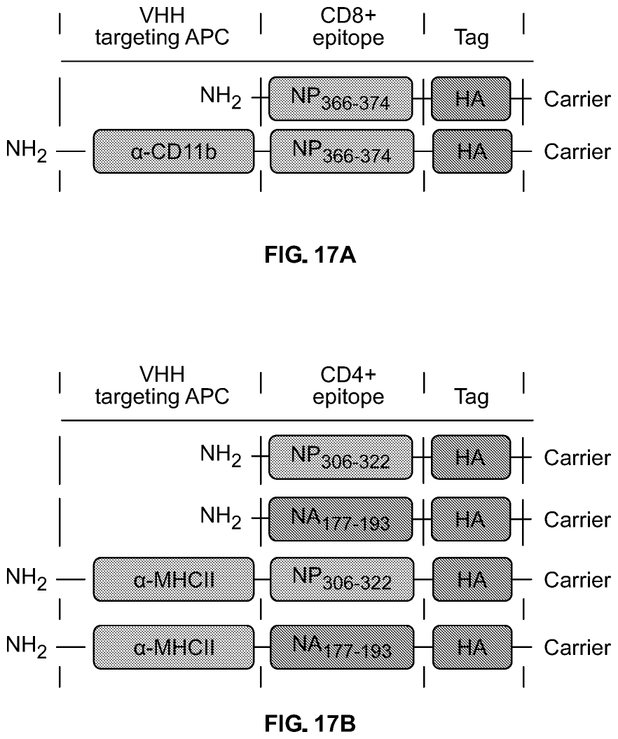

Targeting of antigen-presenting cells (APCs) can be used to promote a robust activation of specific immune cell types. FIG. 16A (adapted from López-Requena, 2012.) illustrates the targeting of APC antigens to promote a specific activation of immune cells. Targeting of CD11b on APCs enhances CD8+ T cell activation, and targeting MHC-II on APCs enhances activation of CD4+ T cells and B cells. FIG. 16B (adapted from López-Requena, 2012.) illustrates functional antibody fragments, including nanobodies (VHH), which can be used in fusion proteins to target specific antigens. FIG. 17A illustrates schematic diagrams of constructs designed to induce a CD8+ T cell-specific response against influenza A virus (IAV) NP₃₆₆₋₃₇₄. Both construct designs include an IAV epitope that promotes a CD8+ T cell response, an HA tag to assess expression, and a carrier to induce localization of the fusion protein either to the cell wall or to induce secretion. The bottom construct also contains a CD11b-targeting VHH fragment, which targets APCs to further promote CD8+ T cell activation. These constructs can be expressed in bacteria such as S. epidermidis and inoculated into subjects to promote an anti-IAV CD8+ T cell response. FIG. 17B shows schematic designs of constructs to induce a CD4+ T cell response. These constructs similarly comprise a carrier and an HA tag, as well as one of two IAV antigen fragments that promote a CD4+ T cell response (NP₃₆₆₋₃₇₄ or NA177.193). Two of the constructs also contain an MHC-II-targeting VHH fragment, which targets APCs to increase CD4+ T cell activation.

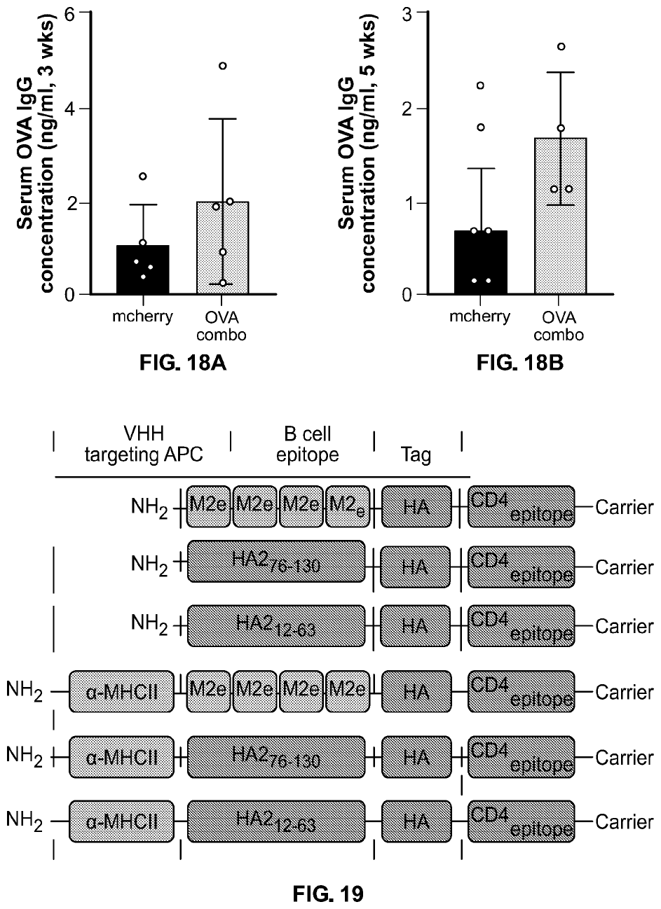

In addition to a T cell response, B cells can also be activated by recombinant bacteria engineered to express heterologous antigen fragments. FIG. 18 shows that mice inoculated with recombinant S. epidermidis expressing ovalbumin constructs have low level induction of ovalbumin-targeting IgG antibodies in the serum at 3 weeks (FIG. 18A) and 5 weeks (FIG. 18B) following inoculation. FIG. 19 shows schematic diagrams of construct designs for expressing heterologous antigens in recombinant bacteria to elicit a B cell response against IAV. All constructs contain a carrier and HA tag, along with B cell-stimulating epitopes ((M2e)₄, HA2₇₆₋₁₃₀, or HA2₁₂₋₆₃). These constructs also contain a CD4+ T cell epitope to promote the activation of B cells by CD4+ T cells. Half of the constructs also contain an MHC-II-targeting VHH fragment, which targets APCs to stimulate B cell and CD4+ T cell activation.

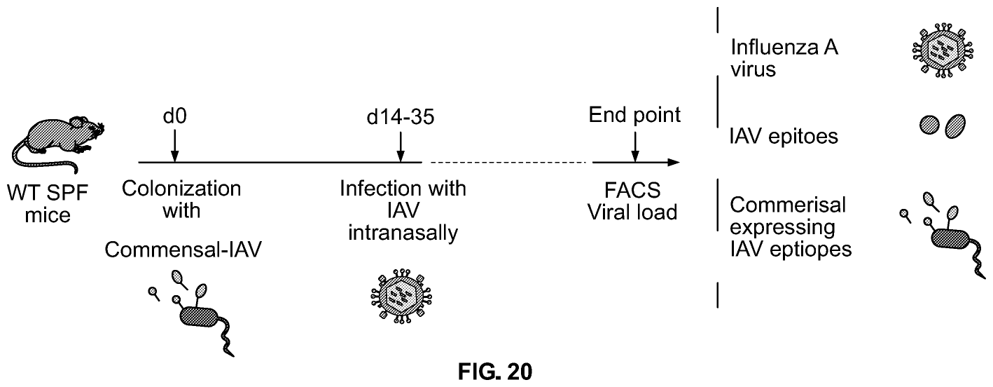

A murine model can be employed to demonstrate the activation of anti-IAV immunity with recombinant bacteria expressing fusion proteins containing IAV antigens and APC-targeting VHH fragments. FIG. 20 illustrates a workflow diagram of an experiment using a murine model to test the effects of recombinant bacteria in promoting an anti-IAV immune response. Wild-type SPF mice can be inoculated with one or more strains of recombinant bacteria, such as S. epidermidis or any other suitable strain, comprising a construct illustrated in FIG. 17A, FIG. 17B, or FIG. 19. After around 14 to 35 days, inoculated mice can be infected with IAV intranasally. At a predetermined endpoint, measures such as survival; weight; body temperature; T cell activation based on Nur77 or IFNγ expression, or any other suitable measure; or B cell activation based on antibody titer, or any other suitable measure, can be used to assess the ability for the recombinant bacteria to induce an anti-IAV immune response.

### Example 11—Engineered S. epidermidis Strains Demonstrate Efficacy in a Metastatic Melanoma Model

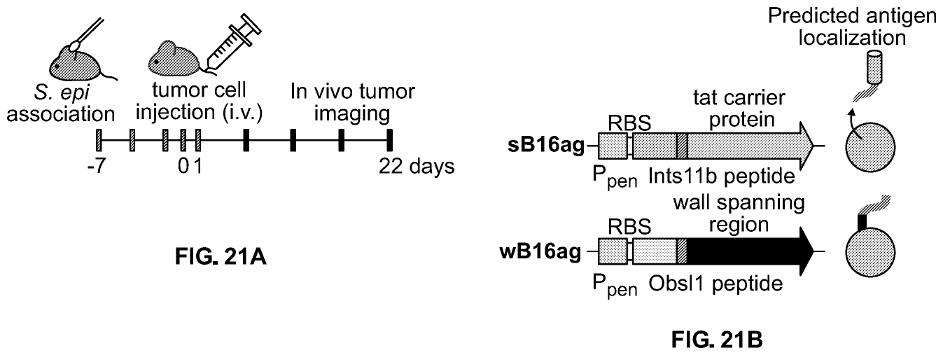

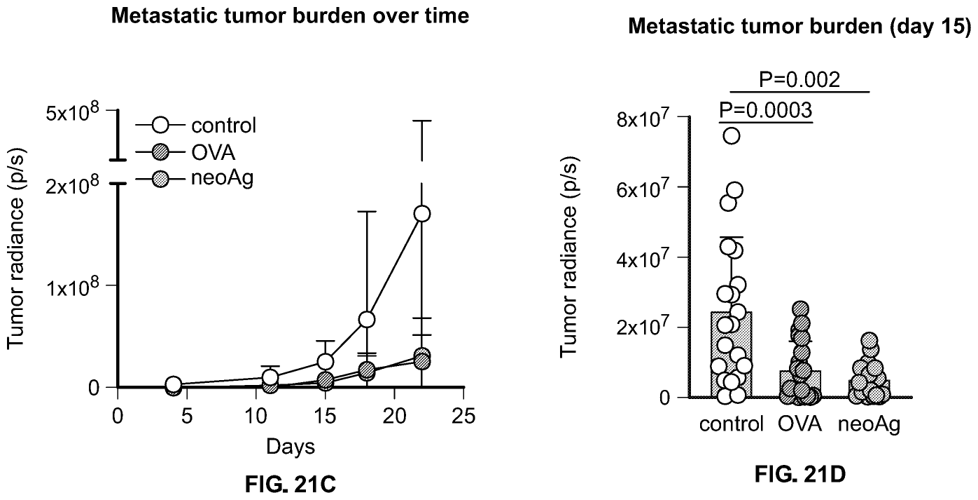

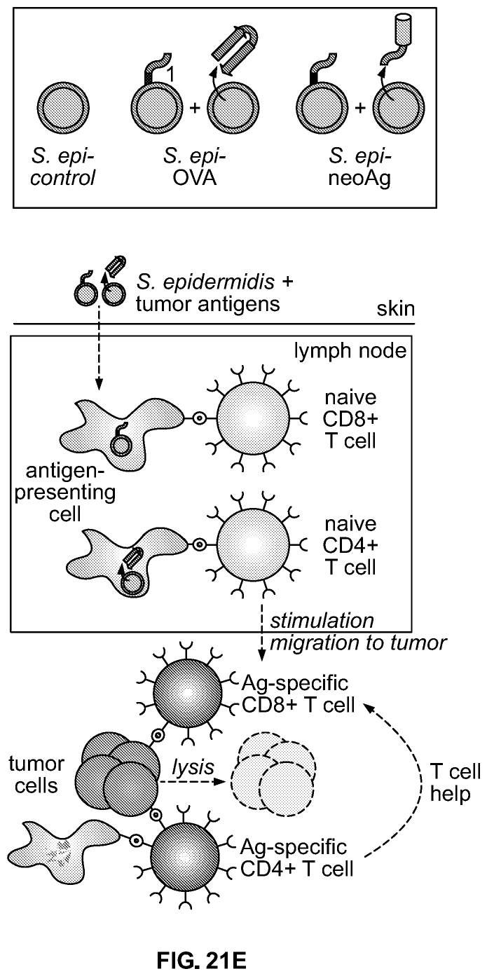

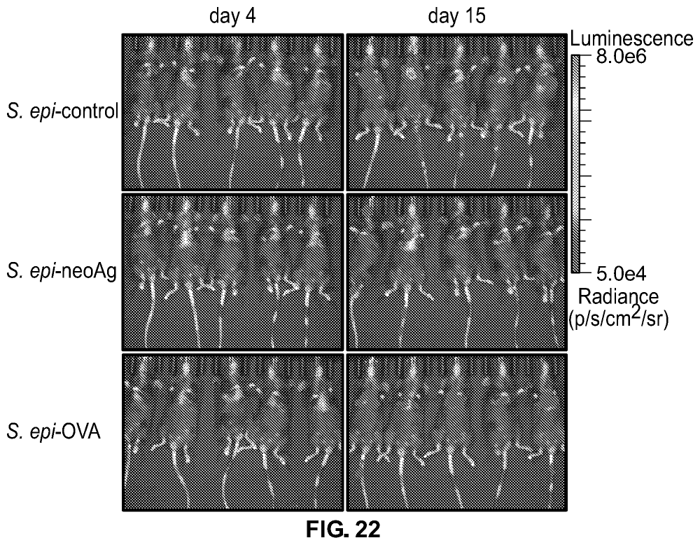

In the above examples, tumor cells were subcutaneously injected into the flank of mice. Although mice were colonized by topical application to the head, murine grooming behavior could distribute S. epidermidis broadly across the skin, raising the question of whether the recombinant bacteria and the tumor need to be in close proximity for the induction of an antitumor immune response. To address this question, experiments were performed in a metastatic melanoma model, whose workflow is schematically illustrated in FIG. 21A, using a cell line derived from B16-F10, a well-characterized (and more aggressive) variant of B16 melanoma. B16-F10-OVA cells constitutively expressing luciferase were injected intravenously, rather than subcutaneously, resulting in metastases in the lungs. Topical association with S. epi-OVA seven days prior to intravenous tumor cell injection substantially slowed tumor progression (FIG. 21C, FIG. 21D, and FIG. 22), demonstrating that the antitumor effect of S. epi-OVA was not restricted to skin and subcutaneous tissues. These data indicated that the antitumor effect of heterologous antigen-expressing S. epidermidis does not generally need an infection or proximity to the tumor, i.e., heterologous antigen-expressing S. epidermidis was capable of stimulating a distal antitumor response relative to the native host niche and successfully targets tumor metastases.

Recombinant bacterial expression of neoantigen-containing peptides naturally present in tumors were next assessed to eliminate the potential issues associated with model antigens in real-world applications, namely their efficient processing in APCs and high expression in syngeneic tumor cell lines. S. epidermidis was engineered to express two neoantigen-containing peptides naturally present in B16-F10 melanoma cells and previously reported to drive an antitumor response when formulated as an mRNA vaccine (S. Kreiter et al., Mutant MHC class II epitopes drive therapeutic immune responses to cancer. Nature. 520, 692-696 (2015).) (FIG. 21). The neoantigen peptide from Obs11(T1764M) preferentially stimulates CD8+ T cells, so a 27-aa peptide centered around the mutated neoantigen residue was spliced into the wall-attachment scaffold described in EXAMPLE 9, yielding strain S. epi-wB16Ag (FIG. 21B, bottom panel). Another neoantigen peptide, Ints11(D314N), primarily stimulates CD4+ T cells, so a 27-aa peptide harboring the neoantigenmutation was spliced into the scaffold for Tat-mediated secretion described in EXAMPLE 9, generating strain S. epi-sB16Ag (FIG. 21B, top panel). Mice were colonized with a mixture of S. epi-wB16Ag and S. epi-sB16Ag (termed “S. epi-neoAg”) and then injected intravenously with B16-F10-OVA-luc cells seven days later. In contrast to S. epi-control, which failed to reduce tumor size, S. epi-neoAg restricted tumor growth at a comparable level to S. epi-OVA (FIG. 21C, FIG. 21D, and FIG. 22). Mice colonized by S. epi-neoAg did not exhibit any symptoms of autoimmunity, consistent with a model in which engineered S. epidermidis-induced T cells are selective for tumor cells over healthy tissue and can be directed against a potentially broad range of host antigens, including neoantigens.

### Example 12—Anchoring Recombinant Proteins to Intractable Organisms Using Sortase A

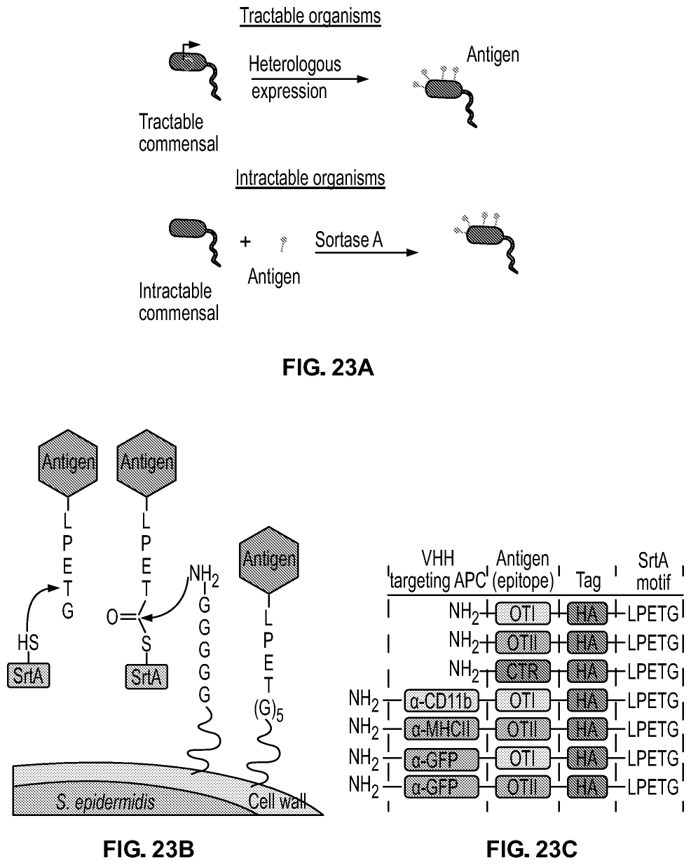

Certain commensal microorganisms, including the gram-positive bacterium Firmicutes can potently modulate the immune response, but have thus far been difficult to study due to the lack of existing genetic engineering tools. To this end, a system using the Staphylococcus aureus transpeptidase Sortase A (SrtA), which is illustrated in FIG. 23A can be employed to anchor fusion proteins to the bacterial cell wall. FIG. 23B illustrates a system, in which a cysteine residue on SrtA reacts with a C-terminal LPXTG motif on the fusion protein. An amine group on the cell wall reacts with the thioester bond linking the fusion protein to SrtA via a nucleophilic acyl substitution. This results in the covalent linkage of the fusion protein to the bacterial cell wall. FIG. 23C shows schematic diagrams of construct designs, which contain an antigen fragment (e.g. OTI, OTII, or CTR), an expression tag (e.g., HA), and a C-terminal LPXTG motif capable of reacting with SrtA. These constructs may also contain an N-terminal VHH region to target APCs (e.g., α-CD11b VHH, α-MHC-II VHH).

### Example 13—Engineered S. epidermidis is Effective Against Established Tumors

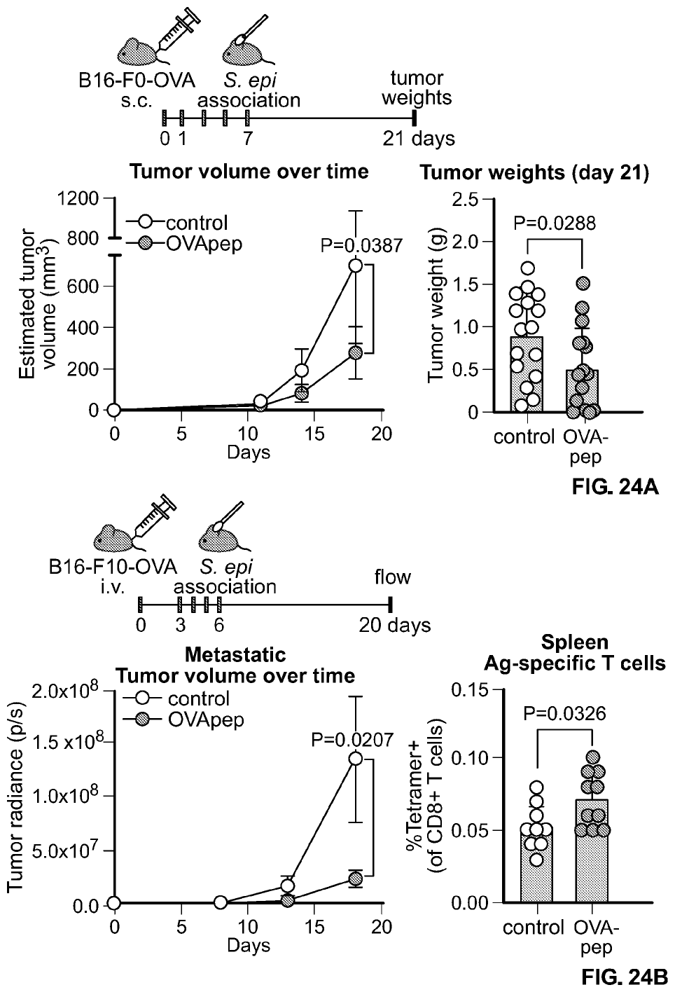

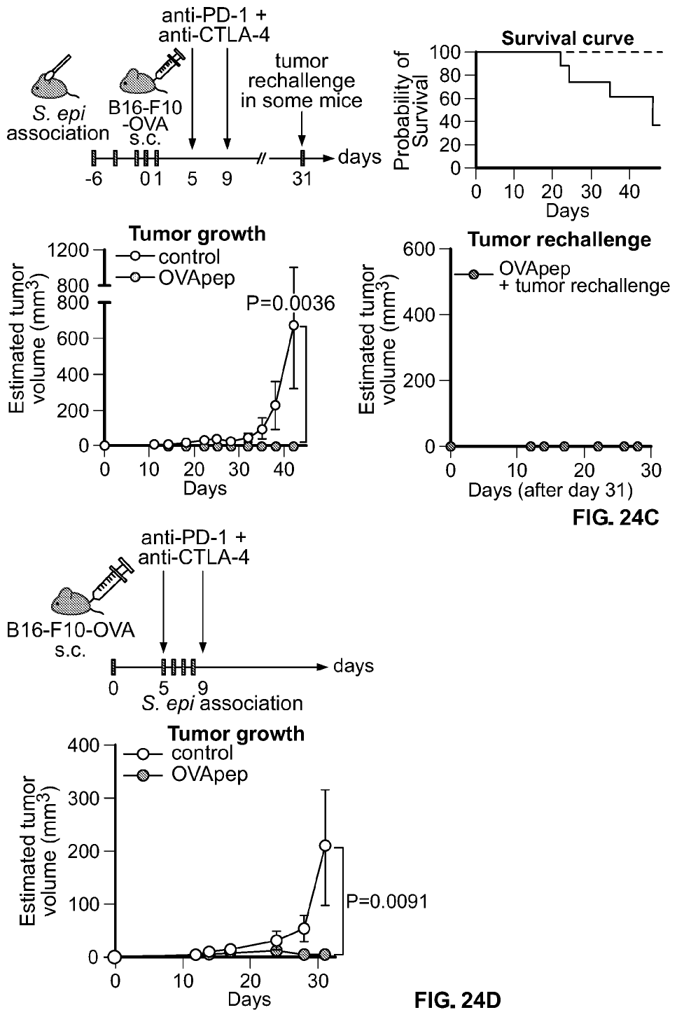

Experiments were performed to test whether colonizing mice with engineered S. epidermidis after tumor cell injection—a model of primary treatment—would yield a therapeutic response. First, mice were injected with B16-F0-OVA cells subcutaneously and then colonized with S. epi-control vs. S. epi-OVApep four times, starting one day after tumor cell injection. A significant reduction in tumor cell burden was observed (FIG. 24A). In a second experiment using B16-F10-OVA in the metastatic melanoma model, with colonization starting three days after intravenous tumor cell injection, the reduction in tumor burden was even more pronounced and was accompanied by an increase in OVA-specific CD8+ T cell induction (FIG. 24B). Given that a measurable increase in S. epidermidis-induced T cells takes at least seven days, the activity observed in ‘treatment mode’ (post-tumor cell injection) demonstrated that engineered S. epidermidis is effective even after an aggressive tumor is established.

During the analysis of S. epidermidis-induced tumor infiltrating lymphocytes (TILs), one observation stood out: the majority of TILs were PD-1+, consistent with the possibility that they were partially or completely exhausted. Reasoning that these cells could exert more potent antitumor activity if co-administered, using the checkpoint inhibitors anti-PD-1 and anti-CTLA-4, mice were colonized in the prophylaxis model and given two doses of an anti-PD-1/anti-CTLA-4 mixture at days 5 and 9 post tumor cell injection. The combination of anti-PD-1, anti-CTLA-4, and S. epi-control was unable to control tumor growth, consistent with the aggressive nature of B16-F10 melanoma. However, when colonized with S. epi-OVApep, a striking response was observed: 15/16 bilateral tumors (in 7/8 mice) were rejected, leading to a large survival benefit (FIG. 24C). In another experiment, tumor cells were injected into the right flank. 14/16 mice were complete responders; after 31 days, the 14 mice were rechallenged by injecting B16-F10 cells in the left flank. 14/14 showed no evidence of tumor growth in the left flank, and 9/14 maintained undetectable tumors in the right flank. This data shows that checkpoint blockade enhances the antitumor effect of engineered S. epidermidis, and that this approach yields immunologic memory against the tumor.

A combination of engineered S. epidermidis and checkpoint inhibition was tested to determine if such a combination could yield an enhanced response in the model of primary treatment. Five days after subcutaneous injection of B16-F10-OVA, mice were colonized with S. epi-control or S. epi-OVApep and simultaneously administered a combination of anti-PD1 and anti-CTLA-4 (FIG. 24D). The reduction in tumor burden was pronounced, with rejection of 12/14 tumors (in 5/7 mice with bilateral tumors). These data show that combining a tumor-expressing commensal with checkpoint blockade could be a viable therapeutic strategy.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A live, recombinant commensal bacterium engineered to express a fusion protein, the fusion protein comprising:
(a) a non-native protein or peptide, and
(b) (i) a tat signal sequence peptide, a sec signal sequence peptide, or a sortase-derived signal sequence peptide, and/or an antigen-presenting cell (APC) targeting moiety, or (ii) a tat signal sequence peptide, a sec signal sequence peptide, or a sortase-derived signal sequence peptide, wherein administration of the bacterium to the host results in colonization of a native host niche by the bacterium, and generation of an adaptive immune response by the host against the non-native protein or peptide.

2. The live, recombinant commensal bacterium of claim 1, wherein the non-native protein or peptide is associated with a host disease or condition selected from the group consisting of:
(i) a cancer;
(ii) an autoimmune disorder; and
(iii) an infection that occurs at or is otherwise associated with a mucosal boundary of the host.

3. The live, recombinant commensal bacterium of claim 1 or 2, wherein the signal sequence peptide:
(i) directs tethering of the expressed fusion protein to a cell wall of the bacterium; or
(ii) directs secretion of the fusion protein from the bacterium following expression.

4. The live, recombinant commensal bacterium of any one of claims 1-3, wherein the tat signal sequence peptide comprises a sequence derived from fepB of Staphylococcus aureus, the sec signal sequence peptide comprises a sequence derived from predicted sec-secreted Staphylococcus epidermidis protein (gene locus HMPREF9993_06668), or the sortase-derived signal sequence peptide comprises one or more sequences derived from Protein A of S. aureus.

5. The live, recombinant commensal bacterium of claim 3 or 4, wherein the signal sequence peptide is fused to the N-terminal side of the non-native protein or peptide and the fusion protein comprises a cell-wall spanning peptide domain on the C-terminal side of the non-native protein or peptide.

6. The live, recombinant commensal bacterium of any one of claims 1-5, wherein the APC targeting moiety comprises a CD11b or MHCII targeting moiety.

7. The live, recombinant commensal bacterium of any one of claims 1-6, wherein the native host niche is selected from the group consisting of the gastrointestinal tract, respiratory tract, urogenital tract, and skin.

8. The live, recombinant commensal bacterium of any one of claims 1-7, wherein the adaptive immune response is distal from the site of administration and/or the native host niche.

9. The live, recombinant commensal bacterium of claim 8, wherein the distal adaptive immune response comprises an immune response in an organ that is not the organ of the site of administration and/or the native host niche, and optionally wherein the site of administration and/or the native host niche comprises skin.

10. The live, recombinant commensal bacterium of claim 8, wherein the distal adaptive immune response comprises an antitumor response, optionally wherein the antitumor response targets a metastasis.

11. The live, recombinant commensal bacterium of any one of claims 1-10, wherein the colonization of the native host niche is persistent or transient.

12. The live, recombinant commensal bacterium of claim 11, wherein the native host niche is persistently colonized, and wherein colonization is for at least 60 days, at least 112 days, at least 178 days, at least 180 days, at least 1 year, at least 2 years, or at least 5 years.

13. The live, recombinant commensal bacterium of claim 11 or 12, wherein the persistent colonization provides a persistent antigen source, optionally wherein the antigen stimulates an antigen-specific T cell population and produces a persistent antigen-specific T cell population.

14. The live, recombinant commensal bacterium of claim 11, wherein the native host niche is transiently colonized, and wherein colonization is for 1 day to 60 days, 3.5 days to 60 days, or 7 days to 28 days.

15. The live, recombinant commensal bacterium of any one of claims 1-14, wherein the fusion protein comprises the non-native protein or peptide fused to the N-terminus or the C-terminus of a native bacterial protein or portion thereof.

16. The live, recombinant commensal bacterium of any one of claims 1-15, wherein the bacterium is formulated for administration in combination with a high-complexity defined microbial community.

17. The live, recombinant commensal bacterium of any one of claims 1-16, wherein the live, recombinant commensal bacterium is
(i) a Gram-positive bacterium selected from the group consisting of Staphylococcus epidermidis, Faecalibacterium sp., Corynebacterium spp., Eubacterium limosum, Ruminococcaceae bacterium cv2, Clostridium sp., Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Ruminococcus bromii L2-63, Firmicutes bacterium ASF500, Firmicutes bacterium ASF500, Bifidobacterium animalis subsp. lactis ATCC 27673, Bifidobacterium breve, Cutibacterium acnes, Cutibacterium avidum, Dolosigranulum pigrum, Finegoldia magna, Rothia mucilaginosa, Streptococcus pyogenes, Streptococcus agalactiae, Streptococcus gordonii, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium longum, Gardnerella vaginalis, Atopobium vaginae, Mobiluncus mulieris, Mageeibacillus indolicus, Enterococcus faecium, and Lactococcus lactis, and optionally wherein the bacterium is S. epidermidis NIHLM087; or
(ii) a Gram-negative bacterium selected from the group consisting of Bacteroides thetaiotaomicron, Helicobacter hepaticus, Parabacteroides sp., Moraxella catarrhalis, Moraxella nonliquefaciens, Haemophilus influenzae, Haemophilus aegyptius, Neisseria lactamica, Neisseria cinerea, Neisseria mucosa, Veillonella parvula, Prevotella bivia, Prevotella buccalis, Gardnerella vaginalis, and Mobiluncus mulieris.

18. A method of treating a disease or condition in a subject, comprising: administering a live, recombinant commensal bacterium engineered to express a heterologous antigen to a subject, wherein the expressed heterologous antigen induces an antigen-specific immune response to treat the disease or condition in the subject.

19. A method of treating a disease or condition in a subject, comprising: administering the live, recombinant commensal bacterium of any one of claims 1-17 to a subject, wherein the adaptive immune response to the non-native protein or peptide treats the disease or condition in the subject.

20. The method of claim 18 or 19, wherein the administration is via a route selected from the group consisting of topical, enteral, parenteral and inhalation.

21. The method of any one of claims 18-20, wherein the method further comprises co-administering one or more additional agents, and optionally wherein the one or more additional agents comprises one or more checkpoint inhibitors.

22. A pharmaceutical composition comprising a live, recombinant commensal bacterium of any of claims 1-17.

23. A live, recombinant commensal bacterium, wherein the bacterium is engineered to express (a) a first non-native protein or peptide, wherein the first non-native protein or peptide is engineered to elicit a CD4+ T cell response, and
(b) a second non-native protein or peptide, wherein the second non-native protein or peptide is engineered to elicit a CD8+ cytotoxic T cell response, and
wherein administration of the bacterium to a host results in colonization of a native host niche by the bacterium.

24. A composition comprising:
(a) a first live, recombinant commensal bacterium engineered to express a first non-native protein or peptide, wherein the first non-native protein or peptide is engineered to elicit a CD4+ T cell response, and
(b) a second live, recombinant commensal bacterium engineered to express a second non-native protein or peptide, wherein the second non-native protein or peptide is engineered to elicit a CD8+ cytotoxic T cell response, and
wherein administration of the composition to a host results in colonization of a native host niche by the first live, recombinant commensal bacterium and the second live, recombinant commensal bacterium.

25. The live, recombinant commensal bacterium of claim 23 or composition of claim 24, wherein the first non-native protein or peptide and the second non-native protein or peptide are each derived from a shared antigen or a different antigen, and optionally when the first non-native protein or peptide and the second non-native protein or peptide are derived from the shared antigen, the first non-native protein or peptide and the second non-native protein or peptide comprise different amino acid sequences.

26. The live, recombinant commensal bacterium of claim 23 or 25, or the composition of claim 24 or 25, wherein the first non-native protein or peptide comprises a signal sequence peptide that directs secretion of the non-native protein or peptide from the first live, recombinant commensal bacterium following expression, and/or the second non-native protein or peptide comprises a second signal sequence peptide that directs covalent attachment of the second non-native protein or peptide to a cell wall of the second live, recombinant commensal bacterium following expression.

27. A method of treating a disease or condition in a host, comprising: administering the live, recombinant commensal bacterium of any one of claims 23, 25, or 26, or the composition of any one of claims 24-26 to the host, wherein the elicited CD4+ T cell response and CD8+ cytotoxic T cell response treats the disease or condition in the host.

28. A bacterial surface display system comprising: (a) a fusion protein comprising a cell-surface tethering moiety and a non-native protein or peptide; (b) a bacterium; and (c) a protein or gene encoding the same capable of catalyzing a covalent attachment of the cell-surface tethering moiety to a cell wall protein or outer membrane protein of the bacterium thereby displaying the fusion protein on a bacterial surface.

29. A bacterial surface display system comprising: (a) a fusion protein comprising a cell-surface tethering moiety and a non-native protein or peptide and (b) a bacterium, wherein the fusion protein is covalently attached to a cell wall protein or outer membrane protein via the cell-surface tethering moiety, and wherein the covalent attachment was catalyzed by a protein capable of catalyzing attachment of the cell-surface tethering moiety to the cell wall protein or outer membrane protein of the bacterium.

30. The bacterial surface display system of claim 28 or 29 wherein the cell-surface tethering moiety comprises a Sortase A (SrtA) motif and the protein capable of catalyzing the covalent attachment is a SrtA protein.

31. The bacterial surface display system of claim 30, wherein the SrtA motif and/or the SrtA protein is derived from S. aureus, optionally wherein the SrtA motif comprises the amino acid sequence LPXTG.

32. The bacterial surface display system of any one of claims 28-31, wherein the fusion protein comprises an antigenic protein or peptide associated with a host disease or condition selected from the group consisting of a proliferative disorder, an autoimmune disorder, and an infection.

33. The bacterial surface display system of any one of claims 28-32, wherein administration of the bacterium to a host results in colonization of a native host niche by the bacterium eliciting a T-cell response to the non-native protein or peptide.

34. The bacterial surface display system of any one of claims 28-33, wherein the fusion protein further comprises an antigen-presenting cell (APC) targeting moiety, optionally wherein the APC targeting moiety comprises a CD11b or a MHC II targeting moiety.

35. The bacterial surface display system of claim of any one of claims 28-34, wherein the bacterium is
(i) a Gram-positive bacterium selected from the group consisting of Staphylococcus epidermidis, Faecalibacterium sp., Corynebacterium spp., Eubacterium limosum, Ruminococcaceae bacterium cv2, Clostridium sp., Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Ruminococcus bromii L2-63, Firmicutes bacterium ASF500, Firmicutes bacterium ASF500, Bifidobacterium animalis subsp. lactis ATCC 27673, Bifidobacterium breve, Cutibacterium acnes, Cutibacterium avidum, Dolosigranulum pigrum, Finegoldia magna, Rothia mucilaginosa, Streptococcus pyogenes, Streptococcus agalactiae, Streptococcus gordonii, Lactobacillus crispatus, Lactobacillus jensenii Gasser, Lactobacillus gasseri, Lactobacillus iners, Lactobacillus acidophilus, Lactobacillus johnsonii, Lactobacillus rhamnosus, Lactobacillus casei, Lactobacillus helveticus, Lactobacillus reuteri, Lactobacillus salivarius, Bifidobacterium longum, Gardnerella vaginalis, Atopobium vaginae, Mobiluncus mulieris, Mageeibacillus indolicus, Enterococcus faecium, and Lactococcus lactis, and optionally wherein the bacterium is S. epidermidis NIHLM087; or
(ii) a Gram-negative bacterium selected from the group consisting of Bacteroides thetaiotaomicron, Helicobacter hepaticus, Parabacteroides sp., Moraxella catarrhalis, Moraxella nonliquefaciens, Haemophilus influenzae, Haemophilus aegyptius, Neisseria lactamica, Neisseria cinerea, Neisseria mucosa, Veillonella parvula, Prevotella bivia, Prevotella buccalis, Gardnerella vaginalis, and Mobiluncus mulieris.

36. A pharmaceutical composition comprising the bacterial surface display system of any one of claims 28-35, and an excipient.

37. The pharmaceutical composition of claim 36, wherein the pharmaceutical composition further comprises a high-complexity defined microbial community.

38. A method of treating a disease or condition in a host, comprising: administering the bacterial surface display system of claim 28-35, or pharmaceutical composition of claim 36 or 37, to the host, wherein the administration results in colonization of a native host niche in the host by the bacterium, internalization of the bacterium or the non-native protein or peptide by an antigen-presenting cell, presentation of an antigen derived from the non-native protein or peptide by the antigen-presenting cell within an MHC-I or MHC-II complex, and generation of a T-cell response to the antigen, and wherein the T-cell response treats the disease or condition in the host.

39. The method of claim 38, wherein the colonization of the native host niche is persistent or transient.

40. The method of claim 39, wherein the native host niche is transiently colonized, and wherein colonization is for 1 day to 60 days, 3.5 days to 60 days, or 7 days to 28 days.

41. The method of claim 39, wherein the native host niche is selected from the group consisting of the gastrointestinal tract, respiratory tract, urogenital tract, and skin.

42. A live, recombinant commensal bacterium of any one of claims 1-17, composition of any one of claims 22, 24-26, or method of any one of claim 27, or 38-41, wherein the host is a subject.

43. A live, recombinant commensal bacterium or composition of 42, or method of any one of claims 18-21, or 38-42, wherein the subject is a human.


SEQUENCE LISTING (61 sequences)

SEQ ID NO: 1 - protein, 8 aa, Artificial Sequence
  Synthetic Peptide
SIINFEKL

SEQ ID NO: 2 - protein, 17 aa, Artificial Sequence
  Synthetic Peptide
ISQAVHAAHAEINEAGR

SEQ ID NO: 3 - protein, 55 aa, Artificial Sequence
  Synthetic Peptide
MVLVNAIVFKGLWPFASGTMSMLVLLPDEVSGLEQLESIINFEKLTEWSTQHKVQ

SEQ ID NO: 4 - protein, 21 aa, Artificial Sequence
  Synthetic Peptide
MEVGWYRSPFSRVVHLYRNGK

SEQ ID NO: 5 - protein, 15 aa, Artificial Sequence
  Synthetic Peptide
SHLVEALYLVCGEEG

SEQ ID NO: 6 - protein, 10 aa, Artificial Sequence
  Synthetic Peptide
SRLGLWVRME

SEQ ID NO: 7 - protein, 11 aa, Artificial Sequence
  Synthetic Peptide
LQTLALWSRMD

SEQ ID NO: 8 - protein, 24 aa, Artificial Sequence
  Sythentic Peptide
ECCARCLVGAPFASLVATGLCFFG

SEQ ID NO: 9 - protein, 21 aa, Artificial Sequence
  Synthetic Peptide
LLLAEGFYTTGAVRQIFGDYK

SEQ ID NO: 10 - protein, 32 aa, Artificial Sequence
  Synthetic Peptide
VYIYFNTWTTCQSIAFPSKTSASIGSLCADAR

SEQ ID NO: 11 - protein, 25 aa, Artificial Sequence
  Synthetic Peptide
QMTFHLFIAAFVGAAATLVSLLTFM

SEQ ID NO: 12 - protein, 21 aa, Artificial Sequence
  Synthetic Peptide
RPSQRSKYLATASTMDHARHG

SEQ ID NO: 13 - protein, 21 aa, Artificial Sequence
  Synthetic Peptide
KQHYLLYIWQGSQASQDEIAA

SEQ ID NO: 14 - protein, 27 aa, Artificial Sequence
  Synthetic Peptide
MSPKVDVFTANTSLSSGPLPTFPLEQL

SEQ ID NO: 15 - protein, 25 aa, Artificial Sequence
  Synthetic Peptide
STEDFTRALGMTPAAFSALPRWKQQ

SEQ ID NO: 16 - protein, 20 aa, Artificial Sequence
  Synthetic Peptide
VKGESLFHSSKSMDLRVNGE

SEQ ID NO: 17 - protein, 9 aa, Artificial Sequence
  Synthetic Peptide
ASNENMETM

SEQ ID NO: 18 - protein, 17 aa, Artificial Sequence
  Synthetic Peptide
LLQNSQVYSLIRPNENP

SEQ ID NO: 19 - protein, 17 aa, Artificial Sequence
  Synthetic Peptide
VAWSASACHDGMGWLTI

SEQ ID NO: 20 - protein, 24 aa, Artificial Sequence
  Synthetic Peptide
MSLLTEVETPIRNEWGCRCNDSSD

SEQ ID NO: 21 - protein, 52 aa, Artificial Sequence
  Synthetic Peptide
GGWTGMIDGWYGYHHQNEQGSGYAADQKSTQNAINGITNKVNTVIEKMNIQF

SEQ ID NO: 22 - protein, 55 aa, Artificial Sequence
  Synthetic Peptide
RIQDLEKYVEDTKIDLWSYNAELLVALENQHTIDLTDSEMNKLFEKTRRQLRENA

SEQ ID NO: 23 - protein, 904 aa, Human herpesvirus 1 (strain KOS) (HHV-1) (Human herpes simplex virus 1)
MHQGAPSWGRRWFVVWALLGLTLGVLVASAAPTSPGTPGVAAATQAANGGPATPAPPPLGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYQANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 24 - protein, 394 aa, Human herpesvirus 1 (strain 17) (HHV-1) (Human herpes simplex virus 1)
MGGAAARLGAVILFVVIVGLHGVRSKYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRHTQKAPKRIRLPHIREDDQPSSHQPLFY

SEQ ID NO: 25 - protein, 8 aa, Artificial Sequence
  Synthetic Peptide
SSIEFARL

SEQ ID NO: 26 - protein, 1273 aa, Severe acute respiratory syndrome coronavirus 2 (2019-nCoV) (SARS-CoV-2)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - protein, 455 aa, Human immunodeficiency virus 1
VPVWRDADTTLFCASDAKSHVTEAHNVWATHACVPTDPNPQEIHLENVTENFNMWKNNMVEQMQEDVISLWEQSLKPCVKLTPLCVTLNCTNANLTNANLTNANNITNVENITDEVRNCSFNVTTDLRDKQQKVHALFYRLDIVQINSKNSSDYRLINCNTSVIKQACPKISFDPIPIHYCTPAGYAILKCNDKNFNGTGPCKNVSSVQCTHGIKPVVSTQLLLNGSLAEEEIIIRSENLTNNVKTIIVHLNKSVEINCTRPSNNTRTSITIGPGQVFYRTGDIIGDIRKVSCELNGTKWNEVLKQVKEKLKEHFNKNISFQPPSGGDLEITMHHFSCRGEFFYCNTTQLFNNTYSNGTITLPCKIKQIINMWQGVGQAMYAPPISGRINCLSNITGLLLTRDGNNGTNETFRPGGGNIKDNWRSELYKCKVVQIEPLGIAPTRAKRRVVEREKK

SEQ ID NO: 28 - protein, 143 aa, Human immunodeficiency virus 1
MGAASLTLTVQARQLLSGIVQQQNDLLRAIEAQQHLLQLTVWGIKQLQARVLAVERYLKDQQLLGIWGCSGKLICTTAVPWNSSWSNKSQDQIWHNMTWMEWEREIENYTDLIYTLIEKSQNQQEKNEQELLELDKWASLWNW

SEQ ID NO: 29 - protein, 72 aa, Human immunodeficiency virus 1
CTDYSGNATNANSTNTTNNSTGTLGNIEMKNCSFNITTSIREKKKEYALFYRVDVVPIDNDINNTRYRLISC

SEQ ID NO: 30 - protein, 35 aa, Artificial Sequence
  Synthetic Peptide
CTRPNNNTRKSIRIQRGPGRAFVTIGKIGNMRQAH

SEQ ID NO: 31 - protein, 11 aa, Artificial Sequence
  Synthetic Peptide
SSGGDPEIVTH

SEQ ID NO: 32 - protein, 22 aa, Artificial Sequence
  Synthetic Peptide
ELDKWASLWNWFNITNWLWYIK

SEQ ID NO: 33 - protein, 131 aa, Artificial Sequence
  Synthetic Peptide
QVQLQESGGGLVQAGDSLRLSCAASGRTFSRGVMGWFRRAPGKEREFVAIFSGSSWSGRSTYYSDSVKGRFTISRDNAKNTVYLQMNGLKPEDTAVYYCAAGYPEAYSAYGRESTYDYWGQGTQVTVSSGG

SEQ ID NO: 34 - protein, 120 aa, Artificial Sequence
  Synthetic Peptide
QVQLQESGGGLVQAGGSHNLSCTASGITFSSLAMGWFRQTPGKEREFVANIMRSGSSVFYADSVRGRFTISRDNAKNTAHLQMNSLKPEDTAVYFCAATRGAWPAEYWGQGTQVTVSSGG

SEQ ID NO: 35 - DNA, 24 bp, Artificial Sequence
  Synthetic Polynucleotide
agtataatcaactttgaaaaactg

SEQ ID NO: 36 - DNA, 51 bp, Artificial Sequence
  Synthetic Polynucleotide
atttcccaggctgttcatgccgcacatgctgagatcaatgaggcaggacgt

SEQ ID NO: 37 - DNA, 167 bp, Artificial Sequence
  Synthetic Polynucleotide
atggtgttggtgaatgcgattgtctttaaaggattatggccttttgcatcaggtacaatgtcaatgttggtcttattgccagacgaagtttctggcttagaacagttagaatcaatcataaactttgaaaaattaactgagtggtctacacaacataaggttcaatc

SEQ ID NO: 38 - DNA, 63 bp, Artificial Sequence
  Synthetic Polynucleotide
atggaagtcggttggtatcgttcccctttttcacgtgtggtgcatctttaccgcaacgggaaa

SEQ ID NO: 39 - DNA, 45 bp, Artificial Sequence
  Synthetic Polynucleotide
agccacttagtcgaagccctttacctggtttgcggggaagagggt

SEQ ID NO: 40 - DNA, 30 bp, Artificial Sequence
  Synthetic Polynucleotide
tctcgcttgggattatgggttcgtatggaa

SEQ ID NO: 41 - DNA, 33 bp, Artificial Sequence
  Synthetic Polynucleotide
ttgcaaaccttggcgctgtggtcgcgcatggat

SEQ ID NO: 42 - DNA, 72 bp, Artificial Sequence
  Synthetic Polynucleotide
gagtgttgcgcccgttgcttagttggcgctcctttcgcgtcattggtagccacgggtttgtgcttctttggc

SEQ ID NO: 43 - DNA, 63 bp, Artificial Sequence
  Synthetic Polynucleotide
ttacttttagcggagggcttttacacaacgggtgccgttcgtcagatcttcggtgactataaa

SEQ ID NO: 44 - DNA, 96 bp, Artificial Sequence
  Synthetic Polynucleotide
gtatacatctacttcaacacgtggacgacgtgtcaatcgatcgcctttccgtcgaagacttcagcctctattggaagcctgtgcgctgacgcccgc

SEQ ID NO: 45 - DNA, 75 bp, Artificial Sequence
  Synthetic Polynucleotide
cagatgacctttcacctgttcatcgcggcttttgtcggagcggctgccaccttagtcagtttattaacatttatg

SEQ ID NO: 46 - DNA, 63 bp, Artificial Sequence
  Synthetic Polynucleotide
cgccccagccagcgctcgaaatatctggccacagcctcaacaatggatcatgctcgccacgga

SEQ ID NO: 47 - DNA, 63 bp, Artificial Sequence
  Synthetic Polynucleotide
aaacagcactacctgttatacatctggcaaggctcccaggcttctcaagatgaaattgctgct

SEQ ID NO: 48 - DNA, 81 bp, Artificial Sequence
  Synthetic Polynucleotide
atgagtcccaaagttgatgtttttactgcaaatacctcccttagttcgggacctttaccaacttttcccttggaacagttg

SEQ ID NO: 49 - DNA, 75 bp, Artificial Sequence
  Synthetic Polynucleotide
tccactgaggatttcacacgcgcccttggtatgaccccagcagccttctctgctttgccacgttggaagcaacag

SEQ ID NO: 50 - DNA, 60 bp, Artificial Sequence
  Synthetic Polynucleotide
gtaaaaggcgaatcccttttccacagctctaagtcgatggatcttcgtgtgaatggagaa

SEQ ID NO: 51 - DNA, 27 bp, Artificial Sequence
  Synthetic Polynucleotide
gccagcaacgagaacatggagaccatg

SEQ ID NO: 52 - DNA, 51 bp, Artificial Sequence
  Synthetic Polynucleotide
ctgctgcagaacagccaggtgtacagcctgatcaggcccaacgagaacccc

SEQ ID NO: 53 - DNA, 51 bp, Artificial Sequence
  Synthetic Polynucleotide
gtggcctggagcgccagcgcctgccacgacggcatgggctggctgaccatc

SEQ ID NO: 54 - DNA, 72 bp, Artificial Sequence
  Synthetic Polynucleotide
atgagcctgctgaccgaggtggagacccccatcaggaacgagtggggctgcaggtgcaacgacagcagcgac

SEQ ID NO: 55 - DNA, 156 bp, Artificial Sequence
  Synthetic Polynucleotide
ggcggctggaccggcatgatcgacggctggtacggctaccaccaccagaacgagcagggcagcggctacgccgccgaccagaagagcacccagaacgccatcaacggcatcaccaacaaggtgaacaccgtgatcgagaagatgaacatccagttc

SEQ ID NO: 56 - DNA, 165 bp, Artificial Sequence
  Synthetic Polynucleotide
aggatccaggacctggagaagtacgtggaggacaccaagatcgacctgtggagctacaacgccgagctgctggtggccctggagaaccagcacaccatcgacctgaccgacagcgagatgaacaagctgttcgagaagaccaggaggcagctgagggagaacgcc

SEQ ID NO: 57 - DNA, 2715 bp, Human alphaherpesvirus 1 (Herpes simplex virus type 1)
atgcaccagggcgccccctcgtgggggcgccggtggttcgtcgtatgggcgctcttggggttgacgctgggggtcctggtggcgtcggcggctccgacttcccccggcacgcctggggtcgcggccgcgacccaggcggcgaacgggggccctgccactccggcgccgccgccccttggcgccgccccaacgggggacccgaaaccgaagaagaacaaaaaaccgaaaaacccaacgccaccacgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgctggcgactggcgactttgtgtacatgtccccgttttacggctaccgggaggggtcgcacaccgaacacaccacgtacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaagtggacgagatgctgcgctccgagtacggcggctccttccgattctcctccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactaccaggccaatgggggctttctgatcgcgtaccagccccttctcagcaacacgctcgcggagctgtacgtgcgggaacacctccgagagcagagccgcaagcccccaaaccccacgcccccgccgcccggggccagcgccaacgcgtccgtggagcgcatcaagaccacctcctccatcgagttcgcccggctgcagtttacgtacaaccacatacagcgccatgtcaacgatatgttgggccgcgttgccatcgcgtggtgcgagctacagaatcacgagctgaccctgtggaacgaggcccgcaagctgaaccccaacgccatcgcctcggtcaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgttcgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgttacgtcatgcggctgcagagcaaccccatgaaggccctgtaccctctaaccaccaaggagctcaagaaccccaccaacccggacgcgtccggggagggcgaggagggcggcgactttgacgaggccaagctagccgaggccagggagatgatacggtacatggccctggtgtcggccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 58 - DNA, 1185 bp, Human alphaherpesvirus 1 (Herpes simplex virus type 1)
atggggggggctgccgccaggttgggggccgtgattttgtttgtcgtcatagtgggcctccatggggtccgcagcaaatatgccttggtggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccacactcaaaaagccccaaagcgcatacgcctcccccacatccgggaagacgaccagccgtcctcgcaccagcccttgttttactag

SEQ ID NO: 59 - DNA, 24 bp, Artificial Sequence
  Synthetic Polynucletodie
agcagcatcgagttcgccaggctg

SEQ ID NO: 60 - DNA, 3822 bp, Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 61 - protein, 119 aa, Artificial Sequence
  Synthetic Peptide
MAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGG